(19) United States
(12) Patent Application Publication
(10) Pub. No.: US 2024/0196909 A1
(43) Pub. Date: Jun. 20, 2024

(54) Bio-control methods for Xylella and Xanthomonas Bacteria

(71) Applicants: Ting-Yu Yeh, Baltimore, MD (US); Gregory Contreras, Baltimore, MD (US)

(72) Inventors: Ting-Yu Yeh, Baltimore, MD (US); Gregory Contreras, Baltimore, MD (US)

(21) Appl. No.: 18/068,594

(22) Filed: Dec. 20, 2022

(51) Int. Cl. A01N 63/40; A01P 1/00; C12N 15/74

(52) U.S. Cl. CPC A01N 63/40; A01P 1/00; C12N 15/74; C12N 2795/00022; C12N 2795/00032

(57) ABSTRACT

The present invention describes applications and methods to use (1) recombinant Xanthomonas phages (phiXfu, Xfv, Xfr, fXa, phiXca), and their variants to prevent and eradicate the Xanthomonas pathogens causing black rot, bacterial blight, bacterial leaf spot, bacterial blight of bean, sugarcane leaf scald, cassava bacterial blight, etc. (2) recombinant Xylella phages (fXy1, fXy2, fXy3, fXy4, fXy5, fXy6, fXy7, fXy8, fXy9, and fXy10) and their variants to prevent and eradicate Xylella fastidiosa, which causes olive quick decline syndrome (OQDS), Pierce's disease (grape), phoney peach disease, bacterial leaf scorch, oleander leaf scorch, citrus variegated chlorosis disease, and so on.

### FIELD

The disclosure provides novel recombinant bacteriophage nucleic acid sequences that can eradicate plant bacterial Xylella and Xanthomonas pathogens causing variety of plant diseases, such as olive quick decline syndrome (OQDS), Pierce's disease (grape), phoney peach disease, bacterial leaf scorch, oleander leaf scorch, citrus variegated chlorosis disease, black rot, bacterial blight, bacterial leaf spot, bacterial blight of bean, sugarcane leaf scald, cassava bacterial blight, etc.

### BACKGROUND

The family Xanthomonadaceae is a wide-spread family of bacteria belonging to the gamma division of Gram-negative proteobacteria, including the two major plant-pathogenic genera Xanthomonas and Xylella. Many Xanthomonas and Xylella species cause important plant diseases that lead to enormous economic loss in agriculture. This application describes novel methods using genetic engineered bacteriophages to inhibit Xylella and Xanthomonas infection in the field.

X. fastidiosa causes several diseases of economic importance: grapevine Pierce's disease (PD); citrus variegated chlorosis; peach phony disease; plum leaf scald; as well as leaf scorch diseases on oleander, almond, coffee, pecan, amenity tree species, and olive quick decline syndrome (OQDS) that was recently identified.

Xylella fastidiosa is a xylem-limited fastidious (hard to culture) bacterium that can attack olive, grape, almond, plum and laurel trees among 309 plant species. X. fastidiosa multiplies and spreads slowly up and down the xylem of the tree from the site of infection. This organism was first identified in 1993 as the causal agent of citrus variegated chlorosis, a disease that affects varieties of sweet oranges. This disease was first noticed in Brazil in 1987, and it greatly affects commercial orchards resulting in crop devastation worldwide. Other strains of this species cause a range of diseases in mulberry, pear, almond, elm, sycamore, oak, maple, pecan and coffee, which collectively result in multi-million dollar devastation of economically important plants. The bacteria are transmitted from the gut of the insect vector (sharpshooter leafhopper) to the plant xylem when the insect feeds. Populations of X. fastidiosa restrict water movement in the xylem, but the true mechanisms involved in symptom manifestation remain unknown. Xylella fastidiosa produces a wide variety of pathogenic factors for colonization in a host-specific manner including a large number of fimbrial and afimbrial adhesins for attachment.

Xylella fastidiosa is the causal agent for the grapevine Pierce's disease since it was first discovered in 1892 in California. The disease is endemic in northern California, being vectored by the blue-green sharpshooter. It became a real threat to California's wine industry when the glassy-winged sharpshooter, native to the southeast United States, was discovered in California since 1996.

An epidemic of oleander leaf scorch was first noticed in 1994 in southern California. The disease seems to kill oleander within about two years of the first appearance of symptoms.

In Europe, X. fastidiosa has attacked olive trees (Olea europea) in the Salento area of Southern Italy causing the Olive Quick Decline Syndrome (OQDS). In mid-October 2013, the NPPO of Italy informed the EPPO Secretariat of the first detection of X. fastidiosa on its territory. Despite the fact that the cause of Xylella outbreak in Italy is still unclear (Abbot A, 2015), this disease is causing a rapid decline in olive plantations, and by April 2015 it was affecting the whole Province of Lecce and other zones of Apulia (Giampetruzzi, et al., 2015). The bacterium had never previously been confirmed in Europe. The disease's impact comes on top of a particularly bad year for Spanish and Italian olive growers in 2014 due to pests and the weather, with harvests in Italy down 40-50%.

Xanthomonas species causes many important plant diseases in fruit and vegetable fields, such as Xanthomonas campestris pv. campestris (Xcc) (black rot), X. fuscans (common blight of bean), X. campestris pv. vesicatoria (bacterial leaf spot on tomato and pepper), X. campestris pv. raphani (Leave spot disease), X. albilineans (sugarcane leaf scald) and, etc.

Black rot is considered the most important and destructive disease of crucifers (e.g. cabbage, broccoli, cauliflower, etc.), infecting all cultivated varieties of brassicas worldwide. Black rot was first reported in 1889 in US and has been found in nearly all countries in which vegetable brassicas are cultivated. Cabbage cultivation is a multi-billion dollar industry worldwide. In 2007, the cabbage crop in the US exceed $413 millions (1.4M+ tons) (USDA, 2008). Black rot pathogen Xcc spreads rapidly, and losses due to the disease may exceed 50% in warm, wet climates. The importance of using disease-free seed and/or transplants is highlighted by the fact that “as few as three infected seeds in 10,000 (0.03%) can cause black rot epidemics in a field” (http://ipm.illinois.edu/vegetables/diseases/black_rot/index.html). In transplant beds, an initial infection level of 0.5% can rise to 65% in just three weeks (William, 1980). The development and use of black rot resistant cultivars has long been recognized as an important method of control, but in practice has had limited success. Resistance to the most important pathogenic races of Xcc is rare in Brassica oleracea (e.g. cabbage, broccoli, cauliflower) (Taylor et al., 2002).

Xanthomonas campestris pv. vesicatora causes bacterial leaf spot (BLS) on peppers and tomatoes. BLS is a very economically important disease for tomato and pepper producers because it affects all above ground parts of the plant, including leaf spots, fruit spots and stem cankers. Plants can drop 50-100% of their foliage, which causes leaves fruit vulnerable to sunscald, further reducing yield.

The common blight of bean (CBB), caused by Xanthomonas fuscans, is the most devastating bacterial disease of bean and one of the five major diseases of bean. It causes significant yield loss that can exceed 40%, and seed quality losses impact not only bean production but also seed industry worldwide. The wide geographical distribution of CBB is presumed to be due to an efficient seed transmission. CBB affects seed and pod production and marketability of common bean (Phaseolus vulgaris L.) but also lima bean (P. lunatus L.), tepary (P. acutifolius A. Gray), scarlet runner bean (P. coccineus L.), and several species belonging to Vigna.

Xanthomonas albilineans is a systemic, xylem-invading pathogen that causes leaf scald of sugarcane (Saccharum interspecific hybrids). This disease occurs in at least 66 countries in the world and can cause severe yield losses.

Cassava is an essential source of food and income for hundreds of millions of people in many tropical countries. This major crop is threatened by bacterial pathogens, the vascular and systemic infection of Xanthomonas phaseoli pv. manihotis (Xpm) and Xanthomonas cassavae has received considerable attention due to its devastating potential in the tropics and scientific importance worldwide (Zárate-Chaves, 2021).

Taken together, the current treatment of plant bacterial diseases caused by Xylella or Xanthomonas is either lack or very limited to chemical treatment, such as spraying a mixture of copper and mancozeb, which prevents further infection. Chemical methods kill a variety of pathogens present at the time of spraying, but they also leads to the potential problem of contamination of heavy metals in the environment or change microenvironment of microbes. New method is desperately required to eradicate these bacterial pathogens that lead to economic crop loss worldwide.

Bacteriophage (or called “phage” in this application interchangeably) is a virus that that infects and replicates within bacteria. In the effort to engineer bio-control phages that inhibit or eradicate Xylella or Xanthomonas pathogens, here we engineered new recombinant phages, including Xanthomonas phages (phiXfu, Xfv, Xfr, fXa, and phiXca), and Xylella phages (fXy1, fXy2, fXy3, fXy4, fXy5, fXy6, fXy7, fXy8, fXy9, and fXy10). The small genome size of these filament phages (6,062-8,520 base pair) and their host ranges make them novel tools to infect Xylella or Xanthomonas bacteria.

This invention includes applications of these newly engineered phages (fXy1, fXy2, fXy3, fXy4, fXy5, fXy6, fXy7, fXy8, fXy9, fXy10, phiXfu, Xfv, Xfr, fXa, phiXca) (SEQ ID NO: 1-15) as bio-control agents to eradicate plant diseases caused by various Xylella or Xanthomonas pathogens.

### BRIEF DESCRIPTION OF THE FIGURES

FIG. 1: Electron micrographs of new Xanthomonas and Xylella phages stained with 1% uranyl formate: (A) phiXfu, (B) Xfv, (C) Xfr, (D) fXa, (E) phiXca, (F) fXy1, (G) fXy2, (H) fXy3, (I) fXy4, (J) fXy5, (K) fXy6, (L) fXy7, (M) fXy8, (N) fXy9, (0) fXy10.

FIG. 2: Engineered Xyllela and Xanthomonas phages integration into host bacterial chromosome. (A) Cartoon illustration of phage integration into host bacterial genome via their phage attachment sites (attP) and host chromosome attB. PCR primers (arrows) were designed to flank ˜400 bp region at both sides of attR after integration. (B) PCR DNA fragments were amplified from purified bacterial genomic DNA and primers as (A) at 16 hours post-infection, and analyzed in 1% Tris-acetic acid-EDTA agarose gel. Lane 1: phiXfu, lane 2:Xfv, lane 3:Xfr, lane 4:fXa, lane 5:phiXca, lane 6:fXy1, lane 7:fXy2, lane 8:fXy3, lane 9:fXy4, lane 10:fXy5, lane 11:fXy6, lane 12:fXy7, lane 13:fXy8, lane 14:fXy9, lane 15:fXy10, lane 16:fXy1, lane 17:fXy2. White lines separate results from 2 areas in the same gel, and the data are combined to make a single representative figure. M is DNA molecular marker.

FIG. 3. The inhibitory effect of Xylella phages on bacterial growth. (A) Examples of the growth curve of Xylella bacteria (colony forming unit/ml) within 24 hours post-infection with fXy1 and fXy2 phage (SEQ ID NO: 1 and 2). (B) The summary table of the inhibitory effect of fXy phages (SEQ ID NO: 1 to 10) on the growth of Xylella fastidiosa bacteria in the phage-infected cultures at 48 hours post-infection. The number in this table refers to the percentage of (colony forming unit/ml) compared to non-infected culture. The inhibitory effect >80% is labeled in bold (p<0.01, Student t-test). Data are from three independent experiments each.

FIG. 4. The summary table of the inhibitory effect of phiXfu, Xfv, Xfr, fXa, and phiXca phages (SEQ ID NO: 11 to 15) on the growth of Xanthmonas bacteria in the phage-infected cultures at 48 hours post-infection. The number in this table refers to the percentage of (colony forming unit/ml) compared to non-infected culture. The inhibitory effect >80% is labeled in bold (p<0.01, Student t-test). Data are from three independent experiments each.

FIG. 5. Examples of Xylella bacteria eradicated by Xylella phages. (A) Xylella bacteria were infected with or without fxy1 and fxy2 phages in the culture medium for 24 hours. Bacterial viability was assessed using the propidium iodide in the dark at room temperature for 25 min. Flow cytometric measurements were performed on FACSArray Bioanalyzer (BD). The percentage of dead bacteria stained with propidium iodide were shown in “not(P1)”. (B) The summary table of cell death rate of Xylella bacteria (% of total bacterial cells, N=20,000) caused by fXy1, fXy2, fXy3, fXy4, fXy5, fXy6, fXy7, fXy8, fXy9, and fXy10 phage (SEQ ID NO: 1 to 10) at 24 hours post-infection as (A). Death rate >20% are labeled in bold (p<0.01, Student t-test). Data are from three independent experiments each.

FIG. 6. The summary table of cell death rate of Xanthomonas bacteria (% of total bacterial cells, N=20,000) caused by phiXfu, Xfv, Xfr, fXa, and phiXca phages (SEQ ID NO: 11 to 15) at 24 hours post-infection. Flow cytometric measurements were performed as FIG. 5. Death rate >20% are labeled in bold (p<0.01, Student t-test). Data are from three independent experiments each.

### DETAILED DESCRIPTION

In an aspect, the disclosure provides for a composition, seed, plant, vector, or construct comprising a sequence described herein.

The nucleic acid sequences described herein are useful in controlling and eradicating plant Xanthomonas and Xylella pathogens associated with plant bacterial pathogens, such as olive quick decline syndrome (OQDS), Pierce's disease (grape), phoney peach disease, bacterial leaf scorch, oleander leaf scorch, citrus variegated chlorosis disease, black rot, bacterial blight, bacterial leaf spot, Bacterial blight of bean, sugarcane leaf scald, cassava bacterial blight, etc.

In another aspect, the disclosure provides for methods of utilizing a sequence described herein to inhibit or eradicate pest activity. In yet another aspect, the disclosure provides for methods of utilizing a sequence described herein to treat or reduce pest activity, for example, pest activity in Xanthomonas and Xylella, for example, Xanthomonas albilineans, Xanthomonas arboricola sp., Xanthomonas axonopodis sp., Xanthomonas bromi, Xanthomonas campestris spp., Xanthomonas campestris pv. raphani, Xanthomonas campestris pv. campestris, Xanthomonas cassavae, Xanthomonas citri, Xanthomonas codiaei, Xanthomonas cucurbitae, Xanthomonas cynarae, Xanthomonas fragariae, Xanthomonas fuscans, Xanthomonas gardneri, Xanthomonas hortorum, Xanthomonas hyacinthi, Xanthomonas melonis, Xanthomonas oryzae sp., Xanthomonas pisi, Xanthomonas populi, Xanthomonas sacchari, Xanthomonas theicola, Xanthomonas translucens sp., Xanthomonas vasicola, Xanthomonas vesicatoria, Xanthomonas spp., Xylella fastidiosa, Xylella taiwanesis, Xylella sp. CFBP8070, Xylella sp. CL3.52, Xylella sp. PLS194, Xylella sp. PLS2, Xylella sp. PLS222, Xylella sp. PLS45, Xylella sp. WO2006108179, but also including other bacteria such as Pseudomonas and Enterobacteriaceae infection.

In an aspect, the disclosure provides for a method of preventing, inhibiting, reducing, or treating infection or infestation of olive quick decline syndrome (OQDS), Pierce's disease (PD), and citrus variegated chlorosis disease (CVC), leaf spot disease, sugarcane leaf scald, phoney peach disease, bacterial leaf scorch, oleander leaf scorch, black rot, and/or bacterial leaf spot, and other Xanothomonas or Xylella infection.

In an aspect, nucleotide sequences encoding the proteins of the present invention include the sequence set forth in SEQ ID NO: 1 to 15 and variants, fragments, and complements thereof. In another aspect, proteins of the present invention are encoded by a nucleotide sequence sufficiently identical to the nucleotide sequence of SEQ ID NO: 1 to 15. By “sufficiently identical” is intended an amino acid or nucleotide sequence that has at least about 50%, 60%, or 65% sequence identity, about 70% or 75% sequence identity, about 80% or 85% sequence identity, about 90%, 91%, 92%, 93%, 94%, 95%, 96%, 97%, 98%, 99% or greater sequence identity compared to a reference sequence using one of the alignment programs described herein using standard parameters. One of skill in the art will recognize that these values can be appropriately adjusted to determine corresponding identity of proteins encoded by two nucleotide sequences by taking into account codon degeneracy, amino acid similarity, reading frame positioning, and the like.

Variants include a nucleic acid molecule that hybridizes to the nucleic acid molecule of SEQ ID NO: 1 to 15 or a complement thereof, under the condition that permit a formation of a nucleic acid duplex at the temperature from 20° C.-29° C. below the melting temperature of the nucleic acid duplex in the hybridization and washing solution with 0.165-0.33 molar concentration of sodium chloride. Variant proteins include polypeptides encoded by SEQ ID NO: 1 to 15 that differ in amino acid sequence due to mutagenesis. Variant proteins encompassed by the present invention are biologically active, that is they continue to possess the desired biological activity of the native protein, that is, retaining pesticidal activity. In some embodiments, the variants have improved activity relative to the parental sequences used for mutagenesis.

As described in the sequence listing, SEQ ID NO: 1 comprises a nucleotide sequence of recombinant fXy1 phage.

As described in the sequence listing, SEQ ID NO:2 comprises a nucleotide sequence of recombinant fXy2 phage.

As described in the sequence listing, SEQ ID NO:3 comprises a nucleotide sequence of recombinant fXy3 phage.

As described in the sequence listing, SEQ ID NO:4 comprises a nucleotide sequence of recombinant fXy4 phage.

As described in the sequence listing, SEQ ID NO:5 comprises a nucleotide sequence of recombinant fXy5 phage.

As described in the sequence listing, SEQ ID NO:6 comprises a nucleotide sequence of recombinant fXy6 phage.

As described in the sequence listing, SEQ ID NO:7 comprises a nucleotide sequence of recombinant fXy7 phage.

As described in the sequence listing, SEQ ID NO:8 comprises a nucleotide sequence of recombinant fXy8 phage.

As described in the sequence listing, SEQ ID NO:9 comprises a nucleotide sequence of recombinant fXy9 phage.

As described in the sequence listing, SEQ ID NO:10 comprises a a nucleotide sequence of recombinant fXy10 phage.

As described in the sequence listing, SEQ ID NO: 11 comprises a nucleotide sequence of recombinant phiXfu phage.

As described in the sequence listing, SEQ ID NO:12 comprises a nucleotide sequence of recombinant Xfv phage.

As described in the sequence listing, SEQ ID NO: 13 comprises a nucleotide sequence of recombinant Xfr phage.

As described in the sequence listing, SEQ ID NO:14 comprises a nucleotide sequence of recombinant fXa phage.

As described in the sequence listing, SEQ ID NO:15 comprises a nucleotide sequence of recombinant phiXca phage.

It is recognized that DNA sequences may be altered by various methods, and that these alterations may result in DNA sequences encoding proteins with amino acid sequences different than that encoded by a pesticidal protein of the present invention. This DNA may be altered in various ways including substitutions, deletions, truncations, and insertions of one or more nucleotides of SEQ ID NO: 1 to 15 including up to about 1, about 2, about 3, about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, about 100 or more nucleotide substitutions, deletions or insertions.

The skilled artisan will further appreciate that changes can be introduced by mutation of the nucleotide sequences of the invention thereby leading to changes in the amino acid sequence of the encoded phage proteins, without altering the biological activity of the proteins. Thus, variant isolated nucleic acid molecules can be created by introducing one or more nucleotide substitutions, additions, or deletions into the corresponding nucleotide sequence disclosed herein, such that one or more amino acid substitutions, additions or deletions are introduced into the encoded protein. Mutations can be introduced by standard techniques, such as site-directed mutagenesis and PCR-mediated mutagenesis. Such variant nucleotide sequences are also encompassed by the present invention.

For example, conservative amino acid substitutions may be made at one or more, predicted, nonessential amino acid residues. A “nonessential” amino acid residue is a residue that can be altered from the wild-type sequence of a pesticidal protein without altering the biological activity, whereas an “essential” amino acid residue is required for biological activity. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

In an aspect, amino acid substitutions may be made in nonconserved regions that retain function. In general, such substitutions would not be made for conserved amino acid residues, or for amino acid residues residing within a conserved motif, where such residues are essential for protein activity. Examples of residues that are conserved and that may be essential for protein activity include, for example, residues that are identical between all proteins contained in an alignment of similar or related toxins to the sequences of the invention (e.g., residues that are identical in an alignment of homologous proteins). Examples of residues that are conserved but that may allow conservative amino acid substitutions and still retain activity include, for example, residues that have only conservative substitutions between all proteins contained in an alignment of similar or related toxins to the sequences of the invention (e.g., residues that have only conservative substitutions between all proteins contained in the alignment homologous proteins). However, one of skill in the art would understand that functional variants may have minor conserved or nonconserved alterations in the conserved residues.

Unless otherwise specified, “a” or “an” means “one or more”. As used herein, the terms “and” and “or” may be used to mean either the conjunctive or disjunctive. That is, both terms should be understood as equivalent to “and/or” unless otherwise stated. As used herein, unless otherwise stated for a particular parameter, the term “about” refers to a range that encompasses an industry-acceptable range for inherent variability in analyses or process controls, including sampling error. Consistent with the Model Guidance of AAFCO, inherent variability is not meant to encompass variation associated with sloppy work or deficient procedures, but, rather, to address the inherent variation associated even with good practices and techniques.

As used herein, the term “coating” means a partial or complete covering that covers at least a portion of a surface, for example a surface of a food, plant, seed or fruit. In one example, a food, plant, seed or fruit may be partially covered with a coating such that only part of the plant is covered, and part of the plant is not covered and is thus exposed. In another example, the plant may be completely covered with a coating such that the entire plant is covered and thus not exposed. Thus a coating may cover from a negligible amount up to the entire surface. A coating can also be coated onto other coatings such that a layering of coatings can be present. For example, a plant can be coated with coating A, and coating A can be coated with coating B, such that coating A and coating B each form a layer.

As used herein, the terms “bacteriophage” and “phage” are used interchangeable and refer to a virus which is lytic or otherwise harmful to bacteria of one or more undesirable strains, or which is lysogenic and integrates its DNA material into host bacterial genome. Undesirable bacteria may be or may produce compounds which are potentially pathogenic for plants, or may be associated with spoilage, malodor, aesthetic decline, or other deterioration of a food product colonized by the undesirable bacteria. As used herein, “bacterium”, “bacteria” or “target bacterium” refers to an undesirable micro-organism susceptible to infection and lysis, apoptosis, or alternate modes of cell death caused by a bacteriophage. Different strains of bacteriophage may infect different strains of bacteria with different results, or may infect some strains of bacteria but not others.

As used herein, “isolated” will mean material removed from its original environment (e.g., the natural environment in which the material occurs) or liquid culture, and thus is “altered by the hand of man” from its natural environment. Isolated material may be, for example, foreign nucleic acid included in a vector system, foreign nucleic acid contained within a host cell, or any material which has been removed from its original environment and thus altered by the hand of man. Isolated material further encompasses isolated phage or particular bacterial isolates of Xanthomonas or Xylella, isolated and cultured separately from the environment in which it was located, where these isolates are present in purified compositions that do not contain any significant amount of other bacteriophage or bacterial strains, respectively. As used herein, “significant” will mean an amount of a substance present in the total measured composition, wherein the substance is present in greater than 1% of the total volume or concentration of the composition.

As used herein, “recombinant”, “variants”, and “recombinant variants” will mean genomic materials (nucleotides, DNA, RNA, proteins) are artificially engineered and have the same nucleotide and/or amino acid sequences where one or more residues are added, deleted, or substituted.

As used herein, “pv.” means pathovar, a type of classification based on the host plant that is attacked by Xanthomonas campestris.

As used herein, “Xcc” means “Xanthomonas campestris. pv. campestris”.

As used herein, “colonization” or “colonized” will refer to the presence of Xylella fastidiosa, Xylella taiwanesis, Xylella sp. CFBP8070, Xylella sp. CL3.52, Xylella sp. PLS194, Xylella sp. PLS2, Xylella sp. PLS222, Xylella sp. PLS45, Xylella sp. WO2006108179, Xcc, Xanthomonas citri, Xanthomonas albilineans, Xanthomonas arboricola sp., Xanthomonas axonopodis sp., Xanthomonas bromi, Xanthomonas campestris spp., Xanthomonas cassavae, Xanthomonas codiaei, Xanthomonas cucurbitae, Xanthomonas cynarae, Xanthomonas fragariae, Xanthomonas fuscans, Xanthomonas gardneri, Xanthomonas hortorum, Xanthomonas hyacinthi, Xanthomonas melonis, Xanthomonas oryzae sp., Xanthomonas pisi, Xanthomonas populi, Xanthomonas sacchari, Xanthomonas theicola, Xanthomonas translucens sp., Xanthomonas. vasicola, Xanthomonas campestris pv. vesicatoria, Xanthomonas campestris pv. raphani, Pseudomonas and Enterobacteriaceae on a plant, foodstuff or environmental surface without perceptible significant alteration to that plant, foodstuff or surface other than the presence of bacteria. The terms “colonization” and “colonized” stand in contrast to the terms “infection” or “infected” which are commonly understood to require perceptible deleterious alteration as part of their definition. “Colonization” and “colonized” may also refer to the presence of bacteria in or on a plant without perceptible damage, alteration, or disease.

As used herein, “ATCC” will mean the “American Type Culture Collection”, which is located at 10801 University Boulevard, Manassas, Virginia, 20110-2209, USA.

“Centrifugation”, the name given to separation applications which involve spinning around an axis to produce a centrifugal force, is a way to increase the magnitude of the gravitational field. The particles or materials (such as viruses, phages, DNA, etc.) in suspension experience a radial centrifugal force moving them away from the axis of rotation. The radial force generated by the spinning rotor is expressed relative to the earth's gravitational force and herein expressed as “g-force” (with g from “gravitational”).

As used herein, “homology” will mean the degree of similarity between two nucleic acids (based on comparison of the chemical structure of the nucleic acids, as expressed by the sequence of nucleotides making up the nucleic acid or biologic function, as determined by whether two nucleic acids of minimum length 500 nucleotides and maximum length 10,000 nucleotides will hybridize to form a double-stranded complex).

As used herein, “purify” will mean a method to generate a macromolecule essentially free of any similar macromolecules that would normally be found with it in nature. In other words, a purified protein is in a composition that contains no more than 1% other protein from the same taxonomic species. A purified composition excludes media components, recipients or other non-contaminating compounds resulting from culturing, processing or formulating the composition.

As used herein, “amplification” will mean the in vitro production of multiple copies of a particular nucleic acid sequence. The amplified sequence is usually in the form of DNA. A variety of techniques for carrying out such amplification are described in a review article (Van Brunt, 1990). Polymerase chain reaction (PCR) is a prototype of nucleic acid amplification, and use of PCR herein should be considered exemplary of other suitable amplification techniques.

A regular method of quantification by culturing and counting includes a technique which is typically referred to as a “plaque assay”. In plaque assays, the phages that are to be quantified are mixed with a known concentration of host bacterial cells and transferred to a liquid (e.g., buffer, mineral salts diluent, or broth). The mixture is then transferred to a semisolid growth medium. The concentration of host cells must be sufficiently great to form a confluent layer, which is typically referred to as a “lawn,” in the semisolid growth medium as the cells grow. During incubation of the phage-bacteria mixture, many of the viable viruses infect host cells. Subsequently, new viruses are produced within infected host cells, which are eventually destroyed, or “lysed,” so that new viruses may be released. The new viruses then attack and eventually lyse cells that are adjacent to host cells from which the new viruses were released. This spread of infection, which continues as long as host cells are metabolizing, results in formation of clear areas, which are typically referred to as “plaques,” in the host cell lawn. The number of viruses that were present in the original mixture is determined by counting the number of plaques that are formed in the host cell lawn. Accordingly, viruses that are quantified by this method are referred to as “plaque-forming units” (“PFU”). The term “colony-forming unit” (“CFU”) is an estimate of viable bacterial numbers. The appearance of a visible colony requires significant growth of the initial cells plated—at the time of counting the colonies it is not possible to determine if the colony arose from one cell or 1,000 cells. Therefore, the results are given as CFU/mL (colony-forming units per milliliter) for liquids, and CFU/g (colony-forming units per gram) for solids to reflect this uncertainty (rather than cells/mL or cells/g).

As used herein, the “lytic” cycle means bacteriophage replication results in the destruction of the infected cell and its membrane. In the lytic cycle, the viral DNA exists as a separate molecule within the bacterial cell, and replicates separately from the host bacterial DNA. In contrast, the “lysogenic” cycle used herein is characterized by integration of the bacteriophage nucleic acid into the host bacterium's genome or formation of a circular replicon in the bacterium's cytoplasm. In this condition the bacterium continues to live and reproduce normally.

As used herein, the “multiplicity of infection” (“MOI”) is the ratio of infectious agents (e.g. phage or virus) to infection targets (e.g. cell or bacteria). For example, when referring to a group of bacterial cells inoculated with infectious phage particles, the multiplicity of infection or MOI is the ratio of the number of infectious virus particles to the number of target cells present in a defined space.

As used herein, “cell death” will mean bacteria is killed by bacteriophages and lose the ability to replication, growth, or infection.

Xyllela and Xanthomonas phages have binding specificity to the host bacteria, and are capable of lysing infected host cells. Xyllela and Xanthomonas phages can also grow through lysogenic cycle by integrating phage DNA into host genome. The invention further contemplates “variants” or “strains” of Xyllela and Xanthomonas phages, which are phages having variation(s) less than 10% differences in the genomic sequences and polypeptides encoded thereby while retaining the same or different general genotypic and phenotypic characteristics as the recombinant Xyllela and Xanthomonas phage (SEQ ID: 1 to 15). The invention also contemplates “recombinant” Xyllela and Xanthomonas phages, which have modified genotypic or phenotypic characteristics relative to the filamentous phages harboring genes encoding novel phenotypic traits. Such recombinant Xyllela and Xanthomonas phages are engineered to contain mutations or lose genes having traits not found in natural sequences. If not specified, “recombinant phages” will mean genomic materials (nucleotides, DNA) of phages are artificially engineered and have the same nucleotide and amino acid sequences where one or more residues are added, deleted, or substituted.

As used herein, “Xylella phages” in this invention means recombinant fXy1, fXy2, fXy3, fXy4, fXy5, fXy6, fXy7, fXy8, fXy9, and fXy10 phages (SEQ ID NO: 1 to 10) and their “variants”, including “Xylella phage mutants” occurring naturally or induced by or chemical mutagens.

As used herein, “Xanthomonas phages” in this invention means recombinant phiXfu, Xfv, Xfr, fXa, and phiXca phages (SEQ ID NO: 11 to 15), including “Xanthomonas phage variants” in this invention will include “Xanthomonas phage mutants” occurring naturally or induced by chemical mutagens.

### EXAMPLES

### Example 1: Growth and Purification of Xanthomonas and Xylella Phages from Bacteria

Propagation of bacteria and recombinant Xanthomonas and Xylella phages is performed as described as the following, which are each hereby incorporated by reference in their entireties (Tseng, et al, 1990; Lin et al., 1994; Lopes and Torres, 2006). Xylella fastidiosa, Xcc, X. campestris pv. vesicatoria, X. fuscans, and X. campestris pv. raphani, were purchased from ATCC (Manassas, VA) and used as bacterial host for cultivation of Xanthomonas and Xylella phages and their variants. Spot tests and plaque assays were carried out as described previously (Tseng et al., 1990) using culture supernatants as phage sources and each of the strains as an indicator host. Xanthomonas bacteria was grown in TYG medium, which contained, per liter: 10 g tryptone, 6 g yeast extract, 1 mM MgSO₄ and 5 g glucose. Xylella fastidiosa was grown in phosphate-buffered charcoal-yeast extract (PBCY) medium, which contained, per liter: 1.0 g KH₂PO₄, 1.1 g K₂HPO₄, pH 6.9, 0.25 g FeSO₄7H₂O, 0.4 g MgSO₄7H₂O, 2 g activated charcoal, 10 g yeast extract, 10 g agar or 8 g Gelrite. (Lopes and Torres, 2006). The olive strains of Xylella fastidiosa (strain CodiRO or Pauca and Salento) were isolated from olive leaves exhibiting OQDS in PBCY medium with 200 microgram/ml natamycin. In plaque assays (Eisensfork, 1967), using a TYG or PBCY double layer, phages formed turbid plaques of about 1 mm in diameter on a lawn of bacteria, after 12 h at 28° C. Phages are harvested from semi-confluent lysis on double-layer TYG or PBCY plates. Distilled water or buffer is added to the plates, which are kept overnight in a cold room. The phage solution is collected and the cell debris is removed by centrifugation at 10000 g-force for 30 min at 4° C. The phage titer is assayed by the double-layer agar method. The phage is stored at 4° C. for use.

DNAs containing Xanthomonas and Xylella phage sequences (SEQ ID NO: 1 to 15) were synthesized directly by a company (GenScript, Coralville, Iowa). The synthesized DNA fragments are ligated and subcloned into a kanamycin-resistant pUC57 vector and amplified in E. coli.

The method to transform artificially synthesized DNAs into Xanthomonas cells is performed as the following (Tseng et al., 1990), which is herein incorporated by reference in its entirety. To prepare Xanthomonas competent cells for PEG-mediated transformation, an overnight bacterial culture was diluted 80-fold into 40 ml of TYG and grown until OD₅₅₀ was 0.5; the cells were then harvested by centrifugation and suspended in 10 ml of cold TC buffer (0.25 M-Tris-HCl pH 7.2, containing 0.1 M CaCl₂)), then incubated on ice for 10 min. The suspension was centrifuged again and the pellet was resuspended in 1 ml of cold TC buffer and kept on ice until use. The transfection mixture (400 microliter) contained 100 [symbol] l of competent cells, 0.5 microgram synthesized DNA (or 1.0 microgram phage ssDNA described later), 150 microliter of 40% PEG 6000 in 0.1 M Tris-HCl pH 7.2 and sterile distilled water. The mixture was incubated on ice for 30 rain followed by heat shock at 30° ° C. for 2 min. After heat shock, 3.6 ml of TYG broth was added immediately and the mixture was incubated at 28° C. with shaking for 30 min. Transfected bacteria were counted as infective centres by double-layer plaque assay.

Preparation of electrocompetent Xylella fastidiosa cells, and the electroporation method to transform DNA into Xylella bacteria were performed as the following (Guilhabert et al., 2001), which is herein incorporated by reference in its entirety. The bacterial cells were initially plated on PBCY agar plates. After incubating for 10 to 12 days at 28° C., a piece of agar medium containing X. fastidiosa cells was cut from a plate and used to inoculate 35 ml of liquid PBCY medium. These liquid cultures were allowed to grow for 7 to 10 days at 28° C.; then the cell density was adjusted to 10⁶ cells per ml (optical density at 600 nm of 0.0025) with fresh PBCY medium. Adjusted cell culture (100 microliter) was used to inoculate each of six PBCY agar plates. After 6 days at 28° C., the cells were gently washed off each plate with 2 to 3 ml of PBCY medium. The cells were harvested by centrifugation (5,000×g-force) at 4° C. for 5 min, washed in 10 ml of cold, sterile 10% glycerol, concentrated by centrifugation, and suspended in 1 ml of cold 10% glycerol. The suspension was centrifuged at 5,000×g-force for 5 min at 4° C., resuspended in 10% glycerol to a final concentration of approximately 10⁹ cells per ml, and held on ice until electroporated with the DNAs containing SEQ ID NO: 1 to 10.

The method to electroporate artificially recombinant phage RF DNAs into Xylella cells is performed as the following, which is herein incorporated by reference in its entirety. Aliquots (20 microliter) of concentrated, electrocompetent Xylella cells were transferred to a cold, 1.5-ml microcentrifuge tube containing 0.1 microgram of DNA. The cell-DNA mixture was placed between the chilled electrodes of an electroporation cuvette (0.1-cm gap; BTX, Holliston, MA, U.S.A.) and subjected to a single, high-voltage pulse. Pulses were generated and delivered with the ECM 630 Electro Cell Manipulator (BTX, Holliston, MA, USA). An electric field of 10 kV per cm for 5 mini-second, with a resistance value of 4,000 ohm and a capacitance of 330 microF, was applied across the cell-DNA suspension. After the pulse delivery, the cells were immediately removed from the electroporation chamber and inoculated into 1 milliliter of liquid PBCY medium without antibiotics. The cells were incubated for 24 h at 28° C. with constant shaking (100 rpm). The 1 ml of liquid PBCY medium containing the electroporated sample was plated entirely on 10 plates of selective PBCY medium.

Preparation of electrocompetent Xanthomonas cells, and the electroporation method to transform DNA into Xanthomonas bacteria is performed as the following (Yang et al., 1991; Wang and Tseng, 1992), which are herein incorporated by reference in its entirety. A single colony of host Xanthomonas bacteria was inoculated into 3.0 ml of TYG in a 15 ml vial and grown overnight. Then 2.0 ml of the overnight culture was transferred into a flask containing 200 ml of fresh TYG and grown until an OD₅₅₀ of 0.3-0.4 was attained. The cells were harvested by centrifugation (8,000×g-force) at 4° C. and washed five times with 20 ml of cold sterile deionized water. Then the cells were resuspended in 200-300 microliter sterile deionized water to obtain a final concentration of approximately 1.1×10¹¹ cells/ml, kept on ice and used directly for electroporation.

The method to electroporate recombinant Xanthomonas phage RF DNAs into Xanthomonas cells is performed as the following, which is herein incorporated by reference in its entirety. Forty microliter of the washed Xanthomonas cells (ca 4.5×10⁹ cells) were added to a prechilled electroporation cuvette (0.1-cm gap; BTX, Holliston, MA, U.S.A.), followed by the addition of 0.1 microgram DNA in 1 microliter. Then the mixture was exposed to a field strength of 2.5 kV with a 25 microF capacitor and a 400 ohm resistor. After the pulse, 1 ml of TYG medium was added to the mixture. For transformation by DNAs containing SEQ ID NO: 11 to 15, the mixture was used to assay for infective centres on the double layer. When transformed by plasmids, the mixture was incubated at 28° C. before plating out for colony counting.

Phages are purified as described in (Kuo et al., 1987, Tseng, 1990), which is hereby incorporated by reference in its entirety. An overnight infected bacterial culture is harvested, and host cells and debris is removed by centrifugation at 6000 g-force for 10 min. Solid NaCl is added to the supernatant to a final concentration of 0.5 M, and polyethyene glycol 6000 is then added to a final concentration of 3%. After thorough stirring, the mixture is allowed to settle overnight in a cold room. The precipitate is collected by centrifugation at 6000 g-force for 10 minutes and then resuspended in distilled H₂O. Further purification was carried out by centrifugation through a CsCl step density gradient in a Beckman SW41 swinging bucket rotor at 23,000 rpm for 22 hours at 5° C.

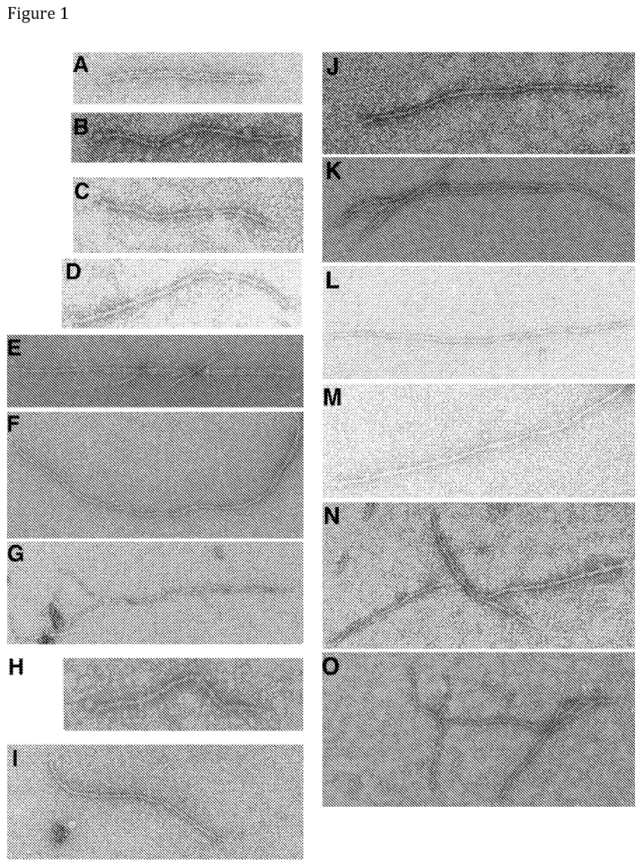

Results: Production of the Xanthomonas and Xylella phages. Purified phage particles were stained with uranyl formate and visualized with electron microscopy, and the morphology is shown in FIG. 1 [(A) phiXfu, (B) Xfv, (C) Xfr, (D) fXa, (E) phiXca, (F) fXy1, (G) fXy2, (H) fXy3, (I) fXy4, (J) fXy5, (K) fXy6, (L) fXy7, (M) fXy8, (N) fXy9, and (O) fXy10]. The phage particles were filamentous in shape with size ranging from 1000 (+200)×8 nm to 1120 (+200)×8 nm. These results confirm that Xylella and Xanthomonas bacteria transformed or electroporated by recombinant phage RF DNAs containing SEQ ID NO: 1 to 15 are able to generate the progeny Xylella and Xanthomonas phage particles.

### Example 2: Purification of Replication Form (RF) and Phage DNA of Recombinant Xanthomonas and Xylella Phages, and Bacterial Genome DNA

Preparation of the RF DNAs of Xanthomonas and Xylella phages from host bacteria are performed as described (Yang and Kuo, 1984), which are each hereby incorporated by reference in their entireties. Xanthomonas or Xylella bacteria are grown in 500 ml TYG or PBCY medium at 28° C. The cells, at a density of 2×10⁹ per ml, are infected with phages at a multiplicity of 20 and treated with 170 microgram/ml chloramphenicol at 10 min post-infection. After 4 hours of incubation at 28° C., the infected bacterial cells are harvested, chilled, washed once with 250 ml buffer (10 mM-Tris-HCl pH 8.0, 0.1 mM-EDTA), and RF DNAs are purified by Qiagen Plamid Maxi kit (Qiagen, Germantown, MD, USA). The RF DNA thus prepared is used directly for restriction endonuclease digestion or PCR. Digestion of RF DNA with restriction endonucleases, gel electrophoresis, DNA sequencing, and purification of restriction DNA fragments are performed as described in (Yang and Kuo, 1984), which is hereby incorporated by reference in its entirety.

Phage DNAs are purified as described in (Kuo et al., 1987), which is herein incorporated by reference in its entirety. A purified phage suspension is dialyzed against TEN (25 mM Tris-HCl; 10 mM EDTA and 0.15 M NaCl, pH 8.5). The phage coat protein is then dissociated by 2% sodium dodecyl sulfate (SDS) then further digested with Pronase (1 mg/ml) at 60° C. for 18 hour or overnight. Contaminating RNA is digested by the addition of RNase A (50 [symbol] g/ml) and incubated at 37° C. for 1 hour. The phage DNA was purified by TEN-saturated mixture of phenol:isoamyl alcohol:chloroform (25:1:24) and precipitated with ethanol.

To isolate genomic DNA from bacterial cells, bacterial cells at 2 to 4×10⁹ cells/ml are harvested by centrifugation, washed with 5 ml of 100 mM Tris-HCl, 10 mM NaCl at pH 7.6, and suspended in 1.8 ml of 50 mM Tris-HCl (pH 7.6) 100 mM NaCl, and 5 mM disodium EDTA. The genomic DNAs are purified using QIAamp DNA Kits (Qiagen, Germantown, MD, USA). Digestion of phage or bacterial genomic DNAs with restriction endonucleases, agarose gel electrophoresis, and DNA sequencing are performed (Kuo et al., 1987), which are hereby incorporated by reference in their entireties.

To verify Xanthomonas and Xylella phage DNA integration into bacterial chromosome genome, PCR was performed to generate about 400 base pair DNA fragments using 1 micro gram of bacterial genomic DNAs as the template and the primers at both sides of integrated attR sites. PCR DNA fragments were analyzed by 1% agarose gel electrophoresis.

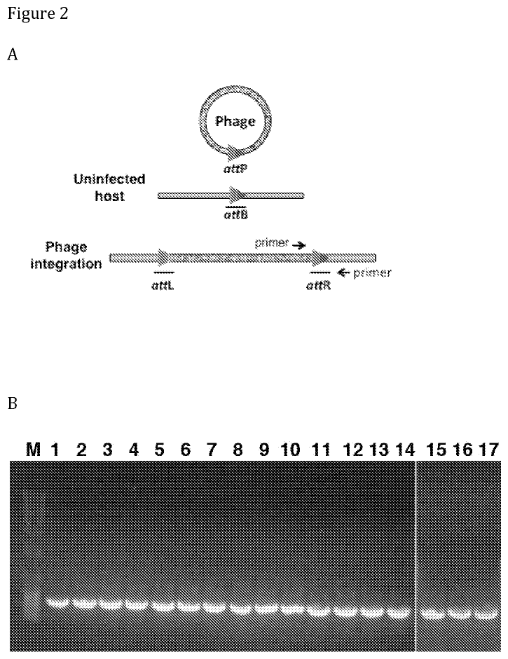

Results: Integration of Xanthomonas and Xylella phage DNAs into bacterial chromosome. The cartoon illustration of Xanthomonas and Xylella phage DNA integration into the bacterial host genome at 16 hour post-infection is shown in FIG. 2A (“Phage integration”). PCR analysis using the primers that flank both sides of phage-integrated sites attR confirms that (1) Xanthomonas and Xylella phages are able to infect their hosts; (2) phage DNAs can insert into their host bacterial genomes within 16 hours (FIG. 1B).

### Example 3: Xanthomonas and Xylella Bacterial Growth after Phage Infection

The growth curve for bacteria after Xanthomonas and Xylella phage infection is determined as the following (Kuo et al., 1987; Tseng et al., 1990) which is herein incorporated by reference in its entirety. Phage-infected bacteria (MOI of 10) are suspended in TYG or PBCY at a concentration of 1×10⁸ cells/ml and incubated at 28° C., with shaking. At different intervals, samples are diluted and spread on TYG or PBCY agar plates. After colony formation the number of colonies is counted. The respective cell types are determined and calculated as percent of total population of non-infected bacteria.

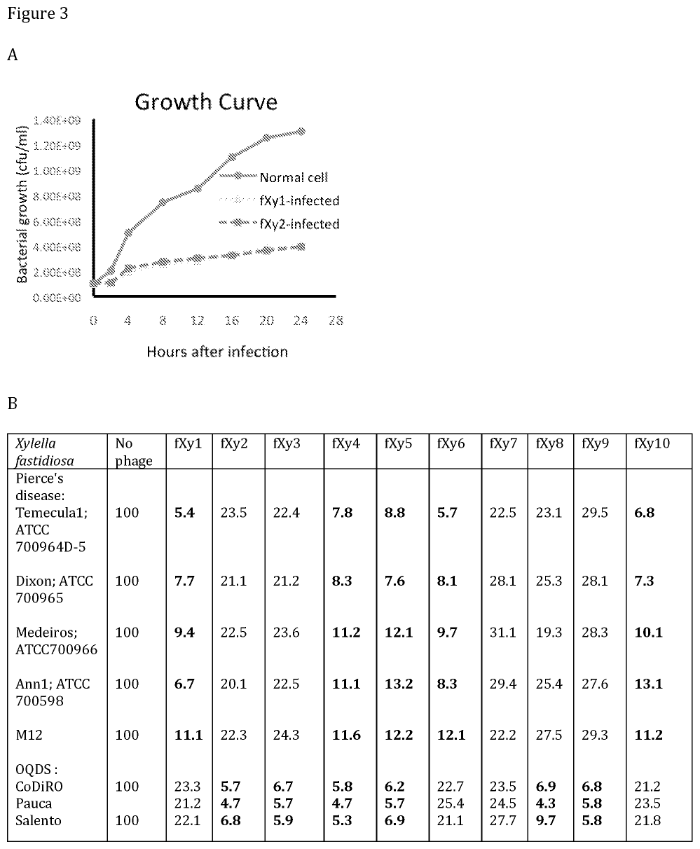

Results: Suppression of Xanthomonas and Xylella bacterial growth by phage infection. FIG. 3 shows that Xylella phages (SEQ ID NO: 1-10) have significant effect on Xylella bacteria growth. Overall more than 70% Xylella bacteria growth suppression can be found in all fXy phages. Specifically, 86.9-94.6% reduction of Xylella bacteria (pathogens for grape Pierce's diseases, ATCC 700964D-5 and ATCC700966; pathogen for almond leaf scorch disease, ATCC700965; pathogen for oleander leaf scorch, ATCC700598) growth was observed at 48 hours infection of fXy1, fXy4, fXy5, fXy6, and fXy10. Infection of fXy2, fXy3, fXy4, fXy5, fXy8, fXy9 phages significantly inhibited OQDS Xylella pathogens (CoDiRO, Pauca, and Salento) bacterial growth (>90%).

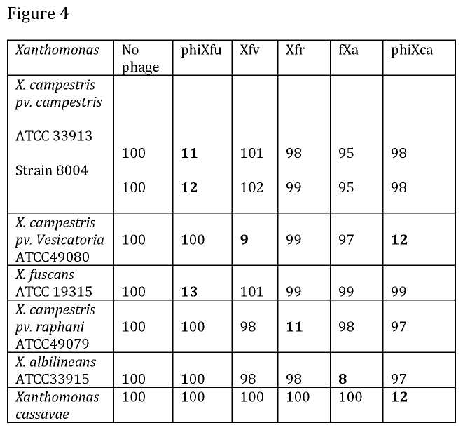

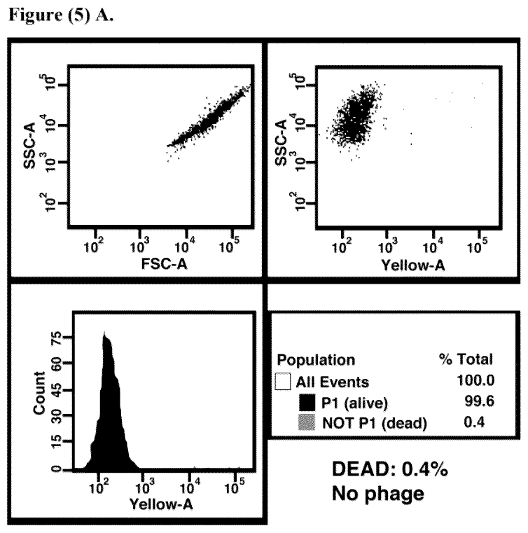

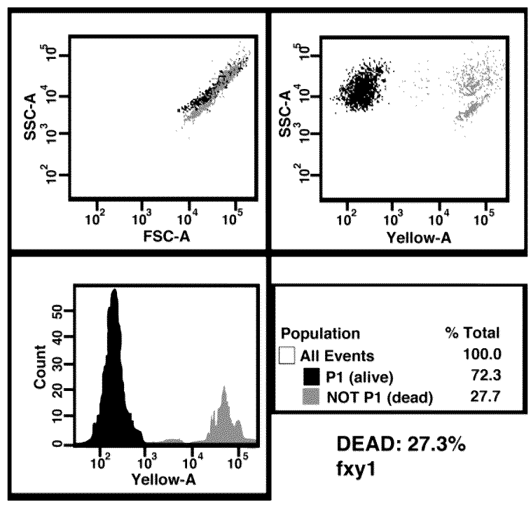

FIG. 4 shows the effect of Xanthomonas phages on different Xanthomonas pathogen growth. phiXfu phage (SEQ ID NO: 11) can suppress >87% growth of both Xcc (pathogen for black rot, ATCC33913 and strain 8004) and X. fuscans (pathogen for bacterial blight of bean, ATCC 19315). About 91% reduction of bacterial growth of X. campestris pv. vesicatoria (pathogen for bacterial leaf spot of tomato and pepper, ATCC49080) is found after Xfv phage (SEQ ID NO: 12) infection. Xfr (SEQ ID NO: 13) and fXa (SEQ ID NO: 14) phages drastically inhibit X. campestris pv. raphani (pathogen for leave spot disease, ATCC49079) and X. albilineans (pathogen for sugarcane leaf scald, ATCC33915) growth (89% and 92%, respecively). The growth of Xanthomonas cassavae (pathogen for cassava bacterial blight/necrosis) or Xcc is suppressed by phiXca phage (SEQ ID NO: 15) by 88%.

Taken together, Xanthomonas and Xylella phages can remarkably suppress the growth of pathogenic Xanthomonas and Xylella.

### Example 4. Cell Lysis and Death of Xylella and Xanthomonas Bacteria by Xylella and Xanthomonas Phages

The method to stain and quantify dead and live Xylella and Xanthomonas bacteria is performed as the following (Low et al., 2020), which is herein incorporated by reference in its entirety. Live/dead analysis was carried out for the quantification of total bacteria, as well as phage lysis of host vs. non-host bacteria. For quantification of phage lysis, overnight cultures of bacteria were diluted 1:50 into culture medium and incubated with 10⁸ PFU/mL Xylella and Xanthomonas phages. As negative control, no phage was added. Live/dead staining solution was prepared by diluting Syto 13 and propidium iodide (Thermo Fisher Scientific, Santa Clara, CA, U.S.A.) as 30 μM in Ringer's solution. After 24 hours infection, bacteria is suspended in the ratio of 1:50 into the live/dead staining solution and incubating for 25 min at room temperature in dark before acquisition on the FACSArray (BD). 20,000 bacterial cells are analyzed in each sample. For the quantification of phage lysis, a two-tailed Student's t-test was performed for each live/dead population to test for statistical significance between treatment with phage vs. no phage.

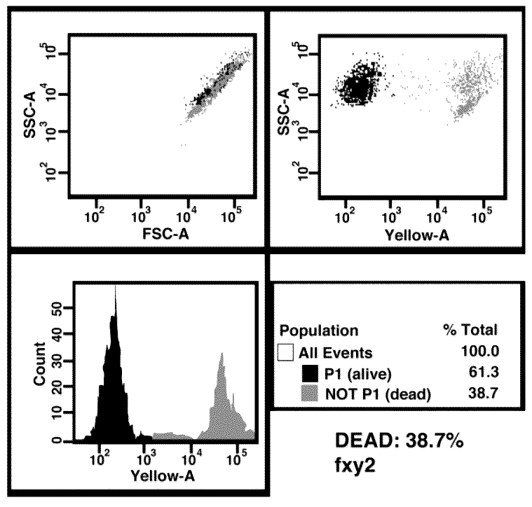

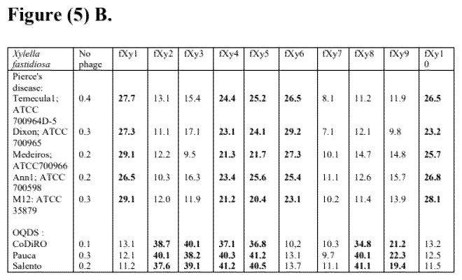

Results: Phage infection cause cell death of Xanthomonas and Xylella pathogens. X. fastidiosa and Xanthomonas bacteria were incubated with Xylella and Xanthomonas phages (SEQ ID NO: 1-15) in the culture medium for 24 hours. Bacterial viability was assessed using the red fluorescent propidium iodide (PI), because PI can only enter membrane-comprised (dead) bacteria. FIG. 5A shows examples of flow cytometric measurements of bacterial viability by FACSArray Bioanalyzer. There was only 0.4% bacteria stained by PI in control sample (no phage, left panel), indicating that membrane lysis and cell death are rare in the absent of phage. Infection with fxy1 and fxy2 caused very significant bacterial death rate of Temecula 1 (Pierce's disease) and CoDiRO (OQDS) (27.7% and 38%, respectively) within 24 hours (FIG. 5A, middle and right panel). FIG. 5B summarizes the cell death of various X. fastidiosa strains caused by Xylella phages. Generally, Xylella phages (SEQ ID NO: 1-10) leads 7.1% to 41.2% cell death, significantly more than the no phage control (p<0.001). fXy1, fXy4, fXy5, fXy6, and fXy10 led to robust cell death rate (more than 20%) of X. fastidiosa strains causing grape Pierce's diseases (ATCC 700964D-5 and ATCC700966), almond leaf scorch disease (ATCC700965), and oleander leaf scorch disease (ATCC700598). fXy2, fXy3, fXy4, fXy5, fXy8 and fXy9 phages killed 21.2-41.2% X. fastidiosa OQDS strains within 24 hours. These results indicate Xylella phages can cause bacterial membrane lysis and can be used to eradicate X. fastidiosa.

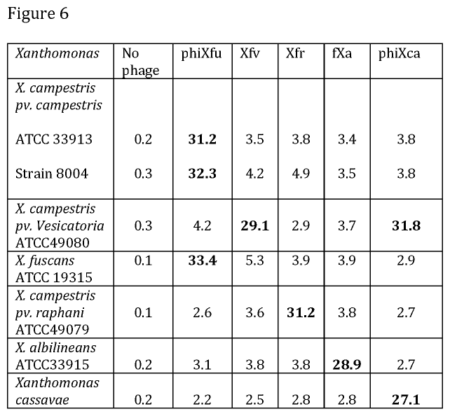

FIG. 6 shows the summary of the cell death rate of various Xanthomonas bacteria caused by Xanthomonas phages. phiXfu phage (SEQ ID NO: 11) killed of 31.2-32.3% Xcc and 33.4% X. fuscans. 29.1% cell death rate of X. campestris pv. vesicatoria is caused by Xfv phage (SEQ ID NO: 12) infection. Xfr (SEQ ID NO: 13) and fXa (SEQ ID NO: 14) phages eradicated 31.2% and 28.9% population of X. campestris pv. raphani and X. albilineans, respecively. phiXca phage (SEQ ID NO: 15) led to 27.1% and 31.8% cell death rate of Xanthomonas cassavae and Xcc, respectively. These results indicate Xanthomonas phages are lytic and able to eradicate Xanthomonas pathogens.

Taken together, recombinant Xanthomonas and Xylella phages can suppress growth and cause the death of Xanthomonas and Xylella bacteria.

1. An isolated or recombinant nucleic acid molecule comprising a nucleotide sequence, wherein said nucleotide sequence consists of SEQ ID NO: 1 to 10 or full length complements thereof; and (b) a nucleotide sequence having at least 80% sequence identity to SEQ ID NO: 1 to 10.

2. A vector comprising the isolated or recombinant nucleic acid of claim 1.

3. The vector of claim 2, wherein said the vector replicates in a Xylella host.

4. A host cell comprising the vector of claim 2, wherein said host cell is a bacterial cell and said bacterial cell is a Xylella fastidiosa, Xylella taiwanesis, Xylella sp. CFBP8070, Xylella sp. CL3.52, Xylella sp. PLS194, Xylella sp. PLS2, Xylella sp. PLS222, Xylella sp. PLS45, Xylella sp. WO2006108179 cell.

5. An antibacterial composition comprising at least one recombinant phage having lytic and inhibitory activity against a Xylella strain, said at least one phage being selected from the phages having a genome comprising a nucleotide sequence of anyone of SEQ ID NOs: 1 to 10 or a sequence having at least 80% identity thereto.

5(a). The composition of claim 5, comprising at least two, three, even more preferably at least four distinct phages selected from the phages having a genome comprising a nucleotide sequence of anyone of SEQ ID NOs: 1 to 10 or a sequence having at least 80% identity thereto.

5(b). A composition of anyone of claims 5 and 5(a), which inhibits bacterial growth and causes cell death of Xylella strains.

5(c). The composition of anyone of claims 5 to 5(b), which is a liquid, semi-liquid, solid or lyophilized formulation.

5(d). The composition of anyone of claims 5 to 5(c), which comprises between 10^{e4} and 10^{e12} PFU of each phage.

5(e). The composition of anyone of claims 5 to 5(d), for use for preventing the growth of Xylella microorganisms with a microbial growth inhibiting effective amount of a phage composition, which comprises recombinant Xylella phages (SEQ ID NO: 1 to 10) or their variants.

5(f). The composition of anyone of claims 5 to 5(e), for use for decontaminating a material Xylella bacteria.

6. An isolated or recombinant nucleic acid molecule comprising a nucleotide sequence, wherein said nucleotide sequence consists of SEQ ID NO: 11 to 15 or full length complements thereof; and a nucleotide sequence having at least 80% sequence identity to SEQ ID NO: 11 to 15.

7. A vector comprising the isolated or recombinant nucleic acid of claim 12.

8. The vector of claim 7, wherein said vector replicates in a Xanthomonas host.

9. A host cell comprising the vector of claim 8, wherein said host cell is a bacterial cell and said bacterial cell is a Xanthomonas campestris pv. campestris, Xanthomonas albilineans, Xanthomonas fuscans, Xanthomonas campestris pv. raphani, Xanthomonas campestris pv. vesicatoria, Xanthomonas cassavae, Xanthomonas arboricola sp., Xanthomonas axonopodis sp., Xanthomonas bromi, Xanthomonas campestris spp., Xanthomonas cassavae, Xanthomonas codiaei, Xanthomonas cucurbitae, Xanthomonas cynarae, Xanthomonas fragariae, Xanthomonas gardneri, Xanthomonas hortorum, Xanthomonas hyacinthi, Xanthomonas melonis, Xanthomonas oryzae sp., Xanthomonas pisi, Xanthomonas populi, Xanthomonas sacchari, Xanthomonas theicola, Xanthomonas translucens sp., X. vasicola, Xanthomonas citri, cell.

10. An antibacterial composition comprising at least one phage having lytic and inhibitory activity against a Xanthomonas species or strain, said at least one phage being selected from the phages having a genome comprising a nucleotide sequence of anyone of SEQ ID NOs: 11 to 15 or a sequence having at least 80% identity thereto.

10(a). The composition of claim 10, comprising at least two, three, even more preferably at least four phages selected from the phages having a genome comprising a nucleotide sequence of anyone of SEQ ID NOs: 11 to 15 or a sequence having at least 80% identity thereto.

10(b). A composition of anyone of claims 10 and 10(a), which inhibits bacterial growth and causes cell death of Xanthomonas bacteria.

10(c). The composition of anyone of claims 10 to 10(b), which is a liquid, semi-liquid, solid or lyophilized formulation.

10(d). The composition of anyone of claims 10 to 10(c), which comprises between 10e4 and 10e12 PFU of each bacteriophage.

10(e). The composition of anyone of claims 10 to 10(d), for use for preventing the growth of Xanthomonas microorganisms with a microbial growth inhibiting effective amount of a phage composition, which comprises recombinant Xanthomonas phages (SEQ ID NO: 11 to 15) or their variants.

10(f). The composition of anyone of claims 10 to 10(e), for use for decontaminating a material with Xanthomonas bacteria.

10(g). A host cell comprising the bacteriophage of claims 10 to 10(f), wherein said host cell is a bacterial cell and said bacterial cell is a Xanthomonas campestris pv. campestris, Xanthomonas albilineans, Xanthomonas fuscans, Xanthomonas campestris pv. raphani, Xanthomonas campestris pv. vesicatoria, Xanthomonas cassavae, Xanthomonas arboricola sp., Xanthomonas axonopodis sp., Xanthomonas bromi, Xanthomonas campestris spp., Xanthomonas cassavae, Xanthomonas codiaei, Xanthomonas cucurbitae, Xanthomonas cynarae, Xanthomonas fragariae, Xanthomonas gardneri, Xanthomonas hortorum, Xanthomonas hyacinthi, Xanthomonas melonis, Xanthomonas oryzae sp., Xanthomonas pisi, Xanthomonas populi, Xanthomonas sacchari, Xanthomonas theicola, Xanthomonas translucens sp., X. vasicola, Xanthomonas citri, cell.


SEQUENCE LISTING (15 sequences)

SEQ ID NO: 1 - DNA, 8233 bp, synthetic construct
  source 1..8233: mol_type = other DNA
tacgcgttatggctgttttcgttgctaaatttttcgctaatgctctgcgcctggctcgggttcgggcaggtaatgcccgcgccaatcagcagggtcgccgcggtcgcggtcaggcgtttatacagggttttccacgcttttttttcttcttcggtgcgcgcggtttcttcgcgaatcagcaccgcaatgctcggatcttctttcgccagtttaatcgccgccatcagatgttcatcggtaatgcggcgttcatctttgcgccactgcataatggtctggccgctcactttcagcgcttccgcaatcgccgcatcgcttttgcggctgcagctttctttcgctttatccagcagtttgttcagggtgctcatatcacctgcctgttgacaatcatcacctcctagtttacgttatctgatatcacctatagggtgataattagcccgagccatagcagctatccgggctggggcggcgtgctggatcgcgtgggccatggccgcagcgtggaaatgctgaaaattgtggaacattatccggcggaaggcatgagctttaaaagcctgggcgatcagagcgaaattagcgcgccgagcggcgtgccggtgaccgatcaggcggatagctgcgatctgagcccgcgcgtgggcccgctgctggcgggcattaaagaagtgatggatgatctgaacagcgcgctggataactttcatctgctggattttagcggctttgatacccataaagaagtgctgtttaaatggctggatattgtggtgggctttctggatgcgcgcgatgatctggaatttctggtgaacaaaattccgagcattgatattagcctggtgagcaaagatgtgtgatatgagcggcctgagcggctttgataacaacccgctgagcgcgcagcagtatattagcctgatgaccgcgagctttagcaccattgaaagcaacattgaaattattcagggcgtgctggtgcgcatgtatcgcattaccagcaaagtgagcaacgaatttatttttgataaaattcatctgatgtatattaacaccatggatattgtgagcgaactggaagaactgaaacatcgcctgagcagcctgagctatgcggattattgacactgagcggggtgcacgtatggcattaaatattgctgagatgtacgttcatcagttggaactttggccagatagtttgtctcagcagtttgatttttggatgtctgtccgtgctgcggcattagaacaactggatgagtgctgctgattaaagcgattattagcatgattaacgtgtttattctgtttctggtggtgattagcggcattctgaacctgtgcgcggcgcgctttctgacccgcgaacgccaggtgcatgtgtttgtgtttattagcagcttttatagcctgattgtggcggcggtgattctgtgctttgaaatttttaaaattctgtggtttcatcagtgatcaatttatggaattgcgagaagctgagttttgaatgtctctttttggttagggtggctattgttgcagttctttctattctttgtggggccttggcatgctttttcatctatctaattgtttggttgttgttttctccctttagctgggtattaattcccatatcgttcgcttttttttgtctattacgttattcaaagcattcataatcaaatatagttttcttgggcgaatttctgatggtgcattttattcagagcgtgtatagcaaatatgcgctgattcgcagctttttttattttccgtttgcgaccctgtttattaactttctgtttagctgctttctgctgatgctggcgtgcgtgtgcctggtgctgctggtgctggtgcagagcaccagcggctttcaggattattttctgcatagcgtgagccatagcctgagcagcccggtggcgagcctgtgcctgtatacctttgcgtttagcctgtgcaactgcgtgtttcgcacctgccatctgctgtgctggggcctgtggggccgccgcgatagcctggcgtttaaccgcagctaatttttatgtggcgctatctggtgagccgccgcagcagcagcagcgatagcgcggtgtatgtgaactttagcgaacgcgaacgccgcgcggatgtggaaaaagcggcgcgcctgtatgaaacccgccgcgatcgcgtggaaccgagctatgtgtttccggcgccgagcggcgaaaaaggcgtgggcccgaacagcaacaccggccagaaaggcgtggtgggcagctatccggtgagcattgattatctgaccgtggtgtttagctatgcgcgcctggcggaagcgggctattttgatgaaccgcgctttctgctgtatctgctgtttggcctgagcgtggatgatgtgattgtgggcagccataccagcgtgcgctggcatttttataacagcagcgcgagcattattgatagcaacggcgatctggtgggcaaaattggctgggatggcaacgcggatagctattgcattagcctgaccggcagcgcgtgccgctatattcatgattggagcaaagtgaaacgcagcctggcgagcctggatgcgcgcattacccgctgcgatgtggcgtatgatgattatgatggcattctgggcaccgtgcgccatcatgaagcgctggcgcgcgaacatctggcgccggcgggcggctgcctgctgtttagcagcggcggcaccccgccgcgcacccgctttctggatgatcatggcggcggcagcggctgcaccctgtatgtgggccagcgcggccataaacagctgtgcatttatgaaaaaggcaaacagctgggcgtggcggaaagcccgtgggtgcgctatgaagtgcgcctgtatgcgaaacatgcggtgattccgtttgatctgctggaagaaccgatgcgctatctgcgcggcagctatgattatctgtgccagctgtttagcagcgtggtggtgagcccggtgagccgcattcgcaccgtggtgaaacatgtggaagcgaccggcgaagcgctggtgcgctggctgcgccgccaggtgggcccggcgctgggcgtgctgcgccaggcgctgggctgcggctttagcgattttattgtggatcgcgtggaacgcgaaggcctgccgagccgctttcgccgcatttgccgcggcggcgatctgccggcgtatctgcgcgaaaccctggtggattgcagcgtgggcgtgtgcgtgtgagttagtgagggtttattttattttatttaatgattaggtgatttatgagcattgtgcgcgtgaaagataccgtgctgattgaacgcagcgtgaacaccaaaatgggcccgcaggtgtttcgcgaacagcgcgcgagcgtggtgatgggcggcgcgtatgaaaccgtgtttaacctgaaactgggcaccgatccgctgtattttaccggcgattatctgattcatccggatagctatggcaccgatgattatggcaacctggtgctgcgccgcattaaactgattagcctgagcagcgcgctgaaagaatttgcgagcaaagaaagcgtgagcctggtgagcagcaaagtggcgtaggagagttgaacatggcggtggcagtgttgcttgttcctgcatgtcgtgatgtggacatagatgcagtgagcggcaaatgtgctgccgtcgtgtgggtgcctcagccgtcgatgttccctgagttgagtattgccgatgcgcaatccatcggtgctgctatcttgttgttatgggctgtggcgtatgtgtttcgtgtgttacgtagactattttgaggttgaatatgttaaagcgtgtgctgagcgtggcgcgcccgagcgtgatttggggcggcctggtgagcctgctgtttagcccggcggtgtttgcggcggatgcgggcggcagcggcattgatgtgggcgcgattattgaagcgattaaagcggcgattggcccggtgggccagattggcattgcggtgctgagcgtgaccgtgctgctgcatgtgtataaatggattcgcaaagcgttttaaggcgtatttatttttttttgtttgtgttgaggtgggcggggaaggggtgttttgaataatgccccttcccagggagaagggggcgtatcgtgtttggttttatatttttgttcgcgttgtgtattgcgggcatgattattttttgggactagccgcgatgcgccgcgtgatttttctgttttttctggtgttttgcagcagcgtgtttgcggcgccgaaatgcagcgcgccgattgcgaccccggcgagcggcatttttagcgatcaggcggaagcgtatggcgcggcgcagagcaacctgcagtatcagctgtgcatgagcggctataccaacaccggccgcgtgaaaaccggcgatcgccagtatgtggtggtgctggatagcccgagcgcggtggtggattattatagctattataccagctgcagcgaaaaacagaacgatgtgtatacctttagcgataactattatagcaactttaacaacggcacccgcagctgctttgtgggctggaaaccgagcgcgaacagcagcagctgcgaagtggtgatgcatgcggatgcggcgacccatcgcattattgcggtgcataccggcaccgtgtgcgatgcgctgaactataaagatgattgcctgagccatccgggctatgtgtataaaagcggcggcagcggcgcggtgagcgcggtggaaggctatggcgattgcgtgccggcgaaaccgcagtgcagcctggatcagaaaaacgtgggcggcgtgtgcgtggataaatgcccggatggcatgctggaagatccggtgcgccatgaatgcgaatttgatcgcaaagaatgcccgctgggccagatgcgcgcgccggatggcacctgcgtgaaaaccagcgatagctgcccgagcggccaggcgcgcggcgcggatggcgcgtgcaaacgcgataaagatggcgatggcaaaccggatgatgaacagagcggcaaagatggcgatgaaggccgcagcagctttagctggagcggcggctgcgatgtggcgccgagctgcagcggcgatccggtgctgtgcggccaggtgcgcattcagtggcgcattgaatgcaacctgcgcccggatgtgaaagtgagcggcggcggctgcgatagcgtgccggtgtgcaccggcaaaaaatgcgatgcgatggaatatagcagcctggtgctgcagtggcgcaccgcgtgcgcggtggaacgcgcgaccggcgtggcgagcggcggcagcggcgatcgcgatccgaacgtggcggcgattaaagatgcgctgaccggcaaagatggcgtggtggataccggcgatgaaggcaaaccgagcagcgcgtttagcgatggcagcagcgcgggcggcgatgataaagatgcgagcaaatttgatgatcagggccgcggctatagccgcagctgctttgaaccgctgaccgtggatgtgtttggcagcgaactgaccattgatctgagcccgctgtgccagttttttcaggtgggcggccgcctggtgctgctgtttagcgcgctgagcagctttcgcattattagcggcattagcaaagaatagcggtaatgccgacctttctggcggcgctgctgaccggcctgtttaacctgtgcaaaagcaaactgggctattttgtggcgaccgcgtttgtgtggctgggcattaactggggcagctatcattttgtgattagcccgctggtggaacagctgtatacctatgtggatagcgtgggcaccgcgggcggccgctttggcgaactggcgctgcgcgcgctgggcctgctgaactttgatcgcgcgctgaccatgattattagcgcgtatgtgagccgctttgcgatgctgaacggccgcctgtatctgtttaaacgcggctatggcgtggcgccgccggcgagcccgaaacatgaaccgtttattccgccggcgggctaatgttatgcctattcatgttattactgcgcttcctggtggcggcaaaaccgcgattatggtggaaatgctggaagcggaagcgaaaattggcgcgcgcccgctgtttgcggcgggcattgatggcctgcagccgggcctggcgaccgtgctggatgatccgagccagtggaacgcgaaagatgcgcatggcaactatgtggtgccggatggcagcctggtgtttgtggatgaagcgtggaaatggtttggcatgaaatatggcggcagcggcatgcgccagaccaccccggatcatgtgcgcgcgctggcggaacatcgccatcgcggcctggattttgtgtggaccacccagcaggcgcataaacagctgtatagctttgtgcatggcctgattggccgccatacctttattaaacgccgctttggcacccgctttctggatgtgtgggaatgggatgaaatggtggaaaacgtgaacagcgcgagcaaccgcgaatttagccgccatcaggtgcgcaccctgccgaaacatgtgtatagcctgtataaaagcgcggaaattcataccattaaaagccgcattccgctgcgcctgctgctgattccgctgtgcgcggtgctgttttttgtgtgcgtgtggaacgcgtggcgcagcctgcgcccggatgcgattggcaccaccctgtttggccagcagagcccgcagaaagcggaaccggcgaaaccggtgggcaccgcgagccgcagcagcggcggcaccgtgggcggcggccaggcgccgcgctgggcgaccgcggtggcgtatgcgcgcgatcatctgccgcgctttccgaccatgccgtggaccgcgccgatttttgatagccgcagcctgaccgcggatccgcagctgatttgcatgagcggcggcgaaggcctggatgcgcagggccattataaaggcgcgagctgcacctgctataccgaacagggcaccctgtatgatctgctggaagcggaatgccgccgcattgcgcgcagcggcccggtgtataacccgtatcgcgaacgcgtgcaggatcgcgcggcggtgcagcagagcggcagcagcggcccggtgatgccggtgaacaccgaagtgcgcagcgtgagctttagcagcgcgggcagcctgccgtgagattttgctaccatagtttattgtcgatgtcaatagatgcatatatcttacatcttgtcaatagtttatgaattgttcataatctgaaaagtaattcatacttttgatggattgtcaataacgcaattatcatgccatttatgattcattcataattaatttattttatattcataattgtatgtgcagggtagggcagtctgattttttattgatgtatttctcagattaatgaaacagtttaataaacagggtcgccgcggtcgcgcccgccgccatcaggccgctcgcaatcaccaccggataccatttgctttctttgctcactttcagggtttcttcaatcagtttctgggtgcgcgcgttcatttcctgggtgcgcgcgttcatttcgttaatatgcgcgttcatttcctgggtatgggtggtcacttcatgaatttccgcctgaattttcgcggtttcgctaatcagtttggtaatctgaatgcgctgttcatcaataatttccgcgttatgttctttctgggtcatggtcatttttcctagtgttttctttacatagtgtagatcactgaggggtgtagggggctagccccctacgggctcgctttgcgtttctgctggcgctgaatcagcacctggcgcagcacaataatgctcgcggtgccgttcgcggtatggcggcgtttgcgcagcacggttttctggctcgcgttggtaatgcgctgcggatcgctcgccgcgcagccgccctgcggcgccgccgcgcgcgcttcttccatcattttctgccattcctgcgccaggctgcaggtcaggctcagccagcgcagctgccattcttcaatcgctttgcgttccggggtcaccagtttgccgttaataaacgcaaagccggtccagttgccggtcagtttctggttcggaatttcgttgctggtcactgcgcatgcgccagcggggtgccggtatggcgttttttcaggctaatcgccagttcgcgcacccagcggcgccagcggacataatatgcattatgtgaa

SEQ ID NO: 2 - DNA, 8538 bp, synthetic construct
  source 1..8538: mol_type = other DNA
ctctagcattgatttccaaatgcggcgttcggtttcggtttccgctttttcttcgcgcactttcgcaaacacggtttcatccgcgttcgcaatttcaatcagcagcgccagatgttcatcggtaatgcgcagttcatctttgcgccatttgctcatggtctggcggctcactttaatttttttcgccactttcgcatcgctgttcgcgctgcaggtttcgcgcgctttatccagcagtttgttaatggtgttcatgtcgcttgcttgttgacagatgtggtgcttgttagtttacacttcttcatgtcgcttgatgcgtgacatcacctccccattcatcctatcctggatggggaggtgctctaagacaggatagggcatggtaggagtgttgagatgctgcgcgtggcgacccgctatccggcggaaggcatgagctttaaaagcctgggcgatcagagcgaagtgagcagcgcgagcaaagtgtttgtgaccgatcaggaaggcgcgagcgatgcgagcagcgatattcgctttctggtggtgggcattaaaaaaattctgggcgatctgattattgcgcagcgcaactttgataccctgcgctttcgcgaatgcgatattggcatgtttcatcgcctgtgcattctgtgcgcgtgcagcaacctggaatatctgctggaaaacattagcgaatataaatgatctttttatttaaggtgctgtgatatgtctaaactatcagttttagatactgatcctcttttgctcatcagtatatttcttgtatgaatatatatgtttctgattctgaacctgcgcctgaaactgtttcgcggcgcgctggtgagcatgtttcgcgtggcgagcaaagcgagcgataacaaaattctggataaagtgcatctgatgtatatgagcagcctggatattgtgagcgaaattgaagaaattaaacagtatctgagcagcctgagcagcctgccgtttaaatgatatatgcgcattaccgataccgaacgcggcgcgcgcatggcgctgaacattgcggaaatgtatgtgcgccagatggatttttgggaagataccctgccgcagcagtttgatttttggctgagcgtgcgcgcggcggcgctggatcagctggatgaatgcgcgctgctgaaacagagcctgtattgatgatgaatttatggaattgcgaggagctaagttttgaatgtcttttttttgttaggttagctattgttgcagctctttctgttcttgttggggtcttgtcatgctttttcatttatctaattgtttggttcttgttttctgcctttttttgggtattaatacccatatcgttcgttttttttgtctatcacgttatgaaaaggatttgtaattattgaacttagtatgaggtttattcatgactcgaggtatttttgagtatttaacagactggtgtgtgagatgatgcgctttattcaggcggtgtatctgcgctgccatagcattcgcagctttttttattttccgattgtgcgccgcttttttaacctgctgctgagcttttttctgagcattctgctgtttatttgcctgagcctgctgattctggtgcagccgaccagcggctttcaggattattttacccatagcgtgagccatagcctgagcagctttgtgccgagcctgtgcagctatgtgtttgtgtttagcgtgttttattttattgtgcgcctggtgttttttctggattttctgctgctgctgtttctgaaccgcattaaccgctgcggctaatgctgagcgtggtgtttatgtggcgctatagcatgagccgccgcagcagcagcagcgatagcgcggtgtatgtgaactttctggaacgcgaacgccgcgcggatgtggaaaaagcggcgcgcctgtatgaaacccgccgcgatcgcgtgaacccgagctatgtgtttccggcgccgagcggcgaaaaaggcctgggcccgaacagcaacaccggccagaaaggcgtggcgagcagctatccggtgagcattgattatctgaccgtggtgtttagctatgcgcgcctggcggaagcgggctattttgatgaaccgcgctttctgctgtatctgctgtttggcctgaacccggatgatgtgattgtgggcagccataccagcgtgcgctggcatttttataacagcagcgcgagcattattgatagcaacggcgatctggtgggcaaaattggctgggatggcaaccgcgatagctattgcattagcctgaccggcagcgcgtgccgctatattcatgattggagcaaagtgaaacgcagcctggcgagcctggatgcgcgcattacccgctgcgatgtggcgtatgatgattatgatggcaccctgggcaccgtgcgccatcatgaagcgctggcgcgcgaacatctggcgccggcgggcggctgcctgctgtttagcagcggcggcaccccgccgcgcacccgctttctggatgatcatggcggcggcagcggctgcaccctgtatgtgggccagcgcggccataaacagctgtgcatttatgaaaaaggcaaacagctgggcgtggcggaaagcccgtgggtgcgctatgaagtgcgcctgtatgcgaaacatgcggtgattccgtttgatctgctggaagaaccgatgcgctatctgcgcggcagctatgattatctgtgcaaactgtttagcgtggtggtggcgagcccggtgagccgcattcgcaccgtggtgaaacatgtggaagcgaccggcgaagcgctggtgcgctggctgcgccgccaggtgggcccggcgctgggcgtgctgcgccaggcgctgggctgcggctttagcgattttattgtggatcgcgtggaacgcgaaggcctgccgagccgctttcgccgcatttgccgcggcggcatttatctgcgcatttgcgaacgccgctattaaattgttctgtgggcgtgtgtgtgtaagtcattgtggatttcttttaatttaattattaggtgatttatgagcattgtgcgcgtgaaagataacgtgctgattgaacgcagcgtgaacaccaaaatgggcccgcaggtgtttcgcgaacagcgcgcgagcgtggtgatgggcggcgcgtatgaaaccgtgtttaacctgaaactgggcaccgatccggtgtatccgaccggcgattatctgattcatccggatagctatggcaccgatgattatggcaacctggtgctgcgccgcctgaaactgatgagcctgagcagcgcgctgaaagaatttgcgagcaaagaaagcgtgagcgtggtgagcagcaaagtgagctaattagtgccgtgttatcgttttttgttttagttatttttttgtttttgtgttggtgtatagagttttttttgttgttttttgttgattatgtttttttttcttgtattgggaatgtttgttaagttatggcgctgtgcgtgagcctgaaagcggatggcaccctggtggtgaccggccagagcgtgagcgattgcaccggctatattctggtgagcggcagcgaatatggcgtgtatcagctggtgcagcgcgcgtttgaagtgccggatatgaaaaccgtgattcagaccagcaccgcggtggcgtttaccgtgattggctggtatgtggtggcgcgcattattggcaccgtggcgaccttttttgatggccgctaatcaataaacgaggtgatgtatggcggatattctgagcggcctggatgtgaaagcggcggcggcggcgctgattggcgcggcggcgctgattgcggtggtgggctttaccaaatggggcgcgaaaaaagtggcgggcttttttggctaaattattggagcggcacttttgtgtcgctccttcaccggctgggtgtatgtgctttgataattactttattctcttgttttttgggtgctttatgtggatgggctgctgttaaggggctggatgcttcatgagcatttttcgcattctggtgttttttgtgattctgtttattagccgcttttgctttgcgtgcgatattggcgaaccgcattgggatccgaaccagtgcctggatcgcggcgaagcgtatgcggtggcgagcgcgagctatcagatgtggcgcagcaacgaactgaaaaacagcaacattccgggcctgcaggtggtggattgcccgatgaccgatgatggccgcgtgattggctttggcggctatagcaccgcgccgggctatccgagcagcgatagctgcagcgatagccgcgtgtattttcagcgcgtgtatccggaaggcaaaacctgcctgacccgcagcagcaaaagcccggtggcgctgaccctgccgagcggcgtgcgcgtgagcagcaccgcgtgctatgatggctgcagctatgatgtggatcgcagcgatggcgtgattggcattggccaggatgatggccgcgtgcgctatgtgctgccgcgcatgaccccgaacggcaacctgtgcaccgtgagcccgagcggcgcgagcagcagcgcgagcagccaggataccccgccggcgcaggatgtggtgaaagatgaatgcacccgcatgggcaccctgacccagtgcgtgcgccaggatggcaaatattgcgcgaccagcagcaccggccatcagtattgctggaaaccgggcgaagtgggcacccagattgcgagcgatggcaaccatgcggcgaccctgaacaaaattgatgtgccggtgattgcgccggtggatgcgccgaaagataaaggcgattggcgcgtggatggcaaaggcaccagcacccagattaccaacaacacctataacaactataacaccaccacctttgcgagcaccggcagcagcggcggcagcagcggcggcagcagcggccagggcagcggcggcggcggcgataaaagcggcggcggcgataaaagcggcggcgataaaaacaccccgggcagcggcagcccgagcggcaccggcgtgctgtataaacgcaacggcaaaaccctggataccgtggtgagcggctatcaggcgaaagtgaacgatctgccgtttattagcggcattagcagctttctggcgattagcgcgagcggcgaatgcccggtgtttcgcctgagcgcgagcgcgtattggccggaaatgacctttgattatcattgcagcggcgtgtttctgagctttctgcgcagcgcgggctatattatttttgcgattgcgagctattttgcggtgcgcattgcgaccctgcgctaggtgggaggaatcatgtttattctgcgcgtgggctggctgaccgatctgacccagtggatttgggatctgattaccaaaatgtttctggcgctggcggattttgtgagcgatgtgtttgtgctgttttgcgatttttgctttagcctgattctgtttgtggtgggcgtgctgccgtggccggattttctgaaacagcagagcctgggcgatatgctgggcaaagcgggcagcaccgtggtgtggtttgcggatgtgtttcagctgagcaacagcatgcgcgtgattagcgcggcgattgtgtttagcatttttcgccgcctgctgaccctgggcatttggtgatgctggtgtttaacgaaggcgtgccgcgcagcggcaaaagctatgatgcggtgaaacatcatattctgccggcgctgcgcgaaggccgccgcgtgtatgcgcgcctgaacggcctgcgccatgaactgattgcgcagtatctggaaatgagcgaagcgcgcattcgcgaactgctgtttgtggtgaacaccgatgatgtgctgaacacctttgtgtgctatcgcgatgaagtggatggcaaatggtgcattgaagatcgctttaaagatgtgctgattgtgattgatgaagtgcatgaattttatgtggaaagccgcgcgccgctggcgccgcagattgaaaacttttgggcgctgctgggccagaacggcggcgatgcggtgctgatgacccagtggattaaacgcatgcatccggcgattcgcgcgcgcattgaacgcaaacatagctttcagaaactgaccgtggtgggcctgaaaaaccgctatcgcgtgacctattatcataccgtggcggcgggcaaatttgaaaaagtgggcagccagacctttaaatatgatgcgagcatttttccgctgtatgatggctatgcgccgggcgcgcgcaacaccgaagtgtatagccagggcaaacgcaccgtgtgggcggtgatgctgattaaagcgattttttttctggcggtgggcgtggtgggctttcatttttatagccgctattttggcggcgcgggcctgagcacccataacgtgagcagcccgagcagcagcggcgtgggccaggtgtttaaaccgggccaggtggtgagcggcccggtgcatcaggatgtgagcgcgccggtggtgccgccggtggatccgctgagcgatctgggcccggaacagcgctatatttttgatctgaacgcgaaaggccgcctgcgcctggcggcgctggcgcaggtgggccatgaatatcgcgcgtgggtgcagtggattaacaccgaaaacctggtgattgaacagctggatctggaacagctgcgcgcgctgggctttgatgtgagcgtgcatagctatggcgtgcgcattagcgtgctgagccatgtgctggtggcgaccgcgtggccgtggcgcgaaccggtgcgcgaaaccgatccgcgcctgtataacctgagccgcgatcagcagggcgcggtgagcattgcgagcgcggcgagcgatgcgcatggcgaaccggcggtgcagggcggcgtgattgaaaaaggcgaacgcgcgatgggcacctttccggaaagcccgggctatgaacatcatgatgaaagcggcggcggcagcctgtttagccgctagtttttgggtctttgtgaggaattttcattcacattttttattattttgtcaataatgcaattttcgtgccagttatgattggttcataatctatttatttattgttcataattgaatatgcagggtagggcggtctggttttttattgatgtggttctcagattagtggaatagtttgataaataaggttgttgctgtagctcctgctgccataaggctactcgctactactattggataccattttgattctgctgttattttttttttgtctgtgcatttacttcgtgaattagtgtaaatcaagcgggggtgtagggggctagccccctacggagacgcttctagtctgcgttctttgcagcgctgactagatcaaggttccgatttatcctccgcgttccctgcttacttcctcatgcaaaaccgctgttatggcgtgttggtgatgcgctaactctctttcgcggcaaagccgcccctgtaatttctccgccataccatccaaaaaaaaaaaagattaagaacctaggagagcttatgagctattttctgtgggatgatgcggcgctgcgctggctgagcgaaaaaagccgcaaagcgaccgcgcatgaagatgcggcgaaactgcgctggctggcgagctattttgcgggcaaaaaactggcggcgattgatagcgatctgattctgcgcgtggcgaccctgaaagcggcggaaaccagcccgagcaccgcgaaccgctatctggcgctgattcgcagcattctgcgccgcgcgtttgatatttggctgtggattgatcgctgcccgcatattagcctgtttccggaaccgaccaaacgcgtgcgctggctgaccagcgtgcaggcgcgcagcctgctgagcgaactgccgctgcatcagcgcgcgatggtgatttttgcgctgagcaccggcctgcgccaggcgaacgtgctgaaactgcgctgggatcaggtggatctggtgcgcaaagtgctgcgcattccggcggatcaggcgaaaggccgccaggcgattcgcattccgctgagcctgcatgcgctgcaggtgctgcaggcgcagcgcggccagcattatgaatgggtgtttacctattgcggccgcagcattcgctgggtgaacacccgcgcgtggcatcaggcgctgcagcgcgcgggcattcaggattttcgctggcatgatctgcgccatacctgggcgagctggcatgcgcagagcggcaccccgctgtatgtgctgcaggatctgggcggctggcagagcgaaagcatggtgcgccgctatgcgcatctgaccccgagccattatagcgcgtatgcggaagcggtgaccggctttctgccgtggtgctgcagccatccggcgggcttttaatgcgctgatcgctggtgcggttccagcacgggccgcccgcctggttcacgccttccagcgcttcgcggttcgcaatgctcgcatggctcgcgttcggcaggctcacgcccacgcccaccagcacggtcgccgcggtcgcggtcagtcgctctagcattgatttcca

SEQ ID NO: 3 - DNA, 9120 bp, synthetic construct
  source 1..9120: mol_type = other DNA
cttacgtaaggcatacccgcttactacggaatgcatcgtttgtcttctttctttgagttccaatgtgcgggcgttacgacggttgtaaactgcgtgcggccgaatttgtggatgctggtgtcgccgccgccactggctatgtacagtaaaaattatgttgctggttagtcttagctgtacactttacgatcgcaatctcatgtgctgcgttgttgttttgggccgttagctcaatcggtgctgcagcgcactttcaggccgctggtcgccggttcaatgcccgcgcggcccaccagtttgttcgcctgtttcagttcttcggtgttgcccaggcgcgcatagctggtgttcgccgcgctcacgcccacgcccaccaggcgttccagcatgcttttccacatggtgcgttcggtctggctggtcgcgcgttccgcatgaattttcgcaatccacgccgggccatccagtttcgcaatcgcgcacagctgcgcaatgcgttcatcgctcagcgggccgcgttcgttgcgcgcctgccacagcatctggcgggtaatgccaattttttcgcccagcgctttatcgctcggcacgcagcatttttcgcgcgctttatcaatcagttcgttaatcgcgttcatgtgaagttttttgttgactatgtgtagtgattactatactgtcttttttgtgaagtttttactacacatcccttcaatcttaccttgccgaggttggagggggtctggcaaggcactgtaggagtgttgagatgataaaggtagaaacgcgtaaaccagctgaagggatgagctttaaaagcctgggcgatcagggcgaactgagcgcggcgagcgaagtggcggtgattgatcagggccatagctgcattagcagccagcgcgtgggcgtgctgctggtgggcattgaagcggcgcgcagcgaactgctgagcgcgtttaaacgctatcagaaactgggcctggcgggctttggcattagcacctttgaagaacgccgcgaaattagcgattgcattcaggatgcgtataaccgcctggcgctgtttgtgaaacgcgcggaacgcattagccatgcgccgctgcgctatgaacagaccgaacgcgcgggctgcgtgagcgaactgtatcagccggtgagcgatctgccggatcagctgtattagcgatctgcagcattatattgatagcattgattggagcctgagcaaaatttttaccgatgcgagcgatgcgagcgatgaaattaccctggaaaccgtggaaagcatttttcagagcctgggcgaaattctgtttgaactgcgctttctggaagaacgcctgagccatctgagccgcgcgtagggtatgcgcattaccgataccgaacgcggcgcgcgcatggcgctgaacattgcggaaatgtatgtgcatcagctggatctgtggccggatagcctgccgcagcagtttgatttttggctgagcgtgcgcgcggcggcgctggatcagctggatgaatgctatctgctgcgccgcagcctgctgtgattgatgtgtttattctgtttctggtggtgaccgcgggcaccctgaacctgtatgcgacccgctttgtgacccgcgaacgcgaagtgcgcgtgtttgtgtttattagcgcgctgtttagcctgattctggcgagcgtgctgctgtgctttaaactgtttggcattagccgcttttaataatgattgatctgtttgatctgtggaacagcccgaaactgagctttgaatgcctgtttctggtgcgcctggcgattgcggtgtttattaccattctggcgggcggcctgagctgcttttttatgtttctgattgtgtggttttgctttagcgcgtttggctgggtgctgattctgatgctgttttttggctttattcatagcgcgatgcgctatattcatgatgaacgctatttttgagtttttattgtgaggcttgtgtgttttcttgatctgttattgctgtcgttcttaagtcgcagtcatcgctgcggttgatgctgtctctagtttttatgtggagttattctatgagccgccgcagcagcagctgcgatagcgcggtgcatgtggatttttttgaacgcgaacgccgcgcggcgctgaaaaaagcggcgtatctgtatgaaacccgccgcgatcgcgtgaacccgagctatgcgtttccggcggcgagcggcgaaaaagtgctgggcccgaacagcaacaccggccagaaaggcgtggtgggcagctatccggtgagcattgattatctgaccgtggtgtttagctatgcgcgcctggcggaagcgggctattttgatgaaccgcgctttctgctgtatctgctgtttggcctgagcgtggatgatgtgattgtgggcagccataccagcgtgcgctggcatttttataacagcagcgcgagcattattgatagcaacggcgatctggtgggcaaaattggctgggatggcaacgcggatagctattgcattagcctgaccggcagcgcgtgccgctatattcatgattggagcaaagtgaaacgcagcctggcgagcctggatgcgcgcattacccgctgcgatgtggcgtatgatgattatgatggcaaactgggcaccgtgcgccatcatgaagcgctggcgcgcgaacatctggcgccggcgggcggctgcctgctgtttagcagcggcggcaccccgccgcgcacccgctttctggatgatcatggcggcggcagcggctgcaccctgtatgtgggccagcgcggccataaacagctgtgcatttatgaaaaaggcaaacagctgggcgtggcggaaagcccgtgggtgcgctatgaagtgcgcctgtatgcgaaacatgcggtgattccgtttgatctgctggtggaaccgatgcgctatctgcgcggcagctatgattatctgtgccagctgtttagcagcgtggtggcgagcccggtgagccgcattcagaccgtggtgaaacatgtggaagcgaccggcgaagcgctggtgcgctggctgcgccgccaggtgggcccggcgctgggcgtgctgcgccaggcgctgggctgcggctttagcgattttattgtggatcgcgtggaacgccagggcctgccgagccgctttcgccgcatttgccgcggcggcgatctgccggcgtatctgcgcgaaaccctggcggattgcccggtgggcgtgtgcgtgtaggtcgttgtagatttctttatattttattgaatgattaggtgatttatgagcattgtgcgcgtgaaagataacattgtgattgaacgcagcgtgaacaccaaaaccgcgggcctgcagatttttcgcgaacagcgcgcggcggtggtgatgggcggcgcgtatgaaaccgtgtttagcctgaaactgggcagcggcagcgtgtatccgccgggcgaatatctgattcatccggatagctatggcaccgatgattatggcaacctgctgctgcgccgcctgaaactgattagcctgagcagcgcgctgaaagaatttgcgagcaaagaaccggtgagcgtggtgagcagcaaagtgacctgattagtgccgtgttatcgttttttgttttagttatttttttgtttttgtgttggtgtgtagagttttttttgttgttttttgttgattatgttttttttcttgttttgggaatgtttgttaagtcatggcgctgtgcgtgagcctgaaagcggatggcaccctggtgaccaccggccagggcgtgagcgattgcaccggctatgtgctggtgagcggcagcgaatatggcgtgtatcagagcgtgcagcgcgtgtttgaagtgccggatatgaaaaccgtgattcataccagcaccaccgtggcgtttaccgtgattggctggtatgtggcggcgcgcattattggcaccgtggcgaccttttttgatagccgctaatcaataaacgaggtgatgtatggcggatattctgagcggcctggatgtgaaaagcgcggcggcggcgctgattggcgcggcggcgctgattgcgctggtgggctttaccaaatggggcgcgaaaaaagtggcgggcttttttggctgaaatatctcgagcgacacttttagtgtcgctcttcaccggtgggtggggagggctgcactgtgatgattactttattctcttgttttcttggggctttgtgtggctgggccgctgttaaggggctggatgcttcatgagcatttttcgcattctggtgttttttgtgattctgtttattagccgctttagctttgcgtgcgaaattggcgaaccgcattgggatccgaaccagtgcctggatcgcggcgaagcgtatgcgattgcgagcgcgagctatcagctgtggcgcagcaacgaactgaaaaacagcaacattccgggcctgcaggtggtggattgcccgatgaccgataacggccatgtgattggctttggcggctatagcaccgcgccgggccatccgagcagccagagctgcgatagcagcctggtgtattttcagcgcgtgtatccggaaggcaaaacctgcctgacccgcagcccgaaaagcctgctgggcctgaccctgccgagcggcgtgcgcgtgccgagcaccgcgtgctatgatggctgcagctatgatctggatcgcagccatggcattattggcgtgggccaggatgatggcaaagtgcgctatgtgctgccgggcatgaccccgaacggcaacctgtgcagcgtgagcccgagcggcggcagcagcagcgaaagcagccaggaaccgccgccggtgcaggatgtggtgaaagatgaatgcacccgcatgggcaccctgacccagtgcgtgcgccaggatggcaaatattgcgcgaccagcagcaccggccatcagtattgctggaaaccgggcgaagtgggcacccagattgcgagcgatggcaaccatgcggcgaccctgaacaaaattgatgtgccggtgattgcgccggtggatgcgccgaaagataaaggcgattggcgcgtggatggcaaaggcaccagcacccagattattaacaacacctataacaactataacaccaccacctttgcgagcaccggcagcagcggcggcagcagcggcggcagcagcagcggcagcagcggcggcagcagcggcggcagcagcggcagcagcggcggcagcagcggcagcggcagcggcggcggcggcgataaaaccggcggcgataaagataccccgggcagcggcagcccgagcggcaccggcgatctgtataaacgcaacggcaaaaccctggatgcggtggtgagcggctatcaggcgaaagtgaacgatctgccgtttattagcggcattagcagctttctgaccattagcgcgagcggcgaatgcccggtgtttaccctgagcgcgagcgcgtattggccggaaatgacctttgattatcattgcagcggcgtgtttctgagctttctgcgcagcgcgggctatattatttttgcgattgcgagctattatgcggtgcgcattgcgaccctgcgctaggtgggaggaatcatgtttattctgcgcgtgggctggctgaccgatctgacccagtggatttgggatctgattaccaaaatgtttctggcgctggcggattttgtgagcgatgtgtttgtgagcttttgcgatttttgctttagcctgattctgtttgtggtgggcgtgctgccgtggccggattttctgaaacagcagagcctgggcgatatgctgggcaaagcgggcagcaccgtggtgtggtttgcggatgtgtttcagctgagcaacagcatgcgcgtgattagcgcggcgattgtgtttagcatttttcgccgcctgctgaccctgggcatttggtgatgctggtgtttaacgaaggcgtgccgcgcagcggcaaaagctatgatgcggtgaaacatcatattctgccggcgctgcgcgaaggccgccgcgtgtatgcgcgcctgaacggcctgcgccatgaactgattgcgcagtatctggaaatgagcgaagcgcgcattcgcgaactgctgtttgtggtgaacaccgatgatgtgctgaacacctttgtgtgctatcgcgatgaagtggatggcaaatggtgcattgaagatcgctttaaagatgtgctgattgtgattgatgaagtgcatgaattttatgtggatagccgcgcgccgctggcgccgcagattgaaaacttttgggcgctgctgggccagaacggcggcgatgcggtgctgatgacccagtggattaaacgcatgcatccggcgattcgcgcgcgcattgaacgcaaacatagctttcagaaactgaccgtggtgggcctgaaaaaccgctatcgcgtgacctattatcataccgtggcggcgggcaaatttgaaaaagtgggcagccagacctttaaatatgatgcgagcatttttccgctgtatgatggctatgcgccgggcgcgcgcaacaccgaagtgtatagccagggcaaacgcaccgtgtgggcggtgatgctgattaaagcgattttttttctggcgattggcgtggtgggctttcatttttatagccgcttttttcgcagcaccagcctgagcacccatgatgtgagcagcagcagcagcagcggcgtgggccaggtgtttaaaccgggccaggtggtgagcggcccggtgcatcaggatgtgagcgcgccggtggtgccgccggtggatccgctgagcgatctgggcccggaacagcgctatatttttgatctgaacgcgaaaggccgcctgcgcctggcggcgctggcgcaggtgggccatgaatatcgcgcgtgggtgcagtggattagcaccgaaaacctggtgattgaacagctggatctggaacagctgcgcgcgctgggctttgatgtgagcgtgcatagctatggcgtgcgcattagcgtgctgagccatgtgctggtggcgaccgcgtggccgtggcgcgaaccggtgcgcgaaaccgatccgcgcctgtataacctgagccgcgatcagcagggcgcgggcagcattgcgagcagcgcgagcgatgcgagcggcgcgccggcggtgcagggcggcgtgattgaaaaaggcgaacgcgtgatgggcacctttccggaaagcccgggctatgaacatcgcgatgaaagcggccgcggcagcagctttagccgctagttttttggtctctataaaagattttcattcatatttttattcttctgtcaataacgcaattatcatgccatttattattcattcatgatccatttattttatattcataattgtatatgcagggtagggcaggcttattttttattgatgtagttctcagattagtgaaatagtttgataaataaggttgctgctgtcgctcctgctgccatgagtccgctagccactactactggataccattttgattctgagtttagtttttttgattctgagtttagtttttctgattcattaattagtttttttgtgctggcgttaacttcatggatttcagcttgaatttttgctgtttctgctaatagttttgcaatttctgcatcatggttgaatgtgttcatgttcttttttttctttcttcagttgtgtcctttttatagtgtagatcaatagggggtgtagggggctagccccctacgggctcgctttgcgtttctgctggcgctgaatcagcacctggcgcagcacaataatgctcgcggtttcgttcgcggtatggcggcgtttgcgcagcacggttttctgcggcgcgttggtaatgcgctgcggatcgctcgccgccaggccgccctgcggcgccgcggtgcgcgcttcttccatcattttctgccattcctgcgccaggctgcaggtcaggctcagccagcgcagctgccattcttcaatcgcgcggcgttccggggtcaccagtttgccgttaataaacgcaaagccggtccagttgccggtcagaatctggttcggaatttcatcgttgctcattatgagcggactagttttttattgatacattgagattcttctatgttgcctgtaagtatttgatttgcatcattattgttactcattattcatgcaccagttttttgttggtcagcgccagcagtttgttaatcagttctttttccaggcgatacagcagttcgcgcacccagcggcggcaggtgctcataatcagcataatgcgaaacgggctcggcagaaaaaaaaagcaaaacgcgctgcgaaaaaaatcctgaatgcccgcgcgctgcagcgcctgatgccacgcgcgggtgttcacccagcgaatcgggcggccgcaataggtaaacacccattcatgatgatggccgcgctgcgcctgcagcacctgcagcgcatgaaagctcagcggcaggcgaatcgcctggcggcctttcgcctgatccgccggaatgcgcagcactttgcgcaccagatccacctgatcccagcgcagtttcagcacgttcgcctggcgcaggccggtgctcagcgcaaaaatcaccatcgcgcgctgatgcagcggcagttcgctcagcaggctgcgcgcctgcaccggggtcagccagcgcacgcgtttggtcggttccggaaacaggctaatatgcgggcagcgatcaatccacagccaaatatcaaacgcgcggcgcagaatgctgcgaatcagcgccagatagcggttcgcggtgctcgggctggtttccgccgctttcagggtcgccacgcgcagaatcagatcgctatcaatcgccgccagttttttgcccgcaaaatgaaacgccagccagcgcagtttcgccgcatcttcatgcgcggtcgctttatggcttttttcgctcagccagcgcagcgccgcatcatcccacagaaaatggctcataacctctcctaggttcttaatctttttgggatggtatggcggagaaattgcagggcggccttgccgcgaaagaggatcagcgcatcactaacgcgctataacagcggttttgcgtcgaatgaat

SEQ ID NO: 4 - DNA, 7810 bp, synthetic construct
  source 1..7810: mol_type = other DNA
tgcggttgatgctttctatagttttttatgtggaggtattttatgagccgccgcagcagcagcagcgatagcgcggtgtatgtgaactttctggaacgcgaacgccgcgcggatgtggaaaaagcggcgcgcctgtatgaaacccgccgcgatcgcgtggaaccgagctatgtgtttccggcgccgagcggcgaaaaacgcgtgggcccgaacagcaacaccggccagaaaggcgtggtgggcagctatccggtgagcattgattatctgaccgtggtgtttagctatgcgcgcctggcggaagcgggctattttgatgaaccgcgctttctgctgtatctgctgtttggcctgagcgtggatgatgtgattgtgggcagccataccagcgtgcgctggcatttttataacagcagcgcgagcattattgatagcaacggcgatctggtgggcaaaattggctgggatggcaacgcggatagctattgcattagcctgaccggcagcgcgtgccgctatattcatgattggagcaaagtgaaacgcagcctggcgagcctggatgcgcgcattacccgctgcgatgtggcgtatgatgattatgatggcattctgggcaccgtgcgccatcatgaagcgctggcgcgcgaacatctggcgccggcgggcggctgcctgctgtttagcagcggcggcaccccgccgcgcacccgctttctggatgatcatggcggcggcagcggctgcaccctgtatgtgggccagcgcggccataaacagctgtgcatttatgaaaaaggcaaacagctgggcgtggcggaaagcccgtgggtgcgctatgaagtgcgcctgtatgcgaaacatgcggtgattccgtttgatctgctggaagaaccgatgcgctatctgcgcggcagctatgattatctgtgccagctgtttagcagcgtggtggtgagcccggtgagccgcattcgcaccgtggtgaaacatgtggaagcgaccggcgaagcgctggtgcgctggctgcgccgccaggtgggcccggcgctgggcgtgctgcgccaggcgctgggctgcggctttagcgattttattgtggatcgcgtggaacgcgaaggcctgccgagccgctttcgccgcatttgccgcggcggcgatctgctggcgtatctgcgcgaaaccctgattgattgcagcgtgggcgtgtgcgtgtaagtcattatggatttctttaaatttaattattaggtgatttatgagcattgtgcgcgtgaaagataacgtgctgattgaacgcagcgtgaacaccaaaaccggcccgcagatttttcgcgaacagcgcgcggcggtggtgatgggcggcgcgtatgaaaccgtgtttagcctgaaactgggcagcgcgccgctgtatccgccgggcgaatatctgattcatccggatagctatggcattgatgattatggcaacctggtgctgcgccgcctgaaactgattagcctgagcagcgcgctgaaagaatttgcgagcaaagaaccgctgagcgtggtgagcagcaaagtgagctgattagtgtcgtgttatcgttttttgttttagttatttctttgtttttgtgttggtgtgtagagtttttttttgttgttttttgttgattatgttttttttcttgtcttgggaatgtttgttaagtcatggcgctgtgcgtgagcctgaaagcggatggcaccctggtggcgaccggccagggcgtgagcgattgcaccggctatgtgctggtgagcggcagcgaatatggcgtgtatcagctggtgcagcgcgtgtttgaagtgccggatattaaaaccgtgattcagaccagcaccaccgtggcgtttaccgtgattggctggtatgtggtggcgcgcattattggcaccgtggcgaccttttttgatagccgctaatcaataaacgaggtgatgtatggcggatattctgagcggcctggatgtgaaaagcgcggcggcggcgctgattggcgcggcggcgctgattgcgctggtgggctttaccaaatggggcgcgaaaaaagtggcgggcttttttggctaaattattggagcgacactttttgtgtcgctcttcaccggctggtgggggtgtgtgctttgatgattactttattctcttgtttcttgggtgctttatgcggatgggctgctgttaaggggctggatgcttcatgagcatttttcgcattctggtgttttttgtgattctgtttattagccgctttagctttgcgtgcgaaattggcgaaccgcattgggatccgggccagtgcctggatcgcggcgaagcgtatgcgattgcgagcgcgagctatcagatgtggcgcagcaaccagctgaaagatagcaacattccgggcctgcaggtggtggattgcccgatgaccgatgatggccatattattggctttggcggctataccaccgcgccgggctatccgagcagcgatagctgcagcaacagcctggtgtattttcagcgcgtgtatccggaaggcaaaacctgcctgacccgcagcccgaaaagcctgctgggcctgaccctgccgagcggcgtgcgcgtgccgagcaccgcgtgctatgatggctgcagctatgatctggatcgcagccatggcattattggcgtgggccaggatgatggcaaagtgcgctatgtgctgccgggcatgaccccgaacggcaacctgtgcgtgctgagcccgccgggcagcagcagcgaaagcagccaggataccccgccggtgcaggatgtggtgaaagatgaatgcacccatatgggcaccctgacccagtgcgtgcgcccggatggcaaatattgcgcgaccagcagcaccggccatcagtattgctggaaaccgggcgaagtgggcacccagattgcgagcgatggcaaccatgcggcgaccctgaacaaagtggatgtgccggtgattgcgccggtggatgcgccgaaagataaaggcgattggcgcgtggatggcaaaggcaccagcacccagattattaacaacacctataacaactataacaccaccacctttgcgagcaccggcagcagcggcggcggcagcagcggcggcagcagcagcagcggcggccagagcagcggcggcggcagcgataaaagcggcggcggcgataaaagcggcggcgataaagataccccgggcagcggcagcccgagcggcaccggcgtgctgtataaacgcaacggcaaaaccctggataccgtggtgagcggctatcaggcgaaagtgaacgatctgccgtttattagcggcattagcagctttctgaccattagcgcgagcggcgaatgcccgatttttaccctgagcgcgagcgcgtattggccggaaatgacctttgattatcattgcagcggcgtgtttctgagctttctgcgcagcgcgggctatattatttttgcgattgcgagctattttgcggtgcgcattgcgaccctgcgctaggttggaggaatcatgtttattctgcgcgtggcgtggctgaccgatctgacccagtggatttgggatctgattaccaaaatgtttctggcgctggcggattttgtgagcgatgtgtttgtgctgttttgcgatttttgctttagcctgattctgtttgtggtgggcgtgctgccgtggccggattttctgaaacagcagagcctgggcgatatgctgggcaaagcgggcagcaccgtggtgtggtttgcggatgtgtttcagctgagcaacagcatgcgcgtgattagcgcggcgattgtgtttagcatttttcgccgcctgctgaccctgggcatttggtgatgttggtctttaatgagggtgtgcctcgttctgggaaaagttacgatgcagtgaagcatcatattcttcctgctcttcgtgaggggcgtcgtgtttatgcacgtttgaatggtttgcgtgatgagcgaagcgcgcattcgcgaactgctgtttgtggtgaacaccgatgatgtgctgaacacctttgtgtgctatcgcgatgaagtggatggcgaatggtgcattgaagatagctttaaagatgtgctgattgtgattgatgaagtgcatgaattttatgtggaaagccgcgcgccgctggcgccgcagattgaaaacttttgggcgctgctgggccagaacggcggcgatgcggtgctgatgacccagtggattaaacgcatgcatccggcgattcgcgcgcgcattgaacgcaaacatagctttcagaaactgaccgtggtgggcctgaaaaaccgctatcgcgtgacctattatcataccgtggcggcgggcaaatttgaaaaagtgggcagccagacctttaaatatgatgcgagcatttttccgctgtatgatggctatgcgccgggcgcgcgcaacaccgaagtgtatagccagggcaaacgcaccgtgtgggcggtgatgctgattaaagcgattttttttctggcgattggcgtggtgggctttcatttttatagccgcttttttcgcagcaccagcctgagcgcgcataacgtgagcagcagcagcagcagcggcgtgggccaggtgtttaaaccgggccaggtggtgagcggcccggtgcatcaggatgtgagcgcgccggtggtgccgccggtggatccgctgagcgatctgggcccggaacagcgctatatttttgatctgaacgcgaaaggccgcctgcgcctggcggcgctggcgcaggtgggccatgaatatcgcgcgtgggtgcagtggattaacaccgaaaacctggtgattgaacagctggatctggaacagctgcgcgcgctgggctttgatgtgagcgtgcatagctatggcgtgcgcattagcgtgctgagccatgtgctggtggcgaccgcgtggccgtggcgcgaaccggtgcgcgaaaccgatccgcgcctgtataacctgagccgcgatcagcagggcgcgggcagcattgcgagcgcggcgagcgatgcgagcggcgcgccggcggtgcagggcagcatgattgaaaaaggcgaacgcgcgatgggcacctttccggaaagcccgggctatgaacatcgcgatgatctgggccgcgtgagcagctttagccgctagttttttggtctctataaaagattttcattcatatttttattcttctgtcaataacgcaattatcatgccagttatgattcattcataattcatttattttatattcataattgtatatgccgtgtagggcggcctagttttttattgatgtggttctcagattagtggaatagtttgataaataaagttgttgctgttttttatatctgtaggtttttcttgtaataaaagtatgcatatgctcatgattagcaatcttgtgtcttttatgagatgagcttatgaactttcgcattgtgagctttctgtttattttttttagcctgtttgtgagctgccgcgtgtttgcggatcagctggtgcataaatgcctgtataaacgccgcgtgagctatcagagcgaaccgtgcagcagcggcgtggaagtgcgcacctggcaggcggtgccggatagcccgccgagcagcgatgaactgctgcgccgctatcaggtggatcaggaactgcagcagcgctatgcgaacgatcgcctgcagcgcagcgtgagccataccctgggcagcagcattccggtgcagaaaagcagctgcgatgcggcgcgccgccagcgcgcggtgattctggatcagatgggcgtgaaccgcagctatgaagcgaccgcggcggtggataacgcggtgaacgcggcgtgccgctgaatacatagcttctacgctgtttattcaatgaaacagtttaataaacagggtcgccgcggtcgcgcccgccgcaatcaggctgctgcccaccaccaccggataccatttgctttcgccaatcagttttttggtctgcgcgttcacttcatgaatttccgcctgaattttcgcggtttccgcgttcagtttgctggtttcggtcagaattttcgcaatctgcgcttcgcgttcgttaataatttccgcgttatgttctttctgggtcatggtcattttctctcttgttttctgcctagtgtagatcaaacgggggtgtagggggctagccccctacggagacgcttctagtctgcgttctttgcagtgctgactagatcaaggttccgatttatcctccgcgttctccgcttacttcctcatgcaaaaccgctgttatggcgtgttagtgatgcgctagctctctttcgcggcaaggccgccctgcaatttcttcgccataccatcccaagaagattaataatctaggagagcttatggcgctgtgcgtgagcctgaaagcggatggcaccctggtggcgaccggccagggcgtgagcgattgcaccggctatgtgctggtgagcggcagcgaatatggcgtgtatcagctggtgcagcgcgtgtttgaagtgccggatattaaaaccgtgattcagaccagcaccaccgtggcgtttaccgtgatgagccattttctgtgggcggatgcggcgctgcgctggctgagcgaaaaaagccgcaaagcgaccgcgcatgaagatgcggcgaaactgcgctggctggcgagctattttgcgggcaaaaaactggcggcgattgatagcgatctgattctgcgcgtggcgaccctgaaagcggcggaaaccagcagcagcaccgcgaaccgctatctggcgctgattcgcagcattctgcgccgcgcgtttgatatttggctgtggattgatcgctgcccgcatattagcctgtttccggaaccgaccaaacgcgtgcgctggctgaccccggtgcaggcgcgcagcctgctgagcgaactgccgctgcatcagcgcgcgatggtgatttttgcgctgagcaccggcctgcgccaggcgaacgtgctgaaactgcgctgggatcaggtggatctggtgcgcaaagtgctgcgcattccggcggatcaggcgaaaggccgccaggcgattcgcattccgctgagcctgcatgcgctgcaggtgctgcaggcgcagcgcggccagcatcatgaatgggtgtttacctattgcggccgcagcattcgctgggtgaacacccgcgcgtggcatcaggcgctgcagcgcgcgggcattcaggattttcgctggcatgatctgcgccatacctgggcgagctggcatgcgcaggcgggcaccccgctgtatgtgctgcaggatctgggcggctggcagagcgaaagcatggtgcgccgctatgcgcatctgaccccgagccattatagcgcgtatgcggaagcggtgaccgaatttctgccgtagtgttgctaacgtccagcggacttttaatccgctggtcgctggttcgattccagcacggcccaccagcttgtttcagcccttctagtttttctctatttttcatgatgaattgagggtattttatttgtttttaatggcgaatggcgagatcaacatttttggggtccgattctcaaacatgtctcactgcgcttgcggttaagtcgtcaaaataaaaccacgcaatggttctattgttaatctgggcattgtcattaatagtgcatgtcattgttctgtcgctatccggtgtgaaaggcggaatattcactgcaatgcaatgctatggcgatgacaaattggcgttgcgcgattaggatgccgcattggaaggaggtaaggatca

SEQ ID NO: 5 - DNA, 9011 bp, synthetic construct
  source 1..9011: mol_type = other DNA
aatttttttgcccagggtttcgccgttcgccggaatgcacatgctaatcgctttatccagcagaatttttttcgctttcatatgaatcggcagctggcgcatgttaaattccagtttgcagcgcagctgcggggtaatgctccacatcacttcttcgttcatcctaccctggatggggaggtattctaggcagggtaggagtgttgagatgttgaaaacttttaagcgttatccagctgagggggtgtcttttaagtctttgggggatcaaagccaagtatctgcggcttctaaggtttctgttactgatcaggagcgggcttctaatgcgcctgcgcgtgctggtgtttctgattgcgggcattgaaaaagtgctgggcgatctgattattgcgcagcgcaactttgataccctgggctttcgcgaatgcgatattggcatgtttcatcgccgccgcgatattaccgtgtgcattctggatgcgtgcagcaacctggaatttctgctgaaaaacattcgcgaatataaatagtctgtttattcaaggcgctttcgcatgagcaacctgagcgtgttttatagcgatagcctgagcccgcagcagtatattagcctgatgaccgcgagctttagcaccattgaaagcaacattgaaattattcagggcgtgctggtgcgcatgtatcgcattaccagcaaagtgagcaacgaatttatttttgataaaattcatctgatgtatattaacaccatggatattgtgagcgaactggaagaactgaaacatcgcctgagcagcctgagctatgcggattattgacactgagcggggtgcacgtatggcattaaatattgctgagatgtacgttcatcagttggaactttggccagatagtttgtctcagcagtttgatttttggatgtctgtccgtgctgcggcattagaacaactggatgagtgctgctgattaaagcgattattagcatgattaacgtgtttattctgtttctggtggtgattagcggcattctgaacctgtgcgcggcgcgctttctgacccgcgaacgccaggtgcatgtgtttgtgtttattagcagcttttatagcctgattgtggcggcggtgattctgtgctttgaaatttttaaaattctgtggtttcatcagtgatcaatttatggaattgcgagaagctgagttttgaatgtctctttttggttagggtggctattgttgcagttctttctattctttgtggggccttggcatgctttttcatctatctaattgtttggttgttgttttctccctttagctgggtattaattcccatatcgttcgcttttttttgtctattacgttattcaaagcattcataatcaaatatagttttcttgggtgaatttttgatggtgcattttattcgcagcgtgtatagcaaatatgcgctgattcgcagctttttttattttccgtttgcgaccctgtttattaactttctgtttagctgctttctgctgatgctggcgtgcgtgtgcctggtgctgctggtgctggtgcagccgaccagcggctttcaggattatttttttgcgttttgcgtgagcttttttgtgtttacctgctgctttattgtgagcctgcatttttgcttttttagcctgcagctgtgcttttagaacctgtcatcttctctgttggggattatgggggcgtcgtgattctttggcctttaatcgttcttaatttttatgtggcgctgcctggtgagccgccgcagcagcagcagcgatagcgcggtgtatgtgaactttctggaacgcgaacgccgcgcggatgtggaaaaagcggcgcgcctgtatgaaacccgccgcgatcgcgtggaaccgagctatgtgtttccggcgccgagcggcgaaaaacgcgtgggcccgaacagcaacaccggccagaaaggcgtggtgggcagctatccggtgagcattgattatctgaccgtggtgtttagctatgcgcgcctggcggaagcgggctattttgatgaaccgcgctttctgctgtatctgctgtttggcctgagcgtggatgatgtgattgtgggcagccataccagcgtgcgctggcatttttataacagcagcgcgagcattattgatagcaacggcgatctggtgggcaaaattggctgggatggcaacgcggatagctattgcattagcctgaccggcagcgcgtgccgctatattcatgattggagcaaagtgaaacgcagcctggcgagcctggatgcgcgcattacccgctgcgatgtggcgtatgatgattatgatggcattctgggcaccgtgcgccatcatgaagcgctggcgcgcgaacatctggcgccggcgggcggctgcctgctgtttagcagcggcggcaccccgccgcgcacccgctttctggatgatcatggcggcggcagcggctgcaccctgtatgtgggccagcgcggccataaacagctgtgcatttatgaaaaaggcaaacagctgggcgtggcggaaagcccgtgggtgcgctatgaagtgcgcctgtatgcgaaacatgcggtgattccgtttgatctgctggaagaaccgatgcgctatctgcgcggcagctatgattatctgtgccagctgtttagcagcgtggtggtgagcccggtgagccgcattcgcaccgtggtgaaacatgtggaagcgaccggcgaagcgctggtgcgctggctgcgccgccaggtgggcccggcgctgggcgtgctgcgccaggcgctgggctgcggctttagcgattttattgtggatcgcgtggaacgcgaaggcctgccgagccgctttcgccgcatttgccgcggcggcgatctgctggcgtatctgcgcgaaaccctgattgattgcagcgtgggcgtgtgcgtgtaagtcattatggatttctttaaatttaattattaggtgatttatgagcattgtgcgcgtgaaagataacgtgctgattgaacgcagcgtgaacaccaaaaccggcccgcagatttttcgcgaacagcgcgcggcggtggtgatgggcggcgcgtatgaaaccgtgtttagcctgaaactgggcagcgcgccgctgtatccgccgggcgaatatctgattcatccggatagctatggcattgatgattatggcaacctggtgctgcgccgcctgaaactgattagcctgagcagcgcgctgaaagaatttgcgagcaaagaaccgctgagcgtggtgagcagcaaagtgagctgattagtgtcgtgttatcgttttttgttttagttatttctttgtttttgtgttggtgtgtagagtttttttttgttgttttttgttgattatgttttttttcttgtcttgggaatgtttgttaagtcatggcgctgtgcgtgagcctgaaagcggatggcaccctggtggcgaccggccagggcgtgagcgattgcaccggctatgtgctggtgagcggcagcgaatatggcgtgtatcagctggtgcagcgcgtgtttgaagtgccggatattaaaaccgtgattcagaccagcaccaccgtggcgtttaccgtgattggctggtatgtggtggcgcgcattattggcaccgtggcgaccttttttgatagccgctaatcaataaacgaggtgatgtatggcggatattctgagcggcctggatgtgaaaagcgcggcggcggcgctgattggcgcggcggcgctgattgcgctggtgggctttaccaaatggggcgcgaaaaaagtggcgggcttttttggctaaattattggagcgacactttttgtgtcgctcttcaccggctggtgggggtgtgtgctttgatgattactttattctcttgtttcttgggtgctttatgcggatgggctgctgttaaggggctggatgcttcatgagcatttttcgcattctggtgttttttgtgattctgtttattagccgctttagctttgcgtgcgaaattggcgaaccgcattgggatccgggccagtgcctggatcgcggcgaagcgtatgcgattgcgagcgcgagctatcagatgtggcgcagcaaccagctgaaagatagcaacattccgggcctgcaggtggtggattgcccgatgaccgatgatggccatattattggctttggcggctataccaccgcgccgggctatccgagcagcgatagctgcagcaacagcctggtgtattttcagcgcgtgtatccggaaggcaaaacctgcctgacccgcagcccgaaaagcctgctgggcctgaccctgccgagcggcgtgcgcgtgccgagcaccgcgtgctatgatggctgcagctatgatctggatcgcagccatggcattattggcgtgggccaggatgatggcaaagtgcgctatgtgctgccgggcatgaccccgaacggcaacctgtgcgtgctgagcccgccgggcagcagcagcgaaagcagccaggataccccgccggtgcaggatgtggtgaaagatgaatgcacccatatgggcaccctgacccagtgcgtgcgcccggatggcaaatattgcgcgaccagcagcaccggccatcagtattgctggaaaccgggcgaagtgggcacccagattgcgagcgatggcaaccatgcggcgaccctgaacaaagtggatgtgccggtgattgcgccggtggatgcgccgaaagataaaggcgattggcgcgtggatggcaaaggcaccagcacccagattattaacaacacctataacaactataacaccaccacctttgcgagcaccggcagcagcggcggcggcagcagcggcggcagcagcagcagcggcggccagagcagcggcggcggcagcgataaaagcggcggcggcgataaaagcggcggcgataaagataccccgggcagcggcagcccgagcggcaccggcgtgctgtataaacgcaacggcaaaaccctggataccgtggtgagcggctatcaggcgaaagtgaacgatctgccgtttattagcggcattagcagctttctgaccattagcgcgagcggcgaatgcccgatttttaccctgagcgcgagcgcgtattggccggaaatgacctttgattatcattgcagcggcgtgtttctgagctttctgcgcagcgcgggctatattatttttgcgattgcgagctattttgcggtgcgcattgcgaccctgcgctaggttggaggaatcatgtttattctgcgcgtggcgtggctgaccgatctgacccagtggatttgggatctgattaccaaaatgtttctggcgctggcggattttgtgagcgatgtgtttgtgctgttttgcgatttttgctttagcctgattctgtttgtggtgggcgtgctgccgtggccggattttctgaaacagcagagcctgggcgatatgctgggcaaagcgggcagcaccgtggtgtggtttgcggatgtgtttcagctgagcaacagcatgcgcgtgattagcgcggcgattgtgtttagcatttttcgccgcctgctgaccctgggcatttggtgatgttggtctttaatgagggtgtgcctcgttctgggaaaagttacgatgcagtgaagcatcatattcttcctgctcttcgtgaggggcgtcgtgtttatgcacgtttgaatggtgtgcgtgatgagcgaagcgcgcattcgcgaactgctgtttgtggtgaacaccgatgatgtgctgaacacctttgtgtgctatcgcgatgaagtggatggcgaatggtgcattgaagatagctttaaagatgtgctgattgtgattgatgaagtgcatgaattttatgtggaaagccgcgcgccgctggcgccgcagattgaaaacttttgggcgctgctgggccagaacggcggcgatgcggtgctgatgacccagtggattaaacgcatgcatccggcgattcgcgcgcgcattgaacgcaaacatagctttcagaaactgaccgtggtgggcctgaaaaaccgctatcgcgtgacctattatcataccgtggcggcgggcaaatttgaaaaagtgggcagccagacctttaaatatgatgcgagcatttttccgctgtatgatggctatgcgccgggcgcgcgcaacaccgaagtgtatagccagggcaaacgcaccgtgtgggcggtgatgctgattaaagcgattttttttctggcggtgggcgtggtgggctttcatttttatagccgcttttttcgcagcaccagcctgagcgcgcatgatgtgagcagcagcagcagcagcggcgtgggccaggtgtttaaaccgggccaggtggtgagcggcccggtgcatcaggatgtgagcgcgccggtggtgccgccggtggatccgctgagcgatctgggcccggaacagcgctatatttttgatctgaacgcgaaaggccgcctgcgcctggcggtgctggcgcaggtgggccatgaatatcgcgcgtgggtgcagtggattaacaccgaaaacctggtgattgaacagctggatctggaacagctgcgcgcgctgggctttgatgtgagcgtgcatagctatggcgtgcgcattagcgtgctgagccatgtgctggtggcgaccgcgtggccgtggcgcgaaccggtgcgcgaaaccgatccgcgcctgtataacctgagccgcgatcagcagggcgcgggcagcattgcgagcgcggcgagcgatgcgagcggcgcgccggcggtgcagggcagcatgattgaaaaaggcgaacgcgcgatgggcacctttccggaaagcccgggctatgaacatcgcgatgatctgggccgcgtgagcagctttagccgctagttttttggtccctataaaagattttcattcatatttttattcttctgtcaataacgcaactatcatgccatttatgattcattcataattcatttattttatattcataattgtatatgcaggggagggcagcctgattttttattgatgtagttctcagattaatgaaacagtttaataaacagggtcgccgcggtcgcgcccgccgcaatcaggctgctcgccaccaccaccgcataccatttgctttcggtggtcagtttcagggtttctttcatcattttctgggtatgcgcgttcacttcatgaatttccgcctgaattttcgcggtttcgctaatcagtttcgcaatctgaattttctgttcatcaataatttccgcgttatgttctttctgggtcattttcatcttttctagtgttttctttacatagtgtagatcactaagggggtgtagggggctagccccctacggagacgttttgtgcttttgttggcgttgtatcagcacttgccttaattggatgatcgacgcggtttcactggctgtgtgccttcttttacgcaaaaccgttttttttgaggtgcgttagtgatgcgctgcggatcggacgcggcacagccgccttgtggaactgctgctggcgctgccgggcgtgaccagctttccgctgatgaacgcgaacccggtgcagtttccggtgcgcacctggctgggcattagcagctttatgtggctgagcgtgtggctggtgtgctttctgagctgcctggtgagctttgtgagcctgctgctggtgcgcgtgagcgtgtttagcgtgtagcttttttccgtttagcctgaccagccataacgcgtattatgtgcgctggcgccgctgggtgcgcgaactggcgattagcctgaaaaaacgccataccggcaccccgctggcgcatgcgcatgaccagcaacgaaattccgaaccagaaactgaccggcaactggaccggctttgcgtttattaacggcaaactggtgaccccggaacgcaaagcgattgaagaatggcagctgcgctggctgagcctgacctgcagcctggcgcaggaatggcagaaaatgatggaagaagcgcgcgcggcggcgccgcagggcggctgcgcggcgagcgatccgcagcgcattaccaacgcgagccagaaaaccgtgctgcgcaaacgccgccataccgcgaacggcaccgcgagcattattgtgctgcgccaggtgctgattcagcgccagcagaaacgcaaagcgagcccgtagggggctagccccctacacccctcagtgatctacactatgtaaagaaaacactaggaaaaatgaccatgacccagaaagaacataacgcggaaattattgatgaacagcgcattcagattaccaaactgattagcgaaaccgcgaaaattcaggcggaaattcatgaagtgaccacccatacccaggaaatgaacgcgcatattaacgaaatgaacgcgcgcacccaggaaatgaacgcgcgcacccagaaactgattgaagaaaccctgaaagtgagcaaagaaagcaaatggtatccggtggtgattgcgagcggcctgatggcggcgggcgcgaccgcggcgaccctgtttattaaactgtttcattaatctgagaaatacatcaataaaaaatcagactgccctaccctgcacatacaattatgaatataaaataaattaattatgaatgaatcataaatggcatgataattgcgttattgacaatccatcaaaagtatgaattacttttcagattatgaacaattcataaactattgacaagatgtaagatatatgcatctattgacatcgacaataaactatggtagcaaaatctcacggcaggctgcccgcgctgctaaagctcacgctgcgcacttcggtgttcaccggcatcaccgggccgctgctgccgctctgctgcaccgccgcgcgatcctgcacgcgttcgcgatacgggttatacaccgggccgctgcgcgcaatgcggcggcattccgcttccagcagatcatacagggtgccctgttcggtatagcaggtgcagctcgcgccc

SEQ ID NO: 6 - DNA, 8980 bp, synthetic construct
  source 1..8980: mol_type = other DNA
cagcggcctttcaggccgctggtcgccggttcaatgcccgcgcggcccaccagtttaatcggttcgttcgcgctatcggtggtcgccactttcgcggtcgcgttaatcaggttcagaatgctctgccacacctggcgctgcgcgcgggtttccgcctgttcttccagcactttcaccacaatttcgccgttcagtttcgcaatcgccgccagttccgccgcatgtttttcggtaatcacgccgcctttgcgccatttgctcaccgcgctgcggctcacgccaatttttttgcccagggtttcgccgttcgccggaatgcacatgctaatcgctttatccagcagaatttttttcgctttcatatgaatcggcagctggcgcatgttaaattccagtttgcagcgcagctgcggggtaatgctccacatcacttcgccgttcatcctatcctggatggggaggtgttctagacagggtagggcacggtaggagtgttgagatgctgaacgaacagagcgaagtgagcgcggcgagcgaagtgagcgtgaccgatcaggcggatagctgcgtgcagagcaaaagcgtgtataaatttctgaccgcgctggaagtgctgaacgaaaccctgcgcgatgcgtttgattatcagagcaacctgagccagtattttaaagcgtttaacattcagatgtttgaagaacgcctggtgattctggatggcattagcgatgcgctgagcaccgtgcgcagcattattcgccatgtggaatgcgtgcgcgatgcgctggtgcgccatgtgtcgcctgagcggctttgataacgatagcctgagcgatcagcagtatattagcctgatgaccaccagctttagcaccctggaaagcaacattgaaattattcagggcgtgctggtgcgcatgtatcgcattaccccgaaaaccagctgcgaagtggtgtttgataaaattcagctgatgtatatttataccctggatattgtgagcgaactggaagaactgaaacgccgcctgagcagcctgagcgcgctgagcctgggctgatatatgcgcattaccgataccgaacgcggcgcgcgcatggcgctgaacattgcggaaatgtatgtgcgccagatggatttttggccggatagcctgccgcagcagtttgatttttggatgagcgtgcgcgcggcggcgctggatcagctggatgaatgctatctgctgcgccagagcctggtgtgattaattcaattattcttccttcagtaaattgcgtattaatcctcgtattaattttcgccattattaaatttgttgtgagttttattcatgatcgcaggtatttatgagtattttacaggcgggtgtgtcagatgattcgctttattcaggcggtgtatagccgctatcatagcattcgcagctttttttattttccgattgtgcgccgcttttttaacctgctgctgagcttttttctgagcattctgctgtttatttgcctgagcctgctgattctggtgcagccgaccagcggctttcaggattattttacccatagcgtgagccatagcctgagcagctttgtgccgtttctgtgcagctgcgtgtttgtgtttagcgtggtgtattttattgtgcgcctggtgttttttctggattttctgctgctgctgtttctgaaccgcattgaacgctgcggctgatgctttctatagttttttatgtggcgctattttatgagccgccgcagcagcagcagcgatagcgcggtgtatgtgaactttctggaacgcgaacgccgcgcggatgtggaaaaagcggcgcgcctgtatgaaacccgccgcgatcgcgtggaaccgagctatgtgtttagcgcgccgagcggcgaaaaaggcgtgggcccgaacagcaacaccggccagaaaggcgtggtgggcagctatccggtgagcattgattatctgaccgtggtgtttagctatgcgcgcctggcggaagcgggctattttgatgaaccgcgctttctgctgtatctgctgtttggcctgaacaccgatgatgtggtggtgggcagccataccagcgtgcgctggcatttttataacagcagcgcgagcattattgatagcaacggcgatctggtgggcaaaattggctgggatggcaacgcggatagctattgcattagcctgaccggcagcgcgtgccgctatattcatgattggagcaaagtgaaacgcagcctggcgagcctggatgcgcgcattacccgctgcgatgtggcgtatgatgattatgatggcgtgctgggcaccgtgcgccatcatgaagcgctggcgcgcgaacatctggcgccggcgggcggctgcctgctgtttagcagcggcggcaccccgccgcgcacccgctttctggatgatcatggcggcggcagcggctgcaccctgtatgtgggccagcgcggccataaacagctgtgcatttatgaaaaaggcaaacagctgggcgtggcggaaagcccgtgggtgcgctatgaagtgcgcctgtatgcgaaacatgcggtgattccgtttgatctgctggtggaaccgatgcgctatctgcgcggcagctatgattatctgtgccagctgtttagcagcgtggtggtgagcccggtgagccgcattcgcaccgtggtgaaacatgtggaagcgaccggcgaagcgctggtgcgctggctgcgccgccaggtgggcccggcgctgggcgtgctgcgccaggcgctgggctgcggctttagcgattttattgtggatcgcgtggaacgcgaaggcctgccgagccgctttcgccgcatttgccgcggcggcgatctgccggcgtatctgcgcgaaaccctggtggaatgcagcgtgggcgtgtgcgtgtaagtcattgtggatttcttttattttatttaatgattaggtgatttatgagcattgtgcgcgtgaaagataacattctgattgaacgcagcgtgaccaccaaaaccggcccgcagatttttcgcgaacagcgcgcgtgcgtggtgatgggcggcgcgtatgaaaccgtgtttaacctgaaactgggcaccgcgccggtgtatccgccgggcgattatctgattcatccggatagctatggcaccgatgattatgcgaacctgctgctgaaacgcctgaaactgattccgctgagcagcgcgctgaaagaatttgcgagcaaagaaccggtgagcgtggtgagcagcaaagtggcgtaggagagttgaacatggcggtggcagtgttgcttgttcctgcatgtcgtgatgtggatatagatgcagtgagcggtaaatgtgccgccgtggtgtgggtgcctcagccgtcgatgtttcctgagttgagtattgccgatgcgcaattaatcggtgctgctatcttgttgttatgggctgtggcgtatgtgtttcgtgtgttacgtaagctattttgcgcctgaacatgctgaaacgcgtgctgagcgtggcgcgcccgagcgtgatttggggcggcctggtgagcctgagctttagcccggcggtgtttgcggcggatgcggcgggcagcggcattgatgtgggcgaagtggtggcggcgattaaagcggcggcgggcccgattggcaccattggcattgcggtgctgggcgtgaccgtggcgctgcatgtgtataaatgggtgcgcaaagcgttttagggcgtatttactttttttgtttgtgttgaggtgggtggggaggggtgttttaaataatgccgctgccgcgcgaaaaaggcgcgtatcgcgtgtggttttatatttttgtgtgctttgtgtattgcggccattattatttttttggcctggcggcgatgcgcagcgtgatttttctgttttttctggtgttttgcagcagcgtgtttgcggaaccgaaatgcagcgcgccgattgcgaccccggcgagcggcgtgttttttgatcaggcggaagcgtatggcgcggcgcagagcaacctgcagtatcagctgtgcatgagcgattataccaacaccggccatgtgaaaaccggcgatcgccagtatattgcggtgctggatagcccgcatgcggtggtgggctattatagctatggcaccagctgcagcgaaaaacagaacgatgtgtatacctttagcgataactattatagcaactttaacaacggcacccgcagctgctttgtgggctggaaaccgagcgtgaacagcagcagctgcgaagtggtgatgcatgcggatccggtgacccatcgcattattgcggtgcataccggcaccgtgtgcgatgcgctgaactataaacaggattgcctgagccatccgggctatgtgtataaaagcggcggcagcggcgcggcgagcgcggtggaaggctatggcgattgcgtgccggcgaaaccgcagtgcagcctggatcagaaaagcgtgggcggcgtgtgcgtggataaatgcccggatggcatgctggaagatccggtgcgccatgaatgcgaatttgatcgcaaagattgcccgctgggccagatgcgcgcgccggatggcacctgcgtgaaaaccagcgatagctgcccgagcggccaggcgcgcggcgcggatggcgcgtgcaaacgcgataaagatggcgatggcaaaccggatgatgaacagccgggcaaagatggcgatgaaggcaaaagcagctttagctggagcggcggctgcgatgtggcgccgagctgcagcggcgatccggtgctgtgcggccaggtgcgcattcagtggcgcattgaatgcaacctgcgcccggatgcgaaagtgagcggcggcgcgtgcgatagcgtgccggtgtgcaccggcaaaaaatgcgatgcgatggaatatagcagcctggtgctgcagtggcgcaccgcgtgcgcggtggaacgcgcgaccggcgtggcgctgggcggcaacagcgatcgcgatccgaacgtggcggcgattaaagatgcgctgaccggcaacgatggcgtggtgaacaccggcgatgaaggcaaaccgagcagcgcgtttagcgatggcagcagcagcggcggcgatgataaagatgcgagcaaatttgatgatcagggccgcggctatagccgcagctgctttgaaccgctgaccgtggatgtgtttggcagcgaactgaccattgatctgagcccgctgtgccagtttctgcagattggcggccgcctggtgctgctgtttagcgcgctgagcagctttcgcattattagcggcattagcaaagaatagcggtaatgccgacctttctggcggcgctgctgaccggcctgtttaacctgtgcaaaagcaaactgggctattttgtggcgaccgcgtttgtgtggctgggcattaactggggcagctatcattttgtgattagcccgctggtggaacagctgtatacctatgtggatagcgtgggcagcgcgggcggccgctttggcgaactggcgctgcgcaccctgggcctgctgaactttgatcgcgcgctgaccatgattattagcgcgtatgtgagccgctttgcgatgctgaacggccgcctgtatctgtttaaacgcggctatggcgtggcgccgccgagcagcccgaaagtggaaccggtgattccgccggcgggctaatgttatgccgattcatgtgattaccgcgctgccgggcggcggcaaaaccgcgctgatggtggaaatgctgcaggcggaagcgaaactgggcgcgcgcccgctgtttgcggcgggcattgatggcctgcagccgggcctggcgattgtgctggatgatccgagccagtggaacgcgaaagatgcgcagggcaactatgtggtgccggatggcagcctgatttttgtggatgaagcgtggaaatggtttggcatgaaatatggcggcagcggcatgcataaaaccaccccggatcatgtgcgcgcgctggcggaacatcgccatcgcggcctggattttgtgtggaccacccagcaggcgcataaacagctgtatagctttgtgcatggcctgattggccgccatacctttattaaacgccgctttggcacccgctttctggatgtgtgggaatgggatgaaatggtggaaaacgtgaacagcgcgagcaaccgcgaatttagccgccatcaggtgcgcaccctgccgaaacatgtgtatagcctgtataaaagcgcggaaattcataccattcgcagccgcattccgctgcgcctgctgctgattccgctgtgcgcggtgctgtttattgtgtgcatgtggtatgcgtggcataccctgcgcccggatagcattggcagcaccctgtttggccagcagagcccgcagaaagcggaaccggcgaaaccggtgggcaccgcgagccgcagcagcggcggcccggtgggcggcggccaggcgccgcgctgggcgaccgcggtggcgtatgcgcgcgatcatctgccgcgctttccgaccatgccgtggaccgcgccgatttttgatggccgcagcctgaccgcggatccgcagctgatttgcatgagcggcggcgaaggcctggatgcgcagggccattataaaggcgcgagctgcacctgctataccgaacagggcaccctgtatgatctgctggaagcggaatgccgccgcattgcgcgcagcggcccggtgtataacccgtatcgcgaacgcgtgcaggaacgcgcggcggtgcagcagagcggcagcagcggcccggtgatgccggtgaacaccgaagtgcgcagcgtgagcgtgagcagcgcgggcgcgctgccgtgagttttgatagcatatatcatgctgatatttcaatggattcatacatttgtcttgttgtcaatagtttatgaattattcagaatggtagaagtcgctcatatttctgcattattgtgaataacgcaattatcatgccagttattattcattcatgatacattcaatttatattcataattttatatgtatgggaggggtattcgttttttttcttgtaataaacatatgtatgtgctcatgattagcaatcttgtatcttttataagatgagcttatgaacttttgcattaccagctttctgtttattttttttagcctgtttgtgagctgcccggtgtttgcggatcagctggtgcataaatgcctgtataaaaaacgcgtgagctatcagagcgaaccgtgcagcagcggcgcggaagtgcgcacctggcaggcggtgccggatccgccgccgagcagcgatgaactgtggcgccgctatcgcgtggatcaggaactgcagcagcgctatgcgagcgatcgcctgcagcgcagcgtgagccatagcctgggcagcagcattccggtgcagaacagcagctgcgatgcggcgcgcaaacgccgcgcggtgattctggatcagatgggcgtgaaccgcagctatgaagcgaccgcgagcgtggataacgcggtgaacgcggcgtgccgctgaatacatagcttttgcgctgtttatttaatgaaacagtttaataaacagggtcgccgcggtcgcgcccgccgcaatcaggctgctgcccaccaccaccggataccatttgctttccgcaatcagttttttggtctgcgcgttcacttcatgaatttccgcctgaattttcgcggtttccgcgttcagtttgctggtttcggtcagaattttcgcaatctgcgcttcgcgttcgttaataatttccgcgttctgggtcatggtcatttttcctgttgtttgctttgcatagtgtagagcagtaattgggggtgtagggggctagccccctacgggctcgctttgcgtttctgctggcgctgaatcagcacctggcgcagcacaataatgctcgcggtgccgctcgcggtatggcggcgtttgcgcagcacggtttttttcagatgggtcaggctcgcgctcacgcgtttgcgcggcagcgccgcgccgcaattcctccgccatatcattccaaaaagactaggaacctaggagaggttatgtctcattttttttctgtggggcgatgcggcgctgcgctggctgagcgaaaaaagccgcaaaaccaccgcgcatgaagatgtggcgaaactgcgctggctggcgagccattttgatggcaaaaaactggcggcgattgatagcgatctgattctgcgcgtggcgaccctgaaagcggcggaaaccagcgcgagcaccgcgaaccgctatctggcgctgattcgcagcattctgcgccgcgcgtttgatatttggctgtggattgatcgctgcccgcatattctgctgtttccggaaccgaccaaacgcgtgcgctggctgaccccggtgcaggcgcgcagcctgctgagcgaactgccgctgcatcagcgcgcgatggtgatttttgcgctgagcaccggcctgcgccaggcgaacgtgctgaaactgcgctgggatcaggtggatctggtgcgcaaagtgctgcgcattccggcggatcaggcgaaaggccgccaggcgattcgcattccgctgagcctgcatgcgctgcaggtgctgcaggcgcagcgcggccagcatcatgaatgggtgtttacctattgcggccgcagcattcgctgggtgaacacccgcgcgtggcatcgcgcgctgcagcgcgcgggcattcagaactttcgctggcatgatctgcgccatacctgggcgagctggcatgcgcaggcgggcaccccgctgtatgtgctgcaggatctgggcggctggcagagcgaaagcatggtgcgccgctatgcgcatctgaccccgagccattatagcggctatgcggaagcggtgaccgaatttctgccgtgatattgctaacgtccatcggccttttaatccgctggtcgctggttcgattccagcacggcccaccag

SEQ ID NO: 7 - DNA, 5626 bp, synthetic construct
  source 1..5626: mol_type = other DNA
atgagccgccagcagctgaccgaacatatttgccatctgcgcagcgcgggcagctttatttgcaaccatgatacctatgtgtgcctgagcgcgattgcgctgattgcggatgaactggatcatccgagcgtggcgagcctggcgtatagcattgtggatgatctgcatgattttagcgatctgctgacccgcctgagccatcgcctggaagaaagcctgagcgtggcggcggcgtagttttttatatgcgcattgatcatatgaaacgaagtaggagagtagggtatgcgcatcaccgatactgagcgaggtgctcgtatggcgttagagattgcagatattcaggcattgcgatcatcgcaatttgatttatttgaggtgattccagatcagttggagttttggtcccatgttcgtattgcggcattggatcagttggatgaatgtgctttattgaggcaagcattatgtctatacattatttgaacacctttcttttggccgttccctactttttttatatttttgcctgggctctatgctttcctttttaatcattgagtatttagacatctagcgatttacaattttctatgaactatctgatgacccgcctgctgaccccgtataaattttggtggcgccgcaccagccgcctggataaaattggcttttttgtggaagaaattctgccgcgctttgcgatttggtttaccctggcgctgtatgtgtgctttatttttatttaaatacatgtagtgggtgtttaaaagtgaaatgtcgatattctctcatcgcgtatcggtggtcatagtgtgtgcatcttgttgtcagtgcgtgtgcggcgttttgtgccgtgcaccactgtcaatgagatgtgcacacggtgccgtaggcaatgtccacggatacgtgagcccgtgatctttccgttgatgaggggtgatgagcctggcggaacgcgatgaacagctgtgccagctgctgaacacccagtttaaccagagcggcgtgcgcctgcagagcgcggcgtatgcggatctgctgcgccgcattccgtggcagcagttttggaccctgacctttcgcccgaccaacagcggctgcaacggcagcatgcatccggaagcggcggataaagcgtatcgctattttgtgagctgcattaaccgcagcctgtatggccgcgtgtggagcaaacgcccgcatcgcggcattcagtgggcgcgcggccaggaatggcatcgcgatggccgcctgcattttcatgcggtggtggcggcgccggatgaagatattaaccgcctgattaaccgctatgaatggcatgaattttggtatcgcgaatttggccgcaaccgcattgaagcgccgcgcagccagatggaaattaccggctatctgagcaaatatgtggcgaaaggcggcgaagtggatgtgagccgcaactttggcgcgtgggtgccgccgatgctggattataccccgcgcccggaacagtgcgcgctgattgcgggcgatcgcagcacctaattcggcgattggtccgtcatccaatcccccggtggcaggtggcaagccccgcactgtgggtactcaagcggttcttccttgtctcctgctggggggtaaggggggatcatagcttgaccccacagtaccgcccgaaatgatgcaacgaccaaagatgacgaagcgataaaagatgatgaaaagacgaaagatgagtaggcgacgcaagatgaaaagattacagatgaagatgaaacgagatgatgaatgaaccgagtgatcaagaggattaactgatggatatgccgaaagtgaccattatgagcggcgtgaacatgcgcgtggtgaccaccagcaaaggcgtgcagatgcagatttatagccagcgcgcggtgctggaaagcgaactgatgaaactgcagattgatgtggatgtggatggcctgaacgcgggctatccggtgggcgcgattaaatattgggatgtggtgaccgatctggtgccgggccagtatggcctgggcctggcgcgccgcatgaccctggtggatgcgaacgtgagcagcagcggccgcgcggtgccgccggtgagcgcgagcaaagtggcgtagtgatgccggtggcgaccctgctggtgagcgcgtgccgcgaagcggatgtggatgtggcgaccggccagtgcgcggcggtgatgtgggtgccgcagagcagcattctgccggaactgaccattgaaggcgcgcagctgatgggcgcgtgcattctgggcctgtgggcggtggcgtatgtgtttcgcctgctgcgcaaaagcattaaatgcgcgggcaaatgctgaaagcgatgctgcgcgtgctgcgccgcccggtggcgtggggcagcctggtgagctatgcgtttgcggcgagcgcgtttgcggcggatgcggcgggcggcggcggcattgatgtgaccgatgcggtgaacagcattaaacagggcaacgcgccgattgcggcgattggcattgcggcgctgggcgtggcggtgctgctgaaagtgtataaatgggtgcgccgcgcgctgtgagccgattgttattttgatgtgtttggggtgtcttggtcgggcaccccttttttcgaggtgggcatgatggcgggctggatttatttaggtgcgttatgtgtggcgggcctgattattttttggggcgatgattaatgcgcgtggtgctgtgcctgctgtgcctgatgtgcccgaccctggtgtttgcgggcccgaaatgcagcggccgcattctgctgagccagaactttcatgatcagggcgcggcgtatagcgcggcggtgagcagcctgaactatcagctgtgctttgatgatggccagagcagcggccatattgtgagcggcgtgaaacagtatattgcggtgctggatcatccgagcagcgtgattggctattatacctatgataccagctgcagcgatctgccgagcaaacgcattgaactgagcagcaccgcgattagccattttgtgaacggcacccgcagctgctttgatggctgggatattgatagcggcggcaccggcgcgtgcgaagtggtgaaacattttgatagcgcgaccaaaaccgtgattgaaacctataccggcagcgtgtgcgaagtgtttaactataaagatgattgcctgagccgcccgggctatacctatctggaaggcggcgcgggcagcggcgcgagcctggtgaacggctatggcgattgcgtgccgccgagcagcaaatgcccgagcgataaaaaactggtgaacggcgtgtgcgtgagcgattgcccggcgggcctggtggaagatcagaccagccatgaatgcgtggcgaaagatcagagctgcccgaccggccaggtgcgcgcgccggatggcagctgcgtgggcgataaagatccgtgcccgagcggccaggcgcgcggcgcggatggccagtgcaaaaccgatgcggatcatgatggcaccccggatgatgaacagagcaaagataaagatggcaaagataaaagcagctttagctggaaaggcagctgcgaagtggcgccgagctgcagcggcgatccggtgatgtgcggccaggtgcgcattcagtggcgcattgaatgcaacctgcgccgcgatgtgaaagtgaccggcggcgcgtgcgatgcgatgccggtgtgcaccggcgaaaaatgcaacgcgctggaatatgcgagcctgctgatgcagtggcgcaccgcgtgcgcgctggaaaaaagcaccggcgtggcgctgagcggcgataaagatggcgtgagcagcgatgtggcggtgattaaaaacgcgctgaccggcaaagaaggcgtggtggataccggcgatgaaggcaaagcgagcagcgtgtttagcgatgaaagcggctatggcaaagatggctatccgcagagcaaactggatgataaaggccgcggctatagccgcagctgcctggaaccgatgatggtggatgtgtttggcgaaaccattacctttgatctgaccccgctgtgccagtggctgcaggtgggcggcaaactggtgctgctgtttgcggcgctgagcagcgtgcgcattattgcgggcaccagccaggaataagcatgccgatttttattgcgaacgtgctgatgggcctgtttaacctgtttaaaaccaaactgggctatctgattgcggcggcgtgcgtgtggctgggcattaactggggcagctatcattttgtgattagcccgctggtggatcagctgtatacctatatggatagcgtgggcagcagcggcggccagtttggcgatctggcggtgcgcgcgctgggcattctgaacgtggatcgcgcgctgagcatgattattagcgcgtatgtggcgcgctttgcggtgctgaacggccgcctgtatctgtttaaacgcggctatggcggcaccccgccgaccagcgtgagctgacagggggcgtgtatgccgattcatgtgattaccgcgaccccgggcggcggcaaaaccgcgctgatgatggaaatgctgctggcggaaagcgaacgcgcgcagcgcccgatttttgcggcgggcattgatggcctggcgccgggcctggcggcggatctggcggatccgcgccagtggaacgcgaaaaacgatcagggcgaatatctggtgccgcatggcgcgattatttttattgatgaagcgtggaaatggtttggcagcgcgtttaacgcgagccatcagccgaccccgccgcatgcgctggaactggcggaacatcgccatcgcggcctggattttgtgtggaccacccagctggcgcataaacagctgtatccgtttgtgcgcggcctgattggccgccatacctttgtgaaacgccgctttggcaccagctggctggatgtgtatgattgggatcagctggtggaagaaaccgatagcagcaccaaccgccagtttagccgccatagcgtgcgccgcctgccgaaacatgtgtataaactgtataaaagcgcggaagtgcataccattaaaatgcgcctgccgtggcagctggtgctgattccgctgatggtggtggtgattgtggcgtgcgtgttttatgcgtatcgcagcctgattccgagcggctttagcagcagcattgaaggcacccgcagcaccccgggcaccggcggcgatgcgagcccgaaagcgggcgaagcgagccgcggcgaaggcccgcgctggccgaccgtgaccgcgtatgcgaaagatcatctgccgcgctttgcgagcatgccgtggaccgcgccgatttatgatcatcgcagcccgaccgtggatccgcagctgatttgcatgagcggcggcgcgggcctggatgcgcagggcgtgtataaaggcagcagctgcacctgctataccgaacagggcaccgtgtatgatattagccagctggaatgccgcaccattgcgcgccgcggcccggtgtataacccgtatcgcgaacgcgtgcaggaacatagcgcgatgcagcagggccaggtgagcagcagcctggtgcagggcagcaccgcgaaacaggtgagcgtgagcgcggcgggcgcgctgccg

SEQ ID NO: 8 - DNA, 6800 bp, synthetic construct
  source 1..6800: mol_type = other DNA
agttgatgtcataacaagtttcccttagttacctgcatggttacatgatgccatgcataaaattgatgtgtgtggggggggtagggggtgtgccccctacggtgacgctctaaactcgtgttattgcagcgctgactagattaaggtttcgatattttctccgcgtccccagctcactatcttccgtaaaccgcttttaggacggcattgcaatgtctaggtgtgattagtggcattttgccagcctagttattttctcctttattttcatgatgttactgagttttttccatagtgtttttattctttgccgctgccgcgcactttcaggccgctggtcgccggttcaatgcccgcgcggcccaccagtttgttcggctgtttcagttcttctttgttgcccatgctcgcatggctggtgttcgcggtgctcacgcccacgcccacaatcagcgccgccgcggtcgcggtcaggcgtttatacagtttttcccattctttgctcagttcgccctgatcctgttcgctgcgaatcagcagcagccactggcccgcatcttcgcccgcaattttcgccagttcctgaatgcgttcgttcggaatcggtttgttgccgtttttccaatcatacagcacgccatgcgcaatatcaatgcgttccgccagcgcgcgcacgtttttgcgatccaggcgctgcaccgccagttcaatcagtttggtcgcatgcatattttttgtctctaaatgtagacatttgactctattaagagtacatttttatttgtctctaatcagggacaatcatctcccccttcatcctatcctggatggggaggtgttctaagacaggataggacacggtaggagtgttgaggtgatgaaagtggaagaacgcaacccgcatgaaggcatgagctttaaaagcctgggcgaaccgagcgcggtgagcaaacgcctgctgctggcgagcattgcgaaactgattgaagatctggatagcgtgaaaagcaaatttgaagatctggattatgaagaatgcggcattgcgatgcataaagaacgccgctatgtggcgtattgcgtggatgaagcggcggaatatatgagccgctttctgggcgatattgatgatgattggctggtgatttagaggacgatttaaatgagcaaactgagcgtgctggatgcggatctgagctttaccagcgaatatctgaacagcctgaccctgtttattagcgatctgaaacattataccgatagcctggatggcagcctgagcaaaatttttaccgatgcgagcgatagcagcgatgaaattaccctggaaattattcagagcatttatctgagcctggcggatattatttatgaactgtttagcctggaagcgcgcctgagctgcctgagcagcagccatctgcgctgatatatgcgcattaccgataccgaacgcggcgcgcgcatggcgctgaacattgcggaaatgtatgtgcgccagctggatttttgggaagataccctgccgcagcagtttgatttttggctgagcgtgcgcgcggcggcgctggatcagctggatgaatgccatctgctgcgccagagcctgctgtgaacgattcaatcatcctttccttggtaattattgccggttttcttaattttctttctgtatgtctttgggggaagaatcgtaaatctcatgtttttattttcttttctacactctattctcttgttttagctactgctcttcttgttcttaggttttattgatgctgaacctgtggaacagcgaaaaactgagctttgaatgcctgtttctggtgcgcctggcgattgtgaccgtggtgtttattctggcggtgggctttagcttttttagcatgtttctgattgtgtggctgtttagcagcggctttggctgggtgctgattccgattagcaccgtgacctttgtgtattatgtgatgggcaaaatttttaaccagatttagttgcttttagttattgaaaacattgtttggagaatatcaatgaatgacttttattctttcatcgcgtatcggtgtgcgtaatgtgtgcatcttgttggcagtgcgtgggcggtgtttctccgtccatccactgtcaataagatgtgcatatggtgcgaagcaacgtgcacggataggcgatctagtcatgtttaagtgggagaaatgagcctggaagaaagcgatgcgcagctgtgcaaactgctgaacattcagtttaaccagagcgcggcgcgcctgcagagcgaagcgtatgcggatctgctgcgccgcagcagctggcagcagttttggaccctgacctttaaaccgaaaaacagcggcagcaacggcagcatgcatccggaagcggcggataaagcgtttcgcttttttgtgagcaacattaaccgcgcgctgtatggccgcatgtggagccagcgcccgcatcgcggcattcagtgggcgcgcggccaggaatggcatcgcgatggccgcctgcattttcatgcggtggtggcggcgccggatgaagatattaaccgcctgatgagccgctatgaatggcatgaattttggtataaacgctttggccgcaaccgcattgaagcgccgcgcagccagatggatattaccggctatgtgagcaaatatgtgagcaaaggcggcgaagtggatgtgagcccgaactttggcgcgtgggtgccgccgaaaattgattatagcccgcgcccggaacagggcaccctgattaccggcgatagcggcaaataatccggcgattggtccgttatccagtcccctggtggcaggtggcaagccccgcactgtgggttcaaagcaagttcttccttgtctctcgcatggggggtaagggggggtcttagcttgaccccacagtagcgaccaaaattgatgaaacgacgaaaaaagatgagatgacgaaagacgaaagacgaaagacgaaagacgaaagacgaaagacgaaagacgaaagacgaaagacgaaagacgacgatattaactgaaatgaattaatcaatgaatgagggtgaatcaatgcaagttcaaatgccaaaagtaacgattaagtctgatgtagctatgcggactgtgactacgcaacaaagcgcataaatgccgcgtggatggcctgcagcagggctatagcgtgggcgcggtgaaattttgggatgtggtgagcgatctggtgccgggccgctttggcattgatctgagccgcaaaaaaaccctggtggatgcgaacgtgagcggcagcggccgcagcctgccgagcccggcgccggcgagcagcagcaaaggcagctaaacatgacggcggcaacgttactcgttcctgcctgccgtgaggcggatatagatgcagtgagcggcaaatgtgctgccgtgatgtgggtgccgcagtcgtccatgcttcctgagttgagtattgccgatgcgcaatcaatcggttctgccatcttgttgttatgggctgtggcttatgtgttccgtgtgttacgtaaattattgatatgcgcagcaaaatgtttaaacgcattgtgattgcgagcaaaagcaaactgccgattgtgtttggcgtgatgctgagctatgcggtggtgagcccggtgtttgcggcgggctttgatgtgagcaaagtgattgaagcgattcaggaagcgatgaccccgattagcagcattggcggcaccgtgctgctgattattgcgctgctggcgacctataaatgggtgcgcaaagcgttttaaggatataggttattccttcgatgtctcggggtgtctgtgcatggccatccgctgattagccgctggcatgatggccgcgtggatatttttcgcagcgtgatgtgcggcggcagctattattttctgggcctgggcctgatgcgcggctttattctgctggcgctgctggtgtttccgctgtatgcgtggggcaaaagctgcggcgatggcggcccggtgggcggcggccattatccgcatcagggccaggcgtatgaagcggcggtgaaaatggcgaactttcaggattgctggatgggccgcaacgaaagcaaagtggaacatcatccgcgcgatcgcctgtatgcggtgcgcaccccgggcgcgattgcgggctattattattatgatgatacctgcgaaagccgcagcagccgcattaccccgcgcaacccggtgaccggcagcctgggctgcaacagcggctgcctggtgagctttattcgcaacggcgatgatgaaaccgcgaccgaaagcaccaccggcgaagtgtgcgatggcgataaactgcgcgaaatttgcgaaagcaaaagcggcaacggcgcgaactatgtgtatgtgccgtttgtgcgcgtgtgccagccggtgcagccggaatgcccggaaggccagcagccgaaacatggcgtgtgcgaacgcctggatgcgtgcccgcatggcaccattaccgataaacagacccaggaatgcattccggaaaaagaagcgtgcccggtgggccaggtgcgcgcgccggatggcagctgcaccggcgaaaaagaaaaaaacgaatgcccgagcggccaggcgcgcggcgcggatggcacctgcaaagcggatgcggatcatgatggcattccggatgatgaacaggatagcaaagaaaaagataaagataaagataaagataaagataaaggcagcagctttagcggcggcgaacgctgcgatgtgccgccgcagtgcagcggcgatccggtgatgtgcggccaggcgcgcattcagtggcgcattgaatgcagcctgcgccgcgatgtgaaaattaaaggcggcacctgcgatgcgcagccgatttgcaccggcgaaaaatgcaacgcgatggaatatgcgagcctgctgctgcagtggcgcaccgcgtgcgcgctggaaaaaggcagcgcgggcgcggtgctgggcggcgataaaaaccagggcaacgcggatgtgggcgcgattaaagatgcgctgaccggcaaagatggcaccgtggataccggcgaagaaggcgatccgagcggcgcgtttagcgatgaaagcggctatggccgcgaaggctatccgaaagtgaaaattgatgataaaggccgcggctatagccgcgtgtgcccgaaaagcattagcattaacgtgtttggcaaacagattattattagcctgaccccgatttgccagtttctgcagattggcggcaaactggtgctgattctggcggcgctggcgtgcctgcgcattatgggcggcagcctggaaggctgatctatgccggtgtttattgcgcagctgctgaccggcctgtttagcctgctgaaaaccaaactgggctattttattatggcggcgtttgtgtggctgggcattaactggggcagctatcattttgtgattaccccgattgtgaaacagatttatatttatatggataacgtgggcgtggcggaaggcagcctgggcgaaatggcgatgagcggcctggcgattctgaactttgatcgcgcggtgagcatgattattagcgcgtatgtggcgcgctttgcggtgctgaacggccgcctgtatctgtttaaacgcggctttggcggcaccccgggcgcggcgccgtaatgccgattaccgtgctgaccggcaccccgggcggcggcaaaaccgcgctgatggtggaacgcctgctggaagaaagcaaacgcgcggaacgcccgctgtttgcggcgggcattgatggcctgcagccgccgggcctggcgaccgtgctggaagatccgcgccagtggaacgcgaaagatgcggaaggcaactatctggtgccgaacggcgcgatgatttttattgatgaagcgtggaaatggtttggccatctgcatgatgcgagccgccagccgaccccgcattatgtgctggaactggcggaacatcgccatcgcggcctggattttgtgtggaccacccagcaggcgcagcagctgtatccgtttgtgcgcggcctgattggccagcatgtgtttctgaaatgcatttgattccgctgctgattgtgctgattgtggtgtgcattgtgattaccgtgcgcagcctgcgcagcagcagcagcaacatgctgcgcacccaggaaggcgcgaacggcgcggaactggcgaaaccgggcgcgccggcgagccatcatccggtgggcggcgaacatggcgatgcgctgcgctgggaaaccgaaatggcgtatgcgaaagatcatctgccgcgctttgcgagcatgccgtggaccgcgccgatttatgatagccgcagcccgaccgcggatccgcagctgatttgcatgagcggcggcgaaggcctggatgcgcagggccgctataaaggcatgagctgcacctgctataccgaacagggcaccctgtatgatattccggatggcgaatgccgccgcattgcgcgccgcggcccggtgtataacccgtatcgcgaacgcacccaggaactgggcgtggcgagccagggccaggtgaccgcgctgccgagcagccgcagcaccccgggccatgtgctggtgaccagcggccatgcgctgccgtgagtttccaagttgatttagtgaatgaatttgataatcgctgttgctgcggcggctcctgcggcgatgaggccactgccgactgccacgggataccattttgattctgttgtgagcttcagggtttctttcatcaatttttgtgtgtgcgcgttcacctcgtgaatttcagcttgt

SEQ ID NO: 9 - DNA, 5499 bp, synthetic construct
  source 1..5499: mol_type = other DNA
cgcgttgcagttgcttttgttgctggtaatgccgctttagttgctgttctgatcgctaatgcgggtcgccgcgttgctttagttgctgctatgcgctttgttcaggcaggtgttatgtttatagctgcttttcaggcgggtgttgttaatgctgcttttcacgttcaggttaatcacttttttgcgcatttatttaatgcgtttgcggctttatttcggatggctggtaattttaatttctttggttttgcgatacagcgcgctcacgcgcacgcttttttaatgatacatgctgcggcattaaatcagctgaatcagggtgctttactgcggcaggctatgggtctggcgcagaatgtttttgccgctatgcagtttgttggttttttcttttttgttctggcggccatctttttttttcacgttcagcagggtttcgttcgcatgcgggcgcggctggttgcattctttcagctgctggttctgtttcgcgcgggtgccgtttttgttgttcggggtttccagcagtttaaatttgcgcggatggttggtttatggcgcgcaaagcgtgcgtggatggcgcggattttatgagccgcgaacgccgcgcgctggaagaagcggcgcatctgtatgaaacccgccgcgaacgccatgatccgagctatgtgtttcgcgcggcgagcggcgaaaaaggcggcggcccgcatagcaacaccggccagaaaggctatccggtgtgcgtggattatctgaccgtggtgatgagccgcgatcgcctgcaggaagcgggctatctgaacgaaccgaaatttctgctgtatctgctgtttggcctgaacccggatgaagtgagcattggcaaacataccagcgtgcgctggcatttttatgcgagcagcgcgagcattctggatagcaacagcgatctggtgggcaaagtgggctgggatggcaacaacgatagcttttgcattagcctgaccggcgcggcgtgccgctatattcatgattggagcaaagcgtgccgcgtgctgaccagcctggatgcgcgcattacccgctgcgatgtggcgtatgatgattatgatggcattctgggcaccgtgcgcgatcatgaagcgcgcgcgcgcgaacatctggcgccggcgggcggctgcctgctgtttgcgagcggcggcaccccgccgcgcacccgctttctggatgatcatggcggcggcagcggctgcaccctgtatgtgggccagaaaggccataaacagctgtgcatttatgataaaggcaaacagctgggcgtggcggaaagcgcgtgggtgcgctatgaagtgcgcctgtatgcgaaacatgcggtgattccgtttgaagtgctgctggatccgatgcgctatctgcgcggcagctatgattatctgcatcagctgtttctgaaagtggtgccgggcgtgggcgaacgcattccggcggtggtgaaatatgtggaagtgaccggcgaagcgctggtgcgctggctgcgccgccaggtgggcccggcgattcatgtgctgcgcgaagcgctgggcgatctgtttccgcgctttattggcgaacatgtgagccgcgaaggcctgccgagccgctttcgcggcgtgtgcaaaaaagcgcatctgccggcgtatctgcgcgaaaccctgagcgcgcgcgtggcgagcgtggcggtgtgagtgtgcgtggtgtttttgttatctaacttaattgaggtgatatgtatgagcgtggtgcgcgtgaaagatagccatgtgctggaacgccagattaccggcaaaaccggcccgatgacctttcgcgaacagcgcgcgtgcctgctgatgggcggcggctatgaaagcgtgtttaacattgcgctgggcagcggcggcgcgtatagcccgggcgattatctgattcatccggatagctatgcgaacgataaatatggcaaactggcgtttaaccgcattagcctgattccgctgagcgtggcgctgaaagatattgtgggcaaagatgcgaaagcgagctaatgatgaccgtggcggtgctgctggtgccggcgtgccgcggcgcggatgtggatattgcgaccggcacctgcgcggcggtgatgtgggtgccgcagagcagcattctgccggaactgaccattgaaggcgcgcagtttattggcgcgggcattctgggcctgtgggcggtggcgtatgtgtttcgcctgctgcgcaaactgattaaatgaggtggaagatgctgaaacgctgcctgaccgtgctgcgccgcccgggcgtggtgggcggcctgctggcgtatggctttgtggcgccggcgtttgcggcgggcattgatgtgagcgaagtgctggaaagcattaaagcgggcctggtgccgattgcggcgattggcgcggcggtgctgagcattcatgtggcgattaaaatttataaatggattcgccagagcctgtaagagagtcagttcatctctttcatgtggacggggtgtctttgtacaggcaccccgatttaaggaggcatgatggaagggtggatctatttaggtgcgttatgtgtggcgggcctcattcttttttggggcgatgattaatgcgcgtggtgctgatgctgattctgtttgtgtttccgctgtatgcgtggggcaaaagctgcggcccggatggcaaacagggcacctatgatcatcagggcgatgcgtatcgcgaagcggtgcgcgcggcgcgctttcaggaatgcatgagcgcgggccgcaacaaagcggaaattcgcagcagctatggccattataccgtggtgatgctgaacccgtgggtgattgtgggcggctatagctattataacacctgcgcgagccgcccgagccgccagacccgccgccatccgtttaccggcagcctgggctgcaacatgggctgcctggtgagctttattcgcaacggcgatgatgaaaccagcaccgaaagcaccaccggcgaagtgtgcgataccgatcgcctggaagatgtgtgcaaaagcaaaagcggcccgcagggcaactatgtgtattttccggcgatgcatgtgtgccatccggtgcagccgccgtgcccggaaggccagcgcgcggaacatggcgtgtgcgaacgcattgatgcgtgcccgcatggcaccattaccgatcagcatacccaggcgtgcgtgccggaacaggaagcgtgcccggtgggccaggtgcgcgcgccggatggcagctgcgtgggcgatcaggataaagatcagtgcccgccgggccaggcgcgcggcgcggatggccagtgcaaagcggatcaggatcatgatggcattccggatgatgaacaggatagcgatgcgaaagataaagataaagataaagaaaaagataaaccgagcagctttagcggcggcgaacgctgcgatgtgccgccgcgctgcagcggcgatccggtgatgtgcggccaggcgcgcattcagtggcgcattgaatgcaacctgcgccgcgatgtgaaaattaccggcggcgcgtgcgataccccgccggtgtgcaccggcgaaaaatgcaacgcgatggaatatgcgagcctgctgctgcagtggcgcaccgcgtgcgcgctggaaaaaagccgcggcggcgtggtgctgggcgaaggcgatgatagcagcgcggcgaacgtggcggcgattaaagaagcgctgaccggcagcgatggcgcggtggaaggcgcggaagaaggcgatgcgagcggcgtgtttagcgatgaaagcggctatggccgcgaaggctatccgaacattaaactggatgataaaggccgcggctatagccgcgcgtgcccgcgcagcattaccgtgaacgtgctgggccatccgattgtgtttgatgcgaccccgctgtgccagtggatgcagctgggcggcaaactggtgctgctgtttgcgggcctggcgtgcctgcgcattgtgggcggccgcagcgatggcgcgggctgagtcatgccgatgtttgtggcgcgcctgctgaccggcctgctgggcctgctgaaaaccaaactgggctatctggtgatggtggcgtttgcgtggctgggcattaactggggcagctatcattttgtgatgaccccggtgattaaccagctgtatatttatatggatggcgtgggcagcagcggcggcgattggggcgcgagcgcgattgattggctgggcgtgctgaactttgatcgcgcggtgagcatgattattagcgcgtatgtggcgcgctttgcggtgctgagcggccgcctgtatctgtttaaacgcggctttggcggcaaaccgagcggcgtggcgtgccgattcatctgctgaccggcagcccgggccatggcaaaaccgcgctgatggtggaactgctgctggcggaaagcaaacgcgcggaacgcccgatttttgcggcgggcattgatggcctgcagccgggcctggcgagcgtgctgggcgatccgcgccagtggaacgcgaaagatagccagggcgattatctggtggcggcgggcgcgattatttttattgatgaagcgtggaaatggtttggccatctgcatgatgcgagccgccagccgaccccgccgcatgtgctggatctggcggaacatcgccatcgcggcctggattttgtgtggaccacccagcagaccggccagctgtatccgtttgtgcgcggcctgattggccagcatacccatgtgaaacgccgctttggcaccggctggattgatgtgtggacctggggcgaactgattgataacgtgaaaagcagcagcagccgcgaacgcgcgcagtatgtgctgcgccgcctgccgagccaggcgtatggcttttatcgcagcgcgcaggtgcataccattaaagcgcgcctgccgtttaaagtgtttctgctgccgggcgtggtggtggtggcgattgtgtgcattgtggtgagctatcgcgcgctgcgcccggcgagctttagcaccgtggtgaccggcgcgcaggaacgcccgaccgcggaactggcgaaaccgggcgcgaccgcgagcccgcgctttgtggaaggcgaacgcggcgaaggcccgcgctggcagaccgtgaccgcgtatgcgaaagatcatctgccgcgctttgcgagcatgccgtggaccgcgccgatttatgatcatcgcagcccgaccaccgatccgcagctgatttgcatgagcggcggcgaaggcctggatgcgcagggcgtgtttaaaggcctgagctgcacctgctataccgaacagggcaccctgtatgaaattgcggatggcgaatgccgccgcattgcgcgccgcggcccggtgtataacccgtatcgcgaaccgagccaggaacagggcgtggtgcagcaggcgcatcgcccggtgctgccgatgcagcgcgcgggcggcgaacatgtgattctggcgaccggccatggccagggcgtgctgcc

SEQ ID NO: 10 - DNA, 8756 bp, synthetic construct
  source 1..8756: mol_type = other DNA
aaatagacagctgcatttcaatgtgcggaaaattgtggtcttgggccgttagctcaatcggtgctgcagcgcactttcaggccgttggtcgccggttcaatgcccgcgcggcccaccagtttatccggctgtttaaattcttctttgttgccaatgctcgcatagctcacgttgctgctgctcacgcccgcgcccgcaatcagggtcgccgcggtcgcggtcaggcgttccagcatgcttttccacatcgcgcgttcggtctggctcgccgcgcgttccgcatgaattttcgcaatccacgccgggccatccagtttcgccatcgcgcacagctgcgcaatgcgttcatcgctcagcgggccgcgttcgttgcgcgcctgccacagcacctggcgggtcacgccaattttatccgccagcgctttatcgctcggaatgcagcatttttcgcgcgctttatcaatcagttcgttaatcgcgttcatgtgaagttttctattgacagcgtgtagtaattactatacattcctttttgtgtagtttttacttcacgatcctctaaggggaactcacctcccccttcatcctattctggatggggaggtgttctaagacaggatagggcacggtaggagtgttgagatgctggaatttgtggcgcgcaaaccggcggaaggcatgagctttaaaagcctgagcgatcagaacgaagtgagcgcggcgagcaaagtgtttgtgaccgatcaggaaggcgcgagcgatgcgagcagcggcattggctttctgattgcgggcattgaaaaagtgccgggcgatctgattgtggcgcagcgcaactttgataccctgggctttcgcgaatgcgatattggcatgtttaaccgccgccgcgatattaccgtgtgcattctggatgcgtgcggcaacctggaatttctgctgaaaaacattcgcgaatataaatagtctttttatttaaggtgctgccacatgagcgatctgacctttctggatgcgtatccgagctttaccagccagtatctgatttgcctgaacctgtttgtgagcgatctgcagagctatgtggatagcattgatgaaagcctgagcaaaatttttaccgatgcgagcgatgcgagcgatgaaattaccctgcaggcggtggaaagcattagccagagcctgggcgaaattgtgagcgaactgtgctatctgcgcaaacgcctgctgcatctgagcagcgtgcagagcggctgatatatgcgcattaccgataccgaacgcggcgcgcgcatggcgctgaacattgcggaaatgtatgtgcgccagctggatctgtggccggatagcctgccgcagcagtttgatttttggctgagcgtgcgcgcggcggcgctggatcagctggatgaatgctatctgctgcgccagagcctggtgtgattgatctgtggaactgcgaaaaactgagctttaaatgcctgtttctggtgcgcggcggcattgtggtggcgctgagcattctgggcggcgcgctgagctatttttttatttatctgattgtgcgctgcctgagcagcgcgctgtgctgctgagtctccctgggtgcgttatgaggtgcgtttatatgccaagcatgctgtgatttctttgtggtggcgctgagcattctgggcggcgcgctggcgtgcttttttatttgcctgattgtgtggtttctgtttagcccgtttggctgggtgctgattccgattagctttgcgttttttagcctgtttcgctatagcaaacatagctaatcaaatatagttttcttgggtgaatttttgatgatgcgctttattcagagcgtgtatagcaaatatgcgctgattcgcagctttttttatctgccgtttgcgaccctgtttattaactttctgtttagctgctttctgctgatgctggcgtgcgtgtgcctggtgctgctggtgctggtgcagccgaccagcggctttcaggattattttctgcatagcgtgagctatagcctgagcagcccggtggcgagcctgtgcctgtatacctttgcgtttagcctgtgcaactgcgtgtttcgcacctgccatctgctgtgctggggcctgtggggccgccgcgatagcctggcgtttaaccgcagctaatttttatgtggaggtgttcagtgagccgccgcagcagcagcagcggcagcgcggtgtatgtgaactttctggaacgcgaacgccgcgcggatgtggaaaaagcggcgcgcctgtatgaaacccgccgcgatcgcgtggaaccgagctatgtgtttccggcgccgagcggcgaaaaagtggtgggcccgaacagcaacaccggccagaaaggcgtggtgggcagctatccggtgagcattgattatctgaccgtggtgtttagctatgcgcgcctggcggaagcgggctattttgatgaaccgcgctttctgctgtatctgctgtttggcctgagcgtggatgatgtgattgtgggcagccataccagcgtgcgctggcatttttataacagcagcgcgagcattattgatagcaacggcgatctggtgggcaaaattggctgggatggcaacgcggatagctattgcattagcctgaccggcagcgcgtgccgctatattcatgattggagcaaagtgaaacgcagcctggcgagcctggatgcgcgcattacccgctgcgatgtggcgtatgatgattatgatggcattctgggcaccgtgcgccatcatgaagcgctggcgcgcgaacatctggcgccggcgggcggctgcctgctgtttagcagcggcggcaccccgccgcgcacccgctttctggatgatcatggcggcggcagcggctgcaccctgtatgtgggccagcgcggccataaacagctgtgcatttatgaaaaaggcaaacagctgggcgtggcggaaagcagctgggtgcgctatgaagtgcgcctgtatgcgaaacatgcggtgattccgtttgatctgctggaagaaccgatgggctatctgcgcggcagctatgattatctgtgcgaactgtttagcagcgtggtggtgagcccggtgagccgcattcgcaccgtggtgaaacatgtggaagcgaccggcgaagcgctggtgcgctggctgcgccgccaggtgggcccggcgctgggcgtgctgcgccaggcgctgggctgcggctttagcgattttattgtggatcgcgtggaacgcgaaggcctgccgagccgctttcgccgcatttgccgcggcggcgatctgccggcgtatctgcgcgaaaccctgctggattgcagcgtgggcgtgtgcgtgtaagtcattgtggatttcttttaatttaattattaggtgatttatgagcattgtgcgcgtgaaagatagcgtgctgattgaacgcagcgtgaacaccaaaaccggcccgcagatttttcgcgaacagcgcgcggcggtggtgatgggcggcgcgtatgaaaccgtgtttaacctgaaactgggcaccgcgccgctgtatccgccgggcgaatatctggtgcatccggatagctatggcaccgatgaatatggcaacctggtgctgcgccgcctgaaactgatgagcctgagcagcgcgctgaaagaatttgcgagcaaagaaccggtgagcgtggtgagcagcaaagtgagctaggagagttaaacatgatggcggcgaccctgtttgtgccggcgtgccgcgaagcggatattgatgcggtgagcaacaaatgcagcgcggtgatgtgggtgccgcagagcagcatttttccggaactgagcattagcgatgtgcagatgattggcgcggcgattctgctgctgtgggcggtggcgtatgtgtttcgcgtgctgcgcaaactgttttgaggttgaatatgctgaaacgcgtgctgagcgtggcgcgcccgagcgtgatttggggcggcattgtgagcctgttttttagcagcagcgcgtttgcggcggatgcgggcgatagcggcattagcgtggcggaagtggtggcggcgattaaagcggcggcgggcccgattagcagcattggcgtggcggtgctgagcgtgattgtggtgctgcatgtgtataaatggattcgcaaagcgttttagggcgtatttacttttttgtttgtgttaaagggtggggcgggcgtttagagtgacgccccccccctttggggaggagggggcgtatcgtgttggcttttatatttttgtttgccttgtgtattgccggcattattatttttttggattagctgcgatgcgtagtgctattttagcttttttctggtgttttgcagcagcgcgctggcggcgagcgaatgcagcgcgccgattgcgaccccggcgagcggcctgtttagcgatcaggcggaagcgtatggcgcggcgcagagcaacctgcagtatcagctgtgcctgagcaactataccaacaccggccgcgtgaaaagcggcgatcgccagtatgtggtggtgctggatcatccgagcaccgtggtgggctattatagctataaaaccagctgcagcgaaaaagaaaacgatgtgtatacctttagcgataactattatagcaactttaacaacggcacccgcagctgctttctgggctggaaaccgagcgtgaacagcagcagctgcgaagtggtgatgcatgcggatccggtgacccatcgcattaccgcggtgcataccggcaccgtgtgcgatgcgctgaactataaacaggattgcctgagccatccgggctatacctataaaagcggcggcagcggcgcggtgagcgcggtggaaggctatggcgattgcgtgccggaaaaaccgcagtgcagcctggatcagaaaaacgtgggcggcgtgtgcgtggataaatgcccggatggcatgctggaagatccggtgcgccatgaatgcgaatttgatcgcaaagattgcccgctgggccagatgcgcgcgccggatggcacctgcgtgaaaaccagcgatagctgcccgagcggccaggcgcgcggcgcggatggcgcgtgcaaacgcgataaagatggcgatggcaaaccggatgatgaacagccgggcaaagatggcgatgatggcaaaagcagctttagctggagcggcggctgcgatgtggcgccgagctgcagcggcgatccggtgctgtgcggccaggtgcgcattcagtggcgcattgaatgcaacctgcgcccggatgcgaaagtgagcggcggcgcgtgcgatagcgtgccggtgtgcaccggcaaaaaatgcgatgcgatggaatatagcagcctggtgctgcagtggcgcaccgcgtgcgcggtggaacgcagcaccgaagtggcgctgggcggcggcggcgatcgcgatccgaacgtggcggcgattaaagatgcgctgaccggcaccgatggcgtggtgaacaccggcgatgaaggcaaaccgagcagcgcgtttagcgatggcagcagcagcggcggcgatgataaagatgcgagcaaatttgatgatcagggccgcggctatagccgcagctgctttgaaccgctgaccgtggatgtgtttggcagcgaactgaccattgatctgagcccgctgtgccagttttttcagattggcggccgcctggtgctgctgtttagcgcgctggcgagctttcgcattattagcggcattagcaaaggctaagggtaatgccggtgtttctggcgaccctgctgaccggcctgtttaacctgtgcaaaagcaaactgggctatctggtggcgaccgtgtttgtgtggctgggcattaactggggcagctatcattttgtgattagcccgctggtggaacagctgtatacctatattgatagcgtgggcagcgcgggcggccgctttggcgaactggcgctgcgcgcgctgggcctgctgaactttgatcgcgcgctgaccatgattattagcgcgtatgtgagccgctttgcggtgctgaacggccgcctgtatctgtttaaacgcggctatggcgcgccgccgccgagcagcccgaaagtggaaccggtgattccgccgagcggctaatgttatgccgattcatgtgattaccgcgctgccgggcggcggcaaaaccgcgattatggtggaaatgctggaagcggaagcgaaactgggcgcgcgcccgctgtttgcggcgggcattgatggcctgcagccgggcctggcgaccgtgctggatgatccgagccagtggaacgcgaaagatgcgcagggcaactatattgtgccggatggcagcctgatttttgtggatgaagcgtggaaatggtttggcatgaaatatggcggcagcggcatgcgccagaccaccccggatcatgtgcgcgcgctggcggaacatcgccatcgcggcctggattttgtgtggaccacccagcaggcgcataaacagctgtatagctttgtgcatggcctgattggccgccatacctttattaaacgccgctttggcacccgctttctggatgtgtgggaatgggatgaactggtggaaaacgtgaacagcagcagcaaccgcgaatttagccgccatagcgtgcgcaccctgccgaaacatgtgtatagcctgtataaaagcgcggaaattcataccattcgcagccgcattccgctgcgcctgctgctgattccgctgtgcgcggtgctgagcattgtgtgcatgtggtatgcgtggcatagcctgcgcccggatagcattggcgcgaccctgctgggccagcagagcccggaaaaagcggaactggcgaaaccggtgggcaccgcgggccgcagcagcggcgcgccgagcggcgtgggccaggcgccgcgctgggcgaccgcggtggcgtatgcgcgcgatcatctgccgcgctttccgagcatgccgtggaccgcgccgatttttgatggccgcagcctgaccgcggatccgcagctgatttgcatgagcggcggcgaaggcctggatgcgcagggccattataaaggcgcgagctgcacctgctataccgaacagggcaccctgtatgatctgctggaagcggaatgccgccgcattgcgcgcagcggcccggtgtataacccgtatcgcgaacgcgtgcaggatcgcgcggcggtgcagcagagcggcctgagcggcccggtgatgccggtgagcaacgaagtgcgcagcgtgagcgtgagcagcgcgggcgcgctgccgtgagtttttgctagcatacatcatctgatgatgcaatggatgcatatatatatcttcccatcaatagtttatgaagcattcatagttatgagattcattcatattttttatctttgtcaataacgcaatttccattccagttatgattagttcataatctattcaacttatgttcataattgtatatgccgtgtagggcggcctggtttttttattgatgtggttctcagattaatgaaacagtttaataaacagggtcgccgcggtcgcgcccgccgccatcaggccgctcgccaccaccaccggataccatttgctttctttgctcactttcagggtttcttccatcagtttctgggtgcgcgcgttcatttcctgggtgcgcgcgttcatttcgttaatatgcgcgttcatttcctgggtatgggtggtcacttcatgaatttccgcctgaattttcgcggtttcgctaatcagtttcgcaatctgaatgcgctgttcatcaataatttccgcgttatcttctttctgggtcatggtcattttttctagtgttttctttgcatagtgtaaattaaacgggggtgtagggggctagccccctacgggctcgctttgcgtttctgctggcgctgaatcagcacctggcgcagcacaataatgctcgcggtttcggtcgccggatagcggcgtttgcgcagcacggttttctgcggcgcgttggtaatatgctgcggatcgctcgccgccaggccgccctgcggcgccgccgcgcgcgcttcttccatcattttctgccattctttcgccagggtgcaggtcaggctaatccagcgcagctgccattcttcaatggtgcggcgttccggggtcaccagtttgccgttaataaacgcaaagccggtccagttgccggtcagggtctgatccggaatgttatcgttaatcaccatgttgttactagtagtccgtctttaggtctcttttctagactaatcaatagccggtatagccatctttgcccgcgctcataatatgcataatctgcactttcatcacgctgctttcgcaaatgctctgcgcctggctcggggtcggataggtaatttccgcgccaatcagcagggtggtcgccgccagggtcgccagtttgcgccacaccgctttttcttcgcggctaatcgcgcgcgcttcgcccacaatgcccagcactttcgccagcggcaggccggtaatgttcgccagttccgcgcacacggtcgcatccggcaggctaatgcctttgcgatagttgctaatgctggtcgcgcgaattttcagtttctgcgccagatctttatcatatttcaccacgcacgcttctttcgctttatccagcagcaggttaatttccattttaaatccaaccctcttgatttatttgatccaatggtattgaatcatgtttgtgatccaagggcattggattcaaa

SEQ ID NO: 11 - DNA, 6357 bp, synthetic construct
  source 1..6357: mol_type = other DNA
atggcggatggcagcctggcggtgagcggcctgccgagcagcaaccgcggcgtgagcgaatttcgcaacgcggatggcaccctgaccgtgggcattgattggtttagcgcgagcgtggatctgcgcgcggtgctgggcgaagcgggcgtgtttgtgaacgatgatccggaagaagtgcgcgaatggatggatgtgaccgcggcgaacgcgcgcgcggtggcgctgcaggtgttttgctggttttttgcgggcctgggcctggaactggatgaaaccgcgggcccgggccgcttttatacctggcgcgtgcgcattaccgatcgcgatggccagcatgtgggcctgattgaactgggcggcgaaaactgccgccgcgcggatggcacctataccgcgcgcattgaactgaccggcaccggctgcggcgtggtgagcgcggcgcgctgcggccatgcgaaacgctggctggaactgcgcgcgaaactggaaagctgcgcgggccgcctgacccgcattgatgtggcggcggatgatctgctgggcaaatatccgctgaaactggcgcagagctggtatgcgagcggcgaatttgataaccgcggccagcgcccgaaagcgcagaccgtggatgatcatgatagcggcgatggcaaaaccttttatgtgggcggcaaaaaaagcgaaaaacagctgcgcgtgtatgaaaaaggccgcgaacagggcgataaagcgagcgaatgggtgcgctatgaagcgcagtttcgcgcgaccaaccgcaaagaactgccgctggatctgctgcgcgatccggcgggctatctgctgggcgcgtatccggtgctgaaatttctgcattgcgtggcgacccgcattgatattaccaaagcggcggtggatgcgacctggaaaagcgcgcgccgccatctgaaacgccagtatggcgcgaccctgaactttattctgcgccagtgcccgaccccggatgcgctgcatgcggtgattagcacctgcaccagccatcgcctgccggcgtgggcgaccgcggatgtggcgaaccagtggccggaaattgcgggcgtgaaccagaccctggaaggcgtgaccccgtgagcggcattaaagtgaccgtgctgagcgtggaagtggatgaacgcggcggcacctttaaagatgatgaaggcaaagatcgcgaatataccacccgcaaacagaaagcgaaactggaagcgggcggctttgcgtatccgctggatgtgcgcctggaaaaaggccaggcggcgtatcagccgggcgaatatgaactggatgtggaagcgatggtgaccgtgaacaaaggcgcgattaactttagcaaatttcatgcgctgcgcgtggtgaaagcgccggcgcgcgcggcggcgtaacccatggccatgtgcgtggcgctgcaggcggatggcaccctggtgccgaccggccagagcgtgggcgaatgcagcggctatgtgctggtgaccggcagcgaatatagcgtgtatgcgctggtgcaggaagcgtttgcgatgccgagcaaagaagatgcggtgacctggagcaccggctgctgcggcctggtgattgtgtggtttgtgctgggccgcctggcgggcagcgtggcgggcatgtttaacgatcgctaaatcaatcaatcaatgaaataggagagacaacatgggcgatattctgaccggcgtgagcggcgcggaagcggcgaccgcgatgattgcggcggcggcgattattgcgctggtgggctttaccaaatggggcgcgaaaaaagtggcgagcttttttggctaatggtggtgagggcagggcggcgctgcggtgtcgccctctctctttcaggggtagggcgatgatcgttctgttgttctgtggattcatgggcgcgctctgcggttgggccgccgccagggcgtgggctgcgtgcgctatattgtggtggtggcgctgtgcctggcggcgctgttttttagcagccgcgtgaccgcggcgtgcgtgcagctggaagcgccgaccagcagccataacggcgattggagctgcgcggatcagggcgaagcgtttgcgaaagtgagcagctttccggtgccggcggatctggcgaaatgcgcgatgaaaagcgtgcgcgcggtggcgagcggcccgggctttacccagcgcatgacctatccgggcaacacctgcggcattggctatgaactggatattggcaccggcagcgcgcagtttccggaagcgagcacctgcgcgaaacgcccggcgcagagcggctggaccaacccgaccgcgccgaccccgagcgatgtgtgcaacgatggctgctattatacctatgcggtggatccgggcaacccgaaaggctatagctataccccgagcggcgcgacctgcaccaccgatgatgcggcgccgccgattgatgatggcggcgatggcggcgatggcgatggcgatggcgatggcgatggcggcagcgatggcggcagcgatggcggcagcgatggcggcagcgatggcggcggcgatggcggcagcgatggcggcagcgatggcggcggcgatggcgatggcgatggcgatggcgatggcgatggcgatggcgatggcgatggcgatggcgataccccgggcgatggcgatggcaccaccccgggcgatggcgaaggcggcgaaggcgcgccgatgagcgaactgtataaaaaaagcggcaaaaccgtggaaagcgtgctgaccaaatttaacacccaggtgcgcgcgaccccgatggtgggcggcattaccgattttatgaccgtgccgagcggcggcagctgcccggtgtttagcctgggcgcgagcaaatggtggaacgcgatgaccattaactttcattgcggcggcgattttctggcgtttctgcgcgcggcgggctgggtgattctggcgattgcggcgtatgcggcgctgcgcattgcggtgacctgaggacgacgatatgcaggcgggctggtttaacgatctgaccgcgtggctgtggcgcgcggtgaaaatggtgtggcaggcggtggtggattttgtgggcgatctgtttgtgatgtggctggaacatagcctgagcgcgattctgtatgtgctgaccctgctgccgatgccggattttatgaaaggccagagcattggcggcatgctgggcaacgcgggcagcaccattctgtggtttgcggatgtgtttaaaattggcccggcgctggtgatgattggcgcggcgatggtgttttatctgctgcgccgcattctgaccgtgggcatttggtgacatgctggtgtttaacgaaggcgtgccgcgcgcgggcaaaagctatgatgcggtgaaaaaccatattctgccggcgctgaaaaaaggccgccgcgtgtttgcgcgcctgaacggcctgcgctttgatcgcattgcgaaacatctgggcattgcggaaagcgatgtgcagcagctgctggtgctggtggataccaaagatgtggcgaaactgtttgcgtgcaaacaggatgaaagcggcaaatggtgcattccggatgattttaaagatgcgctggtggtgattgatgaagtgcatgaattttatgtgaacgaacgcaaaccgctggcgccggcggtggaaaacttttgggcgctgctgggccagaacggcggcgatgcggtgattatgacccagtggattaaccgcctgcatagcgcggtgaaagcgcgcattgaaaaaaaaaacacctttcagaaaatgaccgcgattggcatgaaaggccgctatcgcgtgacctattttcataccaccagcccgggcaaatttgaaaaagtgggcggccagaccctgaaatatgatccggcgatttttccgctgtatgatggctatgcgccgggcgcggaaaacaccgaagtgtatgaagaaggcggcaaaaacgtgtgggcggcgatggcggtgcgcgcggcgatttttattgtggtgggcggcgtgggcatttatttttttgtgcattattttaccaaagatcgcagcgatccgagcaaaccggtggcggcggcgagccagaccgcgaaaccggcgcatgtgggcgcgggcctggcgaacggcgcgccgagcgtgccgattcagccgccgccgccggatccgctggcggatctgacccaggaacagcgctatgtggcggaactggcgagcaaaggccgcattcgcctggcggcgcgcgcgcgcgtgggcgatcaggatcgcgcgtgggtgcagtggattgatgatagcaacaacgtgattgaacagctggatctgacccagctgcgcagcctgggctatagcgtgagcgtggtgacctatggcgtgcgcctgagcgcgggcaaacatattatggtggcgaccgcgtggccgtggaccgcgccgattcgcgaaaaagatgcgcgcctgtataacatggcgccggaaggcagcggcggcgcggcgggcgtggcgaccgtgggcagcgatgcgggcggcgcggatcgcgatcgcgtgcgcggcggcgtgattgaatatggcccgcgcacccagggcacctttccggataacaaagcgtataccaccaacaccaccaccccggcgaccaccctgcagatgtagtttcgtgacgcgtcacgattcagcacggatcgttcggcagttcctgccagccgttgctcaggcggcgaaagcgtttgttctgaatgcagcgttcatcggtttccagcgggcgcaggcgcagggtctgcggttccgcgcgctggctgcgcggggtatacagcggctgcggcgccaccgccggcagcgccgcgctcacgctgcgcatggtaaaatagcccaggcacagcgccaccacgcccgccagcgccgcggtcatcagctggcccacaaaaatgcccagcgcaatttcccaccacaggccatcatggttgttctgcgggcgatagctcatacggcccccgttgacgatgaacgggcattgtagcggtgtaggggcatagcccctacggataacgcctcagccgcggcgcgggcggcggcggccgcggccggtcgggctgcgctggccgctcgccgccacgccctggccgctttccacgctcaggcgtttttcgcggcggcggcgcagcacatcgcgcagataaatcacttccgccgggcgcggatgccacacgcgttcgcggccttccgccatcatcagcgcccattcgcgcgcaatgttgcaggtcaggctccaatagctcatgcccaccggatcaatatcgcggccttccggggtaaaaaagcgatggccctgaaagccaaagcccgcccacgggccggtcagatccacgcgatcataggtatccagcttcactggccggtgcgtttgcgatgcaggctccagcaatgatacgccgccagcgcgcacagcagggtcagcacttcgttgcgcataatatacagcggatcggtaatcaccaccgggctcagcgccaccagcagcgcttcatgcgcgccggtatgcaccgccagcaccagcagcgccaccaccgccgccgccgcgctcaggcgatccagcacgctgcgccacagcgctttttccgccgggctcgccgccgcttccgcatgaattttcgccatccattcgccgccatccagtttcgccagcgcgcaaatctgcgcaatgcgttcatccggcatcgggttgcggcctttgcgccagttgctaatcaccgcgcgggtcacgcccaggcgctgcgccagcgcgttatcgctcggcagtttcgcgccggtgcgcgcgcgatccagcagttcgttcgcgctcggcatgtatgcgattccgttgacagactgttttgcgaacaatatacatgtcccgtgtttgcagaattgcatacaccgcgcacccccggctcccctccggggtccgcgtcaaggggcaggggataggggcttcatggataccaacaccctggcgctgctgggcgcgagcgcgctgaccgtgattgtgggcctggcgcgcctggtggcgtggattctggatcgccgcgcggaagcggcgctgcgcgcgcatcgcgaacaggtgtttattattgaaagcgtgctggaacatggcgcgccgctgagccagcgccgcgcgattgtgagcagcgcggattgccagatggaactgaccccgtgaccgcctctgtcgtctcgcatatcgacacctaccacaccgtgatcggtggccgtcgcactcggcgcttccgcctgaccgtgcgcgttgctggccggctggtagagcagagcgagtacgcctcgcgccgtgctgctctggcctgtgaagccgctgctgtggagttctacgctc

SEQ ID NO: 12 - DNA, 6966 bp, synthetic construct
  source 1..6966: mol_type = other DNA
atgagccatgaagatgcggcggtgcaggcgagcccgctgccggaacagagcctgctgagcgtgcgcggcagcgcggtgagcgatgtggaacgcgtgcgctgcgaagtggatggcctgcatcgcctgcgccgcgcggatctggaagcgcagggctatgcggaactgctgcagcgcatgcagtggcagcagttttggaccctgacctttcgcattgaagaagcgggccgcaccggcggcgtgcatccggaaaaagcggataaagcgtttcgcttttttgcgagctgcattaaccgcgaaatttatggcccgaaatggagcaccaaaagccatggcggcattcagtgggcgcgcggccaggaatttcataaagatggccgcctgcattttcatgcggtggcggcggcgccgaccgatgatctgaaccgcctgatgagccgctatcagtggcatgaattttggtttaaagaatttggccgcaaccgcattgaagcgccgcgcagccagctggatattaccggctatgtgagcaaatatgtgaccaaaggcggcgtggtggatgtgagcaaaaactttggcgcgtggaccccgccgccgattgattatacccgccgcccggtgcaggcggaatttgatcagaccacccgcaaaggcagcattgaagcgctgaaccgcggcgtgggcgcggcgccgctgcgcaccggccagcgcaaacgccgctgatcgttcaccgggacatgcagtaacgcccccggtctgggaggcaccgaagccgcagcctgttcgcctacgcgcaaccagcacggcaacgccaggaatccacccctgaagacccgccttcgaacgcaggcgacctaaagcgaccggtaggcagacaccgtgacagtcagcccccggcggtctgatgcaagcatcgcgcaagggtcatcgctacggcaccgccaccccccggcacggggggtaaggggggccttagcttgaccccacagtaccgcccgaatttcgcagtaacccaaccgacgcaagcccaaccaacagagagaacgaagaccatgagcaacgcgccgaaaattaccattaacagcgcggtggaaacccgcaccgtgaccaccagcaaaggcctgccgaaagcgatttatagccagcgcgcgaccctggaaaccgaagcgatgcgcattcagattgaagtggaatgcgatggcccggataaaggctatccggtgggcaccgtgaaagaatgggatctggtgaccgatctggtgccgggccgctttggcgtggaactggcgcgccgcatgaccctggtggatccgcaggcgggcaaagcgccgcagcgccaggcggcgtaatcgatggcggtgctgattccggcgtgcctggaagcggatctggataccgcggcgggcacctgcaccgcggtgatttggattccgcagccgagcctgctgccggaactggcggtgaaagatgcgcaggcgattggcagcgcgattgcgtttctgtgggcgaccgcgtatgtgtttcgcctgattcgcaaaaaaattcagcagagctaggaggacatgcaatgcgcaaactgaaaaccctgtttaaaaacaaaaccgcggcgctggcggcggtgggcagcgcggcgctggtgagcgcgccggcgtttgcgagcggcggcggcggcgtggatgtgggcgatgtggtgagcgcgattcagggcgcggcgggcccgattgcggcgattggcggcgcggtgctgaccgtgatggtgggcattaaagtgtataaatgggtgcgccgcgcgatgtaacgaccaccggcggacagggccaactccctcccgccggtcttttatggggatagggcaggggaacgggcgatggaagggtggatttggttgggcgcatggctggtggcctgcgcgattgtcttcgtggatttcgaataatgggctggctcgcacgcgtgtttgcatccgcgattgcgcgtcgcctcgcatacgtgctggtggcggcgaccctggcgtggtgcggcatgggcaaagcgcatgcggcgaactgcgatgcgaccagcgatatgtgcagcgaaggcgatgcgtggaacgcggcgcgccagctggcggatagccgcggcccggatacctgcaaactggtgggcggcaacaacgcgagctataccggcccgaacattgaaaaagatccgggcaacagcagcaaagatgtggcggcgctggcggtgcgcgtgagctgcagcaacggcagcctgccgtttctgggcaccaaatattatgtgaccgcgaaaagctgcgaacaggaaccgggctataccggcggcggcccgtggggcacctatgtgggcaccgcgcgcaacggcagcattggctgccgcaacggctgcgatggcgtgtggtttggcaacggcgatggcagcatgacctggggcagcaccggcgcggtgtgcccgaccgatccggaaaaaacctgcgatgcgatgggcaaaggctatggctggaacggctatctgggcgtgtgcgaaccgccgccgaccgaagaatgcccggaaggccaggtgccggatggcaaaggcggctgcagcaccaacaaatgcccggaaggcatgctgctgcaggcggatggcacctgcgcgccgaaaaaaaacgattgcccggcgggcatgattaaaagcccgaccggcagctgcctgccgggcgatggccagtgcgcggcgggcgaagtgcgcggcccggatggcacctgcaaaaaagatgcggatggcgatggcgaaccggatggcgatggcgaaggcggcgaaggcgaaggcgatagcaaaagcgaatttagcggcggcgatagctgcgatagcccgccgagctgcagcggcgatgcgattatgtgcggccaggcgcgcattcagtggcgcattgattgcaacacccgccgcgatgtgaacattaccggcggcagctgcgcggcgatgccggtgtgcgtgggcaaaaactgcaaagcgctggaatatagccagctgctgatgcagtggcgcgcggcgtgcgcgctggaaaaagcggcgaacaacagcggcggcggcaccggcaacaacgcggatgtgaaagcgattcgcgatgcgattaccggcaacggcaccgcggatattggcgcggatggcaaaccggcggatgcgtttagcgatgaaagcggctatggcgaagatggctatccgaccggcgaactggatacccagggctttggctatagccgcacctgcccgaccattccggatgtggcggtgtttggccagaccctgcattttgataccagcaaattttgccagtggatggtgctgggcggccagattgtgctggtgatggcgagcctggtgagcctgcgcctgatgagccagggcggcagcgcgtaatgccgtggctgattgcgcagctggtgaccgcgctggcgtggctgtttaaaagccgcattggcctgtggattatgaccgcgctggtgtggctgggcattaactttggcaccattaaaatggtggtggaaccggcgattgatctgctgaaagattatgcgcagggcatgggcaacggcaacggccagctgggcgcggatgcgatggcgtggtttggcgtgctgcagtttgataaagcgctgaccatggtgattagcgcgattgcggcgaaacatgcgattatgcagggccgcctgtttctgtttaaacgcggctttggcgcgaaaccgtaatgccgattgaactgtataccggccagccgggcaacggcaaaaccgcgctgatgatggaacgcctggtggaagaaagcaaacgcgcggaacgcccgatttttgcggcgggcattgatggcctgcaggatggcctggcgaccgtgctggatgatccgcgcaaatggaacgataaagatgcggatggcaactatgtggtgccgaacggcagcctgatttttgtggatgaagcgtggaaatggtatggccatctgcatgatgcgacccgccagcagaccccgaaacatgtgctggatctggcggaacatcgccatcgcggcctggattttgtgtggaccacccagcagccgaaccagctgtatccgtttgtgcgcggcctgattggcgcgcatacccatgtggtgcgccgctttagcaccaaaatgattgatgtgtttcgctggggcgaactgaacgaagaaattaaaagcagcgcgaaacgcgatctggcgcagcgcaccacccgcctgctgccgagcagcatttttggcgcgtataaaagcgcggaagtgcataccattaaaccgcgcattccgtggaaagtgctggcgctgccgggcctggtgattctggcgattgcgctgggctggctggcgtataccatgctgaaaccgagcgcgatggcggcgaaactgggcgataaaggcacccagagcgcgctggcggatgcggcgccgggcggcagcgcgaacagcgcgcgccgcgatggcccgcgctgggaaagcccgaccgaatatgcgaaacagcatctgccgcgctttggcaccatgccgtggaccgcgccggtgtttgatgatcgcagcattaccgcggatccgatgctgatttgcatgagcagcctggcgggcaccgatgcgcagggcaaacataaagaagcgagctgcacctgcatgaccgaacagggcaccgcgtatgatctggatcagccgcagtgccgcaccattgcgaaacgcggcccggtgtataacccgtatcgcgaacgccgcgaaaacgaacagcagccggcgcagcagcagcaggcggtgcagggcggcgcggcggcgccgggcctgagcggcgtggtggtgcagcgcggcatgcgcacccagggcacctttccggaaagcaaaggctataacaccaaaaccaccaccccgagcaccagcctggaaatgtgacatgaccagcagcggccgcgaagcgctgaaatggattgcgctggtgctgatgaccggcgatcatgtggcgaaagtgttttttggcggctatgtgccggtgctgagcgaactgggccgcattgcgtttccggtgtttgcgctggtgatggcgtataacctggcgcagccgcgcgcggattatgcgaaaagcgtgctgcgcctggcgggctggggcctgctggcgcagccgtttcatgcgtgggcgtttggctattggctgccgctgaacgtgctgctgacctttgcgctggcggcgctgctggtgtggaccgtgcatgcgcgccattggctgtatgcggcggtgtgctgcctggtgctgccgctggtggtggattatcagtggagcggcgtgtgggtggtgctgaccgcgtggtgctggtttcgcaccggccgcctggaagcgtttggcggcgtgctggcgaccatggcggcgctgtgctggtataacggcaacctgtgggcgctgggcgcgctgccggtgctggcgctgggctatgtgtggtggccgctgccgcgcctgcgctgggcgttttatggctattatgtgggccatctgggcctgctggtgtttattgcgagcctgccggcgtttcagcagcatgtggcgtaggtagatgaccctgtttccggcggaaattcgcctgccgggcaccctggtggcggtgggcgtgagcggctttgcggatctggcgctgcgcagcgtggcgattattaacgatcatagccgcgcgatgcagcaggtgagcgatcagaaccgcatgagccatggcagcatggtgctgccgagcggcgtgaaaaaatgatgggcctggaaaccaaaaccggcccgcgcgcgggcaaagtgtatgcggtggtgggcgtggatcatgcggcgattagcgtgagcctgggcccgcgcctgggcagccgcctggtgattcatagccatacccgccgcgcgcgcatttttagcgcggcgagcgcgagctttgcgtaatgaatgctcgggctcgcgttgttcgcttcaaacgcatgatgaatcgccgcttccgcgcggcccggcaggctcacgcccagcagcagcagcaggcccaggctcacgcccgccaggcgctgcgcaatgcgtttccacgccgcgcgttcatcttcgctgttgctgcgttccgccatcaccagcgcgctccaggtttccgggttatcgccaatatcttccgccatgcgttcaataaactgaatttccgcgtttttgccttttttccagctgctcaccgccgcgcgggtcacgcccagcgcaatcgcgcccgcgttatcgctctgaattttctgcacatgtttccagcggcaaaacagatcatagctctggctcatgtaattaccctattgacaccatgtcatgacccagcatacagttgaccctagatgtcagcagagtcttgacatcgcccacccgccggccccgcccggtgccggttgggcgggttctaccgggcaccgggcaggggatacaccgtgaaagcacttatccagttttgccgtgaattcggtctgcttgtttgcatggcatgcgctaccgggtttggcattggcggactcgttggctttatcgctggtcagggcgctgcaagtagcgttgaacaccagattcagcgcgatgaggcgctccagcagctttgcatcgacggtaacgaaggcgcctgccgcgttatggagttccgcagatgagccgcccgaccgataccgaacgcggcgcgcgcattgcgctggattatgtggaaagcaaactgattcagcgcgatctgtttccgagccgccgcaccccgccgctgaaattttggcgcgaaattaaagcgattgcgaccgaacatctggcggaatgcaaagcgctgcgcgaagcgcgcgcgtgaacacgggcgacctgaaattctggcagctgatcgagctgaaaaagaaagccgaaaccgacgacgaacgcgccgagctagaccgactgatagaggaacgcatcaggtgggtaggctcggggcagggcg

SEQ ID NO: 13 - DNA, 7194 bp, synthetic construct
  source 1..7194: mol_type = other DNA
ttcgcataatatacagcggttccgccatcgcggtcggcgcaaagccggtcaggctcgcttcatgcgccagcggggtcaccgccagcaccagcagcgccaccaccgccgccgccgcgctcaggcgatccagcacgctgcgccaagcgcgaccgatgaactgctggataaagtgaaagaaagctgcagcctgccgagcgatatggcgctggcggaaaaactgggcgtgaaccgccagatgatttggaaagcgcgccatgaagataaaccgctgagcgatgaacgcattgcgcagctgtgcgcgatggcgaaactggatggcggcagctggatggcgcgcattcatgcggaacgcgcggcgagcccggcggaaaaagcgctgcatgtgaccctcagggttgacatcgtgtgacccaccgagtttacatgcgcttgcgttacctcattgggtaacgccgcgcacccccggctcccctccggggtccgcgtcaaggggcaggggagggggcttcatggatgtgattgcgtttgcgctgattggcgcgagctgctttgtgtgcagcattggcaccgcgcgcctggtgagctggattctggatcgccgcgatgcggcgattctgcgccagtttcgcgaacaggatctgattgcgcgcgcggcggcggaagtgcgcaacgcgtaacctcgattggtcccagttcacgtggttggatcacgtgattgccatcttctctctcgcggtctactcctttgcggtcgctgcctttctcaccgcgccgtggtggttgacccgtttgttcaagggtggccgatgagcgcgggcacctgcagcttttgcggcgataccaccgcgtatttttttccgggcggcctgtgcgtggcgtgcaccagcaacaacgcgcgcattcgcatgcaggaacagccgacccagagccgcgaactgagcgcgtttgatgcgagcgtgggcgtgatgcaggcggcgacccgccgcaccgaaattgcggcggaaaaaattcagaaaaacaaacgcgtggtgggcaccagcgtgcgcgaatttgatgcggcgcatccgattgcgctgaccgcggaaggccagcgcgcggcgctggcgctgggcctggtgcattataaaaccagcgatacccgcgcgagcagcaccggcaccgtgaccattgaaattgatccgctgcaggcgcgcgcgcagcgcctgcgcaaaagcgtgattaccggcgcgcgcctgcatgatcaggaagcgaaaaaaggcagctttcgcggcgcgtggtattttctgaccctgacctatcgcgatggcagcgatagcagcccgcgcgatgtgagcgaactgtttaaacgcatgcgcggccattttaaccgcctgaaaagcggccgcgcgcgctggaaccgcgaaagctttcgctatgtgtgggtgggcgaactgacccagcgctttcgcccgcattatcatgtgatgctgtgggtgccgaccggcatgtattttggcaaagtggatcagcgcggctggtggccgcatggcaccacccagattgaaaaagcgcgcaactgcgtgggctatctggcgaaatatgcgagcaaatttaccgcgctgaccgcgggcgcgtttccgaaaggctttcgcacccatggcattggcggcctggataccgaaagcaaacgcgaactgcgctggtggaaagcgccgaaagatgcgcgcgaagcgctgggcggcgaagcggatattcgcaaagcgaaaggcggctggtttgatcgcctgaccggcgaattttggccgagcccgtggaaagtgacctttatttttggccgcacctttgcgtggaaagtggtgccgctgtgaaagtgcagattatgagcagcgcggtggcgattcgcagctttccggcgcgcgatggcaaaccggcgacccattttcgcgaacagaccgcggcggtgctgcgcgaaggcgattttccgctgccgtttaccattagcctggatgaagatcaggcgccgtatgcggaaggcttttatgtgattgatccgaaaagcatgcagaacaacaaatatggcggcctggaatttggccgccgcattcgcctgattccggatagcgcggcgaaagcggtgcagccggcggcgcgcgtggcgtaacccatggcggtgctgattccggcgtgcctggaagcggatctggataccgcggcgggcacctgcaccgcggtgatgtggattccgcagccgagcctgctgccggaactggcggtgaaagatgcgcaggcgattggcagcgcgattgcgtttctgtgggcgaccgcgtatgtgtttcgcctgattcgcaaaaaaattcagcagagctaggaggacatgcaatgcgcaaactgaaaaccctgtttaaaaacaaaaccgcggcgctggcggcggtgggcagcgcggcgctggtgagcgcgccggcgtttgcgagcggcggcggcggcgtggatgtgggcgatgtggtgagcgcgattcagggcgcggcgggcccgattgcggcgattggcggcgcggtgctgaccgtgatggtgggcattaaagtgtataaatgggtgcgccgcgcgatgtaacgaccaccggcggacagggccaataccctcccgccggtcttttttggggatagggcaggggaacgggcgatggaagggtggatttggtgggccgcatggcgggcggcctgcgcgattgcctgcgcggctttcgcattatgggctggctggcgcgcgtgtttgcgagcgcgattgcgcgccgcctggcgtatgtgctggtggcggcgaccctggcgtggtgcggcatgggcaaagcgcatgcggaagattttccgacccagggcgcggcgtatagcgcgtgcatgagccagaccgcggcgtatattgtgagccgcccgcgcccggaactggcgcgcaacccggaatgcgtggtgctgcagggcagcaaagcgtatcgcggccagtatgaacagaaagattgcaccagctgcgaatggggccgcgcgtattataacacctttggctggattgcgggctgcgatgcggaaccggattttaccggcggcggcccgtggggcacctatgtgggcaccgcgcgcaacggcagcattggctgccgcaacggctgcgatggcgtgtggtttggcaacggcgatgataccatgacctggaaagcgctgggcggcgtgtgcccgaccgatccggaaaaaacctgcgatggcatggcgggcaactatgcgtggaacggctatctgggcgtgtgcgaaccgccgccgaccgaagaatgcccggatggccaggtgccggatggcaaaggcggctgcagcccgaacaaatgcccggaaggcatgctgctgcagcaggatggcacctgcatgccgaaaaaaaacgattgcccggcgggccagattaaaagcccgagcggcgcgtgcctgccgggcgatggccagtgcgcggcgggcgaagtgcgcggcccggatggcacctgcaaaaaagatggcgatggcgatggcgatccggatgaaccgggcgaaggcgataaaagcagctttagcggcggcgatagctgcgatagcccgccggcgtgcagcggcgatgcgattatgtgcggccaggcgcgcattcagtggcgcattgattgcaacacccgccgcgatgtgaacattaccggcggcagctgcgcggcgatgccggtgtgcgtgggcaaaaactgcaaagcgatggaatatagccagctgctgctgcagtggcgcaccgcgtgcgcgctggaacgcgcggcggtgggcggcggcggcggcagcggcagcgatgcggatgtgaaagcgattcgcgatgcgattaccggcaacggcaccgcggatattggcgcggatggcaaaccggcggatgcgtttagcgatgaaagcggctatggcgaagatggctatccgaccggcgaactggatacccagggctttggctatagccgcacctgcccgaccattccggatgtggcggtgtttggccagaccctgcattttgataccagccgcttttgccagtggatggtgctgggcggccagattgtgctggtgatggcgagcctggtgagcctgcgcctgatgagccagggcggcagcgcgtaatgccgtggctgattgcgcagctggtgaccggcctggcgtggctgtttaaaagccgcattggcctgtggattatgaccgcgctggtgtggctgggcattaactttggcaccattaaaatggtggtggaaccggcgattgatctgctgaaagattatgcgcagggcatgggcaacggcaacggccagctgggcgcggatgcgatggcgtggtttggcgtgctgcagtttgataaagcgctgaccatggtgattagcgcgattgcggcgaaacatgcgattatgcagggccgcctgtttctgtttaaacgcggctttggcgcgaaaccgtaatgccaatcgagctatacaccgggcaacccggcaacggcaaaacggcgctcatgatggaacgcctggtggaagaaagcaaacgcgcggaacgcccgatttttgcggcgggcattgcgggcctgcaggatggcctggcgaccaccctggaagatccgcgccattggaacgcggtgaaaaccggcgaagtgtgcacctgcaacgataccagcgcgcaggcggaatgcaccgcgcatgtggtgccgaacggcagcctgatttttgtggatgaagcgtggaaatggtttggccatctgcatgatgcgacccgccagcagaccccgctgcatgtgctgcagctggcggaacatcgccatcgcggcctggattttgtgtggaccacccagcagccgaaccagctgtatccgtttgtgcgcggcctgattggcgcgcatacccatgtggtgcgccgctttggcaccaaaatgattgatgtgtttcgctggggcgaactgaacgaagaaattaaaagcagcgcgaaacgcgatctggcgcagcgcaccacccgcctgctgccgagcagcatttttggcgcgtataaaagcgcggaagtgcataccattaaaccgcgcattccgtggaaagtgatggcgctgccgggcctggtgattctggcgattgcgctgggctggctggcgtataccatgctgaaaccgagcgcgatggcgggcaaactgggcgataaaggcacccagagcgcgctggcggatgcggcgccgggcggcagcagcagcaccgcgcgccgcgatggcccgcgctgggaaagcccgaccgaatatgcgaaacagcatctgccgcgctttggcaccatgccgtggaccgcgccggtgtttgatgatcgcagcattaccgcggatccgatgctgatttgcatgagcagcctggcgggcaccgatgcgcagggcaaatataaagaagcgagctgcacctgcatgaccgaacagggcaccgcgtatgatctggatcagccgcagtgccgcaccattgcgaaacgcggcccggtgtataacccgtatcgccagcagcgcggcaacgaacagcagccggcgcagcagcagcaggcggtgcagggcggcgcggcggcgccgggcctgaacggcattgcggtgcagcgcagcacccgcacccagggcagctttccggaaagcaaaggctatagcaccaaaaccaccaccccgagcaccaccctggaaatgtgacatgaccagcagcggccgcgaagcgctgaaatggattgcgctggtgctgatgaccggcgatcatgtggcgaaagtgttttttggcggctatgtgccggtgctgagcgaactgggccgcattgcgtttccggtgtttgcgctggtgatggcgtataacctggcgcagccgcgcgcggattatgcgaaaagcgtgctgcgcctggcgggctggggcctgctggcgcagccgtttcatgcgtgggtgtttggctattggctgccgctgaacgtgctgctgacctttagcctggcggcggcggtggtggtgctgctgggccgcgtgattggcattgaaagcagcaacgaaagccagcgccgcccgtttctgctgctgctgctgggctttctggcgccgctggtggtggattatcagtggagcggcgtgtggctggtggtgaccgcgtggggctggtttcgcacccgccgcggcggctggctgctgctggcggcgggcagcatggcggcgctgtgctgctataacgcgaacgtgtgggcgctggcggcgctgccggtgctggcgctgggctatgtgtggtggccggtgccgcgcctgcgctgggcgttttatggctattatgtgggccatctgtttgtgattgcgctgattgcgagcctgctggcgtgatttagcacggatcatgcggcagctgcacccagccgttgctcacgcgtttaaagcggcggccctgcaggcagcgttcgccatccgccagcgggcgcggcggatcatattcgcgcatcagcggcgcctgttccgcgcgctgcacgttctgggtcagcgcgcgctgcatctgctgggtcgccagtttgctttcgcgttccagctgggtcatcgcctggcgcgcttcatagcgggtatacaggcccaccacaatgctatgcgccagcagcgcaataaagccgcccagcgcaatctgccaccacagatcgctgctttccgcttcgcgatagcggcgttccatgccgcctccgatttgtgagcgtgaattgtagactcagggtgtaggggcgacgcccctacggataacgccttagccgcggcgcgggccgcgcgggccgcgggtgcggcgcaccacggtgctgcgatccgcgctgcccgcgccatccaccacgctaatgcgcatttcgcggcggcggcgcagcacatcgcgcagataaatcacttccgccgggcgcgcatgccacacgcgttcgcgttcttccgccatcatcagcgcccactcgcgggcgatgttgcacgtgagcgaccagtagcgcattcccactgggtcaatgtctcgaccctctggggtgaagaatcggtgcccttgaaaaccaaaaccggcccaagggccggtcaggtctacgcgatcgtaggtctctagcgtcactggccggtgcgtttgcgatgcaggctccagcaatgatacgccgccagcgcgcacagcagggtcagcacgccgtttcgcataatgtata

SEQ ID NO: 14 - DNA, 8406 bp, synthetic construct
  source 1..8406: mol_type = other DNA
ttcgcataatgtatcgtttggcgcatgtttaaaggtctgaatctggctcgctttcgcatgatccggcagcgcggtgccaatgcaaatcagcagcgccatcgccgcgcccgccagctgtttatacagtttcgcccattcgcggcccagttcgccctgatcctgttcgctatgaatcagcagcagccacgggcccgcatcctggcccgcaattttcgccagctgctgaatgcgttcatccggaatcggtttgttgcccagtttccactggctcagcacgccgctgctaatgcccatctgttccgccaggccgcgcaggctgccgcgcaggctgcgctgaatcgccagttccaccagggtgttaatatgcatttgcgcgttccaaaaggtatgcagtgcttacctttaaggatacactgcaagtgcatcccaaatgggctgcacccagcccctcgcacccccgagggtgtctgggttcaacgggtaggggtaacggggttacaccatgagcagctatcaggtgcagtatcgcccgctgatgggccgcatgtggatgaccctggatctgtttctggatcgcaaaagcgcggtggtgcgcatgcatgatcgcgtggatgcgatggtggcgcgcggctttgaacgcaacgatgtggcgcgcgattttcgcgtgcgcgcggtggaacgcgtggcggcgtgaatatcgaccagtatcagttgcgagattacgctctcactattcgtcagcgttggtatcaggactacgcgcgtgcccgctcccagcttcggttgtgggctgtcatggatgagcgacttagttcttccgatgtgccgtcgtgggttgatctatctttatctgtttatccattgcgtttatctggtgacatgttgggttggtgtgcttctatcggcccacgtcaccttcataaacaccgcatgctgcaattccggggtcgtgtgaggttgcctgcatgaacctggatcagtataaagcgattcagccgcgcattgtggaactgcagcgcgcggcgctggattttgcggaactgggcgaaagcaaagtgagccgcctgattctggcggcgtgctttgcggcgcagaaactgaaaccggcgaaaccggcgagcggcgatcagctgccgctggaaccgaacccggtggtgaaactggcgaaaggcaaaggcgcgtaacgcgcccttttttgggggtaattaataatgatggcggtgtttctggcggcgctgggcgtgtttagcattctgttttttctggcgggcgtgattcgcattttttgctggatggatcagcgctatgcggcgaaactgcaggcggaatatcgcgcgtatgtgatggcggaagaagcgaaacgcgaatttcagcgcagcctggcgtatcagcgcggcgtgttttatgatccgtatgattgatacgacagaatctccgggggtacacaagtgatccgctggtacataactctcctgctgtccatatattttctttctcaatctcatcagccgatatcggcaacgttattctttctgggacttctgttcctctgcgtctatcgcctcggcggtaacaagctgctgcggatgatgcggatgcggatccgtgcatcgcgccgtcatacgtcaaacggggggcatcgcaatgcgcgaaagcaaaaccaccgtgccgcgcgcgcagcgcgtgctgctgagcgtgccgaccccgcgcgcggatgtgtttgatctggcgggcgtgctgattaaaattgatggccatctggtgaccctgggcgattattgggtggcggtgcgcatgcaggcggcgagcgcgcgcctggatcgcggcgtgggcgcgcagccgctgcgcgcgaccccgtatccgcataccggcaacgtgtgcctgattggcaaccgcccggcggtgccgagcagcgcgagcccgagccatacccatctgatgaccgattttcatctgtgccgcctgggcggcaccccgtgagcgcgctgggcgaatttagcccggatggcccggtgaaaaacaccggcctggtgggcccgggcagcaacaccggccagaaaggccagccggcgcgcgatcgccagcatgcggtgaccgtggattttctgaccgtggtgtttaaccgcgatcgcctggaagcggcgcgcaaactgaacctgcgctatctgctgcagtggatttttggcctggatccgcgcgatgtgtttatgggccaggtgcatgcgaaacgctggcagttttatcgcagcagcgcgccgattattgatgcgaacggcgaactggtgggccgctttggctgcgaaggcaacggcgataccatgtgcgtgagcctgagcggcgcggcgtgccgctatattcgcaactggcgccatgtggaagtgaacctgcgcctggtgggcgcgcgcgtgacccgctgcgatattgcgtatgatgattatgatggcgtgctgggcaccgtgcgcgatcatgaagcgcaggcgcgcgcgaacatgagcgataccggcggctgcctgctgtttggcagcggcccgggcgcgccgccgaaaacccgctttatggatgatcatggcaacggcgatggcagcaccctgtatgtgggcggcaaaggccataaacagctgtgcatttatgaaaaaggcaaacagctgggcgtgtgcgaaagcccgtgggtgcgctatgaagcgcgcctgtatggcaaacatgcggaactgccgctggaaatgctgacccatccgatgcgctatctgcgcggcagctatgcgtatatggatcgcctgctggcgaccgtgggccagggcgtggcgaccgcggtgaaaattagcaaacgcaccgtggaagcgaccgcggaagcgatggtgcgctgggcgcatcgccaggtgggcccgaccctgcattgcctgcgcgaagcgctgggcgatcagtttggcgattttgtggtgaaccatctggcgcgcgaaggcctgccgagccgctttaaacgcgcgtgcaaagcggcggatctgccggcgtatgtgcgcgaaaccctgattaaacgcgaaattccgtgagcattgtgcgcgtgaaagatgatcgcattattgaacgccgcgtggtgattaaaggcgtgccgcagatttttcgcgaacagcgcgcgtgcgtgctgctgggcggcggctatgaaaccacctttaacgtgggcctggcggatggcccgtgctatcaggtgggcgattatgtgattcatccggatagctatggcaccggccagtatggcgatgtgatgctgaaacgcattaaactgtggccgctgggcgtggcgctgcgcgaaaccgcggcggcggtgccgccgccgagcaaaggctaaaggctgcctgatcgccatggcggtgtttgtgccggcgtgcctggaacgcgatctggatacccagaccggcacctgcagcgcgccgatttggattccgcagccgagcctgctgccggaactgaccgtggcggatgcgcagagcattggccaggcgattgcgctgctgtgggcggtggcgtttgtgtttcgcctgattcgcaaaagcctgcagcagagctgaggattacaaaatgtttaactttaaaaaatttccggtgattgtggtggcggcgagcattgcgaccattgcggcgccggcgtttgcgagcggcggcagcagcggcgtggatgtgagcgcggtggtgagcgcgattaacgcggcgagcaccccgattgcgagcattggcgcggcggtgctgattgtgatggtgggcaccaaagtgtataaatggattcgccgcgcgatgtaacggctgccggcgggcagggccgactccctcccgccggtctttagggggtggtatggaaggttggatttggttgtgcgcgtggctggtggcctgcgcgatattgttcctggggtctgattgatatggcgcgccgcggcattctgatgctgctgattattgcgatttgggtgatggcgctgtttattcgcgatgcggatgcggcggtgtatccggatcagggcagcgcgtatgcggcgtgccaggcggcgggcgcggcggcggcgagcgatccgaacgaatttccgagcggcaactatcgctgcgaaggcggcgcgaacccggatgcgggcggcagctatcagtgctattttcaggcgcgcatttatggcccggatcatgcgtattttgatagcattgattgcggcatttatacctttccggcgaacagcagctgcgcgagcaaaaaaagctttaccaccacctttagcccgaaagcgggcagctttagctgcaacgatggctgcattaccatgttttatccgaacctggatggcaccaacaccgcgagcccgaccggcaacacctgcgatccgcataacctgaactgcggcgatggctttctgtggaacccggtgctgaacgtgtgccagccgaccaaaccggaatgcccgagcggccagaaaccgaacagcctgggcaaatgcgcgccggaaccgtgcccggatggcatggtgcagcagcaggatggcacctgcaaaccgaaagaaaacgaatgcccgcagggcgaagtgaaaggcccggatggcagctgcgtgaaaaaaccggatgtgtgcggcgcgggcaaagcgatgggcagcgatggcacctgcaaagatgataaagatggcgatggcaaaccggatgatgaagaaggcgatgataaagataaagataaagatggcaaagatgatccgagctttagcggcggcgatagctgcgatgcgccgccggcgtgcagcggcgatgcgattctgtgcggccaggcgagcattcagtggcgcattgattgcaacacccgccataacgtgaacattgcgggcggcgcgtgcggcaccccgccggtgtgcaccggcgataaatgcgatgcgattcagtatgcgagcctgctgatgcagtggcgcaccgcgtgcgcgctggaaaaaggcaccagcagcagcggcggctttggccagagcgatagcgataacctgagcgcgattaaaggcgcgctgaccggcaacgcgggcaacccggatattggcgatccgggcgatccgagcggcgcgtttaccgatggcagcaacagcaccaccggcgatggcaaactggataccagcggctttggctatggccgcagctgcccgaccattccggatgtgagcgtgtttggccaggcgctgcattttgataccagcccgttttgcagctttattgtgctgggcggccagctggtgctggtgtttgcggcgctggcgagcctgcgcattattagcagcggcggcgatagctgatgccggcgctgattggcgcgctgattagcgcgctgagctggctgtttcgcagccgcattggcctgtggattatgaccgcgctggcgtggctgggcattacctttggcaccattaaaattatgattgatccggcgattgatctgctgaaaggctatgcgcagggcctgggcggcagcggccagctgggcgcggtggcgatgcagtggatgggcgtgctgaactttgatcgcgcgctgaccatgattattagcgcggtggtggcgaaacatgcgctgctgcagggccgcctgtttctgtttaaacgcggctttggcgcgaaaccgtaatgccgattgaactgtttaccggcctgccgggcaacggcaaaaccctgctgatggtggaacatctgagcaaagcggcggaacagggcgcgcgcccgatttttgcggcgggcattgatggcctgaaaccgggcctggcgagcgtgctggatgatccgcgccattggaacaaccgcgatgcggaaggcaaccatctggtgccggatggcgcgctgatttatgtggatgaagcgtggaaatggtttggccatctgcatgatgcgacccgccagagcaccccgccgcatgtgctggcgctggcggaacatcgccatcgcggcattgattttgtgtggaccacccagatgccgctgcagctgtatccgtttgcgcgcgcgctggtgcagagccatcatcatgtggtgcgccgctttggcaccagcctgattgatgtgtttacctggcaggaactgcaggatgatgtgaaaagcaccccgcgccgcgatgcggcgctgcgcaaaacccgcattctgccgaaagaatattttgcggcgtataccagcgcgaccatgcataccattaaacgcaaaattccgctgcgcgtgctggtgctgccgctgctggtgattgcggcgattgcgtgcgcggtgattggctatcgcgcgctgcgcccgagcaccatgaccgcgaaaattaccggcgcgggcccgagcgcggcgagcgcggcgccgggccaggcgggcgtggcggcgggcgcgagcaccgaacgccattttgcggatgcgaccgattatgcgaaacagcatctgccgcgctttccgaccatgccgtggaccgcgccggtgtttgatgaacgccatgtgaccgcggatccggcgctgtattgcatgagcagccgcccgggcctggatgcgcagggccagcatagcgattttagctgcacctgcattaccgaacagagcaccatttatgcgattagcgatggcgaatgccgccgcattgcgcgcatgggcccggtgtataacccgtatcgccagcagcagcgccagcaggatcagccgcagcagcagagccaggcggcggcgaccatgccgatgacccgcggcggcgtgattgaacgcaaagcgcgcggccagggcacctttccggaaagcccgggctataaagcggataccgcgaccccgccgaccaccctggatatgtgatgaccagcggcggccgcgaactgattaaatggattgcggtgctggcgatgaccggcgatcatgtgattaaagtgtttcatctgggcgatgtgccggtggtgagccaggcgggccgcgtggcgtttccgctgtttgcgatggtgctggcgtataacctggcgcagagcggcgcggataccgcgaaaagcctgcgccgcctgctggcgtggggcgtggcggcgatgccggcgtattggctggcgtttggcgtgatttgcccgtttaacgtgctgctggcgtttgcgctgggcgtggcggcgattcgcgcggtgcagggccgccgctgggtgctgctggcggtgtgcgtgctgccggcgccgctgtttgtggattatcagtggagcggcctgagcgtggtggtgggcgcgtggtggtattttcgcagcccgcgcccgaccatgctgatgggcattctgtgctttgcggtgccggcggcggcgctgtgcgtgtataacggcagcgcgtggagcctgctggcgattccgatgctgcgcctgggcgatgtgccgattgcgctgccgcgcacccgctgggcgttttatggctattattgcggccatctggcgctgctggcggcgctggcgcgggttgcgcagctgctggcgcgcatgtttcacgcgctgcaggcggcggccctgcatgcagcgttcgcccggcgccagcgggcgcggggtatacaccgccggctgaattttttcgttccacagaatgcggcgggtgcgttccagttccgccgcgctttcaatttccatttctttgttaaatttgctaatctgcgctttaaaggtcgcttcttcgctcggggtcatcgggcgcagaatttcgttggtcatcgccgccgcctggcggcgcgcgttccattcaatcaggcccatcgccgccgcaatcagcaccaccacgctaatgcccgctttcagccagccttccatgccatcatctttggttttgcgggtcggttcgctataggtgcgcgggcgaaattccagttcggtcacatcggtatctttgcccagggtcggttcctggcgttccatggtgccgccttttttcatctgcgccatggtcggcgccggcgccatgcccctgcgtagacgctttagcccgccattttgccaaaatggcgatcgcgccagttcgccagatccaccaccaccactttcaccatcgcctgctggcgcgctttgcgcgctttgttgcgcgcgcgcgcggtatcgcgcagatcggtcgcatccgcgcgccacagcaggccgcgcaggcggcgttccggaatgcgttcgccggtcggcgccaccagatcgcggcccgccaggcgccagcccgcccacgggccatgcagttccacatggttatcaataatatggcgcgcatgcgcctgcgcgcaatggttcgggcacggtttgcccgcttcccagcacggcgggcggcgatcaatgttataggtatcgctcacgccgcaatttctttatcgctcggcgctttgctcggcagccagctggtcagccacagccacagccagcgcaggcgcagtttcgcaatcctaccaatttcgcataatgtatc

SEQ ID NO: 15 - DNA, 6529 bp, synthetic construct
  source 1..6529: mol_type = other DNA
tgcgcataatatacagcggatcggtaatcaccaccgggctcagcgccgccagcagcgcttcatgcgcgcccgcatgcatgcccaccgccaccagcgccagcgcgccaatcacggtggtcaccgggctcaggcgatcccacagcgcgctccacgcttttttttccgccgggctcgccgcttcttcctggcgcactttcaccgccagcgccggatccgcctgcgccagttcaatcagcgccatcagatgggtatcggtaattttgccgcctttgcgccacacgctcacgctgttgcggctcacgcccaggctcagcgccacgctgttatcgctatcgcggctgcacgctttgcgcgcggtatccagcagtttgttaatggtatccacgtcatattccgtttgatattagcgtgctgagccatgtgacctgcaccagctgcccgaccccgtatgataccgcgccgccggcgccgctgcgcggcccgcgccagggcgcgggcgatcgcggctttatggataccaacgcgctggcgctgattggcgcgagcgcgctgaccgtgattgtgggcctggcgcgcctggtggcgtggattctggatcgccgcgcggaagcggcgctgcgcgcgcatcgcgaacaggtgtttattattgaaagctatgcggatctggtggcgagcagcctggcggtggattatgtggatccgtgcgtggcggcggcggaagaacgcgcggaactgatggcggatgcgaccagcggctattggtatggccggtgcgccgcatgaaagaatttgcgaaatgcattggctatgtgtgggcgctggcggcgtttattagcctgtttattattggcgcgtattatctgccggaaccgtggacctgggtgtgcggcgtgtttctgtttctgaccccggcgggcctgattgcgtattttagcggcgtgggccatgaccgatgcgccgagcgtgcagtggctgattagcgcggcgcgcgtgcatggcggcccgcgcatttatgtgctgaccattgtggtgaacggcaacgcggtggaacagagctattttgcgagccgcaccgatgcggcggatgcgcgcgatgcggcgatggaattttatcgtggcggatggcacctgcagcttttgcggcgataccaccgcgtatttttttccgggccgcctgtgcctggcgtgcaccagcaaaaacgcgcgcattcgcatgcaggaacagccgacccagagccgcgaactgagcgcgtttgatgcgagcgtgggcgtgatgcaggcggcgacccgccgcaccgaaattgcggcggaaaaaattcagaaaaacaaacgcgtggtgggcaccagcgtgcgcgaatttgatgcggcgcatccgattgcgctgaccgcggaaggccagcgcgcggcgctggcgctgggcctggtgcattataaaaccagcgatacccgcgcgagcagcaccggcaccgtgaccattgaaattgatccgctgcaggcgcgcgcgcagcgcctgcgcaaaagcgtgattaccggcgcgcgcctgcatgatcaggaagcgaaaaaaggcagctttcgcggcgcgtggtattttctgaccctgacctatcgcgatggcagcgatagcagcccgcgcgatgtgagcgaactgtttaaacgcatgcgcggccattttaaccgcctgaaaagcggccgcgcgcgctggaaccgcgaaagctttcgctatgtgtgggtgggcgaactgacccagcgctttcgcccgcattatcatgtgatgctgtgggtgccgaccggcatgtattttggcaaagtggatcagcgcggctggtggccgcatggcaccacccagattgaaaaagcgcgcaactgcgtgggctatctggcgaaatatgcgagcaaatttaccgcgctgaccgcgggcgcgtttccgaaaggctttcgcacccatggcattggcggcctggataccgaaagcaaacgcgaactgcgctggtggaaagcgccgaaagatgcgcgcgaagcgctgggcggcgaagcggatattcgcaaagtgaaaggcggctggtttgatcgcctgaccggcgaattttggccgagcccgtggaaagtgacctttatttttggccgcacctttgcgtggaaagtggtgcagctatgaaagtgcaggtgatgagcagcgcggtggcggtgcgcagctttccggcgcgcgaaggcaaaccggcgacccattttcgcgaacagaccgcggcggtgctgcgcgaaggcgattttccgctgccgtttaccattggcctggatgaagatcagccgccgtatggcgaaggcttttatattattgatccgaaaagcctgcagaacaacaaatttggcggcctggaatttggccgccgcattcgcctggtgccggatctgaccgcgcgcctgcagcagcagccggcgaaagcgggctaatccatggcgatgtgcgtagccctgcaagcggaccgcgcgctggtgcccgccggcgaaccgcctggcgagcgcggcggcgacctatagcagcgtggtggcgaacaccgcgtgcatgcgctggtgcaaaaaacgcagccaatgccgagcaaagaagatgcggtggcgtggagcaccggctgctgcggcctggtgattgtgtggtttgtgctgggccgcctggcgggcagcgtggcgggcatgtttaacgatcgctaaatcaatcaatcaatgaggagagttaaacatgggcgatattctggcgggcgtgagcggcgcggaagcggcgaccgcgatgattgcggcggcggcgattattgcgctggtgggctttaccaaatggggcgcgaaaaaagtggcgagcttttttggctaattgtggtgagggtagggcggcgcttcggtgtcgccctctctctttcagggtagggcgatgatcgttctcttgttctgtggatttatgggcgcgctgtgtgggtgggcaggcgtcaagggtttagacgcgtgattaccgtgcgcctggtggcgtgcattctggcgggcgcgttttggagctgcgcgtttagcgcgaacgcggcgatgtgccgcgatagcgcggatgcgtgcgatcagggccaggcgtttctggcggcgcgcatgctggcggatcagcgcggcgtggatctgtgcaaactggtgggcggcagcaacagcctgtataaaggcccggatgtggtggcggatagcgcgggcatttataacgcgcaggcgacctgcagcattggcggcccggcgagcgcgggcagcaccttttatagcaaaacctgcgcgcagcgcccgccgctgattggcgcgagcagcagcgatggcagcggctttagctgcgatgatggctgcttttataactttaccgtgggcgcgagcggcggcagcggcatgtatccgagcggcgcgacctgcagcgcgggcgatgcgccgccgagcaccccgggcgatgatggcggcgatggcgatggcggcagcgatggcggcggcgatggcggcagcgatggcggcggcgatggcggcagcgatggcggcggcgatggcggcagcgatggcggcggcgatggcggcagcgatggcggcggcgatggcgatggcgatggcgatggcgatggcgatggcgatggcgatggcgataccccgggcgatggcgatggcaccaccccgggcgaaggcgaaggcggcgaaggcgcgccgatgagcgaactgtataaaaaaagcggcaaaaccgtggaaagcgtgctgagcaaatttaacacccaggtgcgcggcaccccgatggtggcgggcattaccgattttatgaccgtgccgagcggcggcagctgcccggtgtttagcctgcgcgcgagcaaattttgggatgcgatgaccattaactttcattgcggcggcgattttctggcgtttctgcgcgcggcgggctgggtgattctggcgattgcggcgtatgcggcgctgcgcattgcggtgacctgaggacgacgatatgcaggcgggctggtttaacgatctgaccgcgtggctgtggcgcgcggtgaaactggtgtggcaggcggtggtggattttgtgggcgatctgtttgtgatgtggctggaacagagcctggcggcgattctgtatgtgctgaccctgctgccgatgccggattttatgaaaggccagagcattggcggcatgctgggcaacgcgggcagcaccattctgtggtttgcggatgtgtttaaaattggcccggcgctggtgatgattggcgcggcgatggtgttttatctgctgcgccgcgtgctgaccgtgggcatttggtgacatgctggtgtttaacgaaggcgtgccgcgcgcgggcaaaagctatgatgcggtgaaaaaccatattctgccggcgctgaaaaaaggccgccgcgtgtttgcgcgcctgaacggcctgcgctttgatcgcattgcgaaacatctgggcattgcggaaagcgatgtgcagagcctgctggtgctggtggataccaaaaacgtgaccaaactgtttgcgtgcacccaggatgaaagcggcaaatggtgcattccggatgaatttaaagatgcgctggtggtgattgatgaagtgcatgaattttatgtgaacgaacgcaaaccgctggcgccggcggtggaaaacttttgggcgctgctgggccagaacggcggcgatgcggtgattatgacccagtggattaaccgcctgcatagcgcggtgaaagcgcgcattgaaaaaaaaaacacctttcagaaaatgaccgcgattggcatgaaaggccgctatcgcgtgacctattttcataccaccagcccgggcaaatttgaaaaagtgggcggccagaccctgaaatatgatccggcgatttttccgctgtatgatggctatgcgccgggcgcggaaaacaccgaagtgtatgaagaaggcggcaaaaacgtgtgggcggcgatggcggtgcgcgcggcgatttttattgtggtgggcgtggtgggcatttatttttttgtgcattattttaccaaagatcgcagcgatccgaacaaaccggtggcgagcgcgagccagaccaacaaaccgacccatgtgggcgcgggcctggcgaacggcgcgccgagcgtgccgattcagccgccgccgccggatccgctggcggatctgacccaggaacagcgctatgtggcggaactggcgaacaaaggccgcattcgcctgagcgcgcgcgcgcgcgtgggcgatcaggatcgcgcgtgggtgcagtggattgatgaaagcaacaacgtgattgaagaactggatctgacccagctgcgcgcgctgggctatagcgtgagcgtggtgacctatggcgtgcgcctgagcgcgggcaaacatattatggtggcgaccgcgtggccgtggaccgcgccgattcgcgaaaaagatgcgcgcctgtataacatggcgccggatggcagcggcggcgcggcgggcgtggcgaccgtgggcagcgatggcggcggcgcggatcgcgatcgcgtgcgcggcggcgtgattgaatatggcccgcgcacccagggcacctttccggataacaaagcgtataccaccaacaccaccaccccggcgaccaccctgcagatgtaggtagaatcccgctctcaaggggagggcgtatggatattcgcgcgtttctggtggtgctgtttgtggcgagcgcgccggcgagcgcgcagcaggtgtttaaatgcgtgaacggcgcgcaggtggtgtatcagagcgcgccgtgcgcgggcgtggcggcgaaacagtgggatgcgcagccggaaccggataacccggcgctgcgccagcgcctggcgcgcaccgcggaacagctgcgcgcgcgcaacgcgagcccggtgcgcagcggcagcggcacctatgtggcggcgagcagcagcaaagatcgctatgcgtgcgaagtggcgaaagaaggccgccgcgcggcgtatgaagcggcgggcgtgcatcgcagctttgcgctgagcagctattgggataacgcggtgcaggatgcgtgcaaataacccaggggtgtaggggcatagcccctacggataacgccttagccgcggcgcgggccgcgcgggccgcgggtgcggcgaatcacggtgctgcgatccgcgctgcccgcgccatccaccacgctcaggcgcatttcgcggcggcggcgcagcacatcgcgcagataaatcacttccgccgggcgcgcatgccacacgcgttcgcgttcttccgccatcatcagcgcccattcgcgcgcaatgttgcaggtcaggctccaatagcgcatgcccaccggatcaatatcgcggccttccggggtaaaaaagcgatggccctgaaagccaaagcccgcccacgggccggtcagatccacgcgatcataggtatccagggtcattgtccagtccgcttcctgtggagggaccagcattgataggccgccagggcgcacaggagcgtcaacagcccacttcgcataatgtata